(19) United States
(12) Patent Application Publication
(10) Pub. No.: US 2026/0240761 A1
(43) Pub. Date: Aug. 20, 2026

(54) ORAL FILM UNIT DOSAGE FORM

(71) Applicant: SWIPP AB, Lund (SE)

(72) Inventors: Bengt Westrin, Kivik (SE); Christer Sjögren, Viken (SE); Nicolas Rollet, Chevigny-Saint-Sauveur (FR); Justine de La Prunarède, Nuits-Saint-Georges (FR); Anders Carlsson, Stockholm (SE)

(21) Appl. No.: 19/160,948

(22) PCT Filed: Mar. 1, 2024
(86) PCT No.: PCT/EP2024/055324

§ 371 Date: Aug. 29, 2025

(30) Foreign Application Priority Data
Mar. 1, 2023 (EP) 23159305.4

(51) Int. Cl. A61K 9/00; A61K 31/5517; A61K 47/02; A61K 47/10; A61K 47/14; A61K 47/26; A61K 47/32; A61K 47/34; A61K 47/38

(52) U.S. Cl. CPC A61K 9/006; A61K 31/5517; A61K 47/02; A61K 47/10; A61K 47/14; A61K 47/26; A61K 47/32; A61K 47/34; A61K 47/38

(57) ABSTRACT

The present invention relates to oral films containing an active pharmaceutical ingredient (API) capable of providing seizure relief and one or more film-forming polymers, the use of such films, for example for the acute treatment of ongoing seizures, and methods for preparing such films.

### TECHNICAL FIELD

The present invention relates to oral films containing an active pharmaceutical ingredient (API) capable of providing seizure relief and one or more film-forming polymers, the use of such films, for example for the acute treatment of ongoing seizures, and methods for preparing such films.

### BACKGROUND

Midazolam is a benzodiazepine drug substance. It is available as injection products for a variety of indications, but also as buccal solutions (typically up to 10 mg midazolam, per pre-determined dose) for the acute treatment of ongoing seizures, and as oral solutions or syrups (typically 2 mg/mL, with recommended dose of up to 20 mg) for moderate sedation in paediatric patients prior to diagnostic or therapeutic procedures or for pre-re-sedation before induction of anaesthesia. For those two uses of midazolam, i.e., seizure treatment and sedation, it is clearly advantageous to have products that are easy-to-use and that can improve compliance and dosing reliability.

Midazolam is commercially available as medicinal product containing midazolam hydrochloride, for example in the form of oral syrups such as VERSED® which contains 2 mg/ml of midazolam (specified as the base), or BUCCOLAM® oromucosal solution which contains 5 mg/mL midazolam (specified as the base) in pre-filled syringes of 0.5 mL, 1 mL, 1.5 mL or 2 mL, i.e., pre-determined doses of 2.5 mg, 5 mg, 7.5 mg and 10 mg, respectively. Midazolam has also been marketed in the form of its maleate salt, for example in DORMICUM® tablets containing 7.5 or 15 mg per tablet, or in EPISTATUS® oromucosal solution which contains 10 mg/mL midazolam. BUCCOLAM and EPISTATUS oromucosal solutions are examples of midazolam products that are formulated for buccal administration, and VERSED oral syrup is an example of a midazolam product formulated for oral administration.

The use of oral film as dosage form has been proposed for a large number of APIs, for example amlodipine, buprenorphine, dexamethasone, donepezil, loperamide, naloxone, nicotine, ondansetron and many others. Some examples of approved drug products for which there are human pharmacokinetic or clinical data are SUBOXONE® sublingual film (buprenorphine and naloxone) used for the treatment of addiction to opioid products; BELBUCA® buccal film (buprenorphine) used for the treatment of severe pain; and SETOFILM® orodispersible film (ondansetron) used for prophylaxis or treatment of nausea and vomiting. These products also exemplify the three main categories of oral films: sublingual films, buccal films and orodispersible films (ODF), respectively, which differ with regard to the intended site of administration and the intended predominant route of absorption into systemic circulation. It is generally considered that for a sublingual film, the predominant route of absorption can be both transmucosal (i.e., absorption from the oral cavity) or oral (i.e., absorption from the gastrointestinal tract), for a buccal film it is predominately transmucosal, and for an orodispersible film it is predominately oral. There are also oral films intended for local effects (non-systemic), as well as a large number of non-prescription products (OTC).

The main advantages with oral films are generally considered to be that they are easy to use, that they do not require water for the administration, that they are especially feasible for certain patient groups (e.g., those with difficulties swallowing tablets, or those that are unconscious when the treatment is given) and in treatment of diseases or conditions where compliance can be an issue. In addition, for oral films for which the predominant absorption route is transmucosal, the so-called first-pass effect is eliminated or reduced. Moreover, the time to achieve effective plasma levels can often be faster for an oral film than for a conventional tablet.

There are also disadvantages and challenges with oral films. One example is the limitation of the strength, i.e., the content of the API, due to the minute size of an oral film. Typically, due to that, the strength of an oral film has to be 10 mg or less, although in rare cases it can be higher. This limitation of the strength is especially challenging for films in which the API is intended to be dissolved inside the film (a state which is sometimes also called “solid solution” or “molecular dispersion”). In such films, and especially at concentrations of 15 wt % and higher, the dissolved API may be prone to precipitate inside the film.

In this context, the word precipitation and precipitate refer to either a phase separation within the film or to crystallization, both of which potentially involves the API. The precipitate may wholly or partly consist of the API and may be either amorphous or crystalline or a mix thereof. If crystalline, it can be referred to as re-crystallization because the API was originally added in crystalline form before dissolved during the manufacturing. Such crystalline precipitate may have a different polymorphic form than the originally added API.

Such precipitation may have an impact on the film's appearance and dissolution rate, and even on the human bioavailability and clinical efficacy of the product. Precipitation may thus be very unbeneficial and must usually be avoided both during manufacturing and storage. If that precipitation can be avoided, however, having the API dissolved is an attractive approach because the dissolution rate for the API from the film is then usually higher than if the same amount of API would have been present as suspended, solid particles inside the film.

Another challenge is the film manufacturing, which is a less established technology than for example tablet manufacturing. The film composition has to be such that the mechanical property of the film allows for a continuous coating process and converting process, the latter of which can be rather high speed and requires strength and plasticity of the polymer-based film. Physical stability is yet another challenge, which is partly associated with the high concentrations needed to achieve the desired strengths. For example, for films of normal size and thickness, a 10 mg strength means API concentrations inside the film of about 30 wt %. For a sparingly soluble API, intended to be dissolved in the film, such a drug load can induce precipitation during storage.

The main alternative to having the API in the dissolved state in the film is to have it in a solid state, i.e., the API particles being suspended inside the film and not having undergone any changes (e.g., dissolution or recrystallization) compared with the API added as raw material. For that alternative, drug load or precipitation is usually less of challenge, but there are other potential challenges, e.g., the dissolution rate.

Finally, it must be known beforehand by the developers of oral films if the in vivo dissolution is desired to be instantaneous, or desired to be moderately fast, or desired to be very slow. That choice of course depends on the desired in vivo absorption rate; usually (albeit not always) increased in vivo dissolution rate also gives an increased in vivo absorption rate. But the choice can also be driven by a desire to minimize such losses of the administered dose that occur if the patient is drooling (as can be the case during a seizure) or if the patient tries to spit saliva after administration (as can be the case for example in moderate sedation of children). If the film has an instantaneous in vivo dissolution, the entire dose will instantaneously be mixed with the saliva in the mouth and thus be prone to losses due to drooling or spiting. In the other extreme case, i.e., a very slow in vivo dissolution, it will take time for the main part of the dose to mix with the saliva, and the absorption into systemic circulation will be slower and potentially also lower. Hence, a film with moderately high dissolution rate is preferred in many of the medical applications for oral films, especially if the patient is unconscious or conscious but non-collaborative, in order to avoid dose losses while at the same time not resulting in a too slow in vivo absorption.

Likewise, in case the oral film is intended for buccal or sublingual administration and contains an API for which the buccal-transmucosal bioavailability is higher than the oral-gastrointestinal bioavailability, the inevitable and normal swallowing of saliva, which will contain some of the administered API, will induce a de facto decrease in the overall bioavailability compared with the hypothetical situation that there would be no swallowing of saliva. Thus, with the same rationales as presented above for drooling or spitting, this speaks in favour for a film with moderately high in vivo dissolution rate, because the saliva (some of which is inevitably being swallowed) will then contain less of the administered dose and there will be less de facto decrease in the overall bioavailability.

Likewise, in case the oral film—regardless of being formally categorized as sublingual, buccal or orodispersible—is placed on the tongue (i.e., oral administration) but contains an API for which the buccal-transmucosal bioavailability is higher than the oral-gastrointestinal bioavailability, it will again be beneficial if the dissolution is not instantaneous. Because, if instantaneous dissolution occurs when the film is on the tongue, almost the entire dose will go the oral-gastrointestinal route instead of first getting a possibility to be absorbed along the buccal-transmucosal absorption route which—in this hypothesized case—is superior to the oral-gastrointestinal route.

The development of oral films containing midazolam is a case when all of the various considerations mentioned above carry significant relevance. Midazolam (base) is a sparingly soluble API, and its buccal-transmucosal bioavailability is higher than the oral-gastrointestinal bioavailability. Furthermore, it is being widely used in the treatment of ongoing, acute seizures and for inducing moderate sedation, respectively, which are situations where drooling, swallowing and/or spitting out saliva may be an issue. Jithendra et al. 2015 describes a film prepared by first preparing a solid dispersion consisting of midazolam and other excipients (e.g., PEG-4000, poloxamer-188, and hydroxypropyl β-cyclodextrin), then “pulverizing” that material and finally using this pulverized material as a carrier for the active ingredient for solvent casting preparation of a buccal film with hydroxypropyl methylcellulose (HPMC) as film-forming polymer. The rationale for this two-staged approach is to improve the solubility/dissolution rate of the API, and “very rapid release” is eventually achieved. The midazolam concentration in the films is not explicitly reported by Jithendra et al. 2015 but it can be deduced that the concentration would vary between approximately 2.3 wt % and approximately 8.3 wt %, for a film comprising 10 mg midazolam.

Soroushnai et al. 2018 describes another two-staged approach, with the rationale to incorporate a “high drug dose” despite “midazolam's high lipophilicity and poor water solubility”. Midazolam hydrochloride is used, with which a midazolam nanosuspension is first prepared, by a high-pressure homogenization technique, using N-trimethyl chitosan, Tween-80 and polaxamer-188 as excipients. Next, the nanosuspension is freeze-dried, and finally this freeze-dried material is used for solvent casting preparation of a “fast-dissolving oral film” with hydroxypropyl methylcellulose or pullulan as film-forming polymer. The reported midazolam concentration in the film is 15 wt %.

WO 2017/009446 describes a “bio-adhesive film or wafer” which is prepared in a more conventional way, i.e., solvent casting preparation without any preceding preparation of a midazolam intermediate material. HPMC is used as a film-forming polymer. The intended films are described as having 0.25-2 mg midazolam strengths or even as low as 0.1 mg, or are described as typically containing 0.5-20 mg midazolam per gram of film, which corresponds to 0.05-2 wt % of midazolam in the film.

Rogawski et al 2019 discloses a buccal film containing diazepam but does not disclose any information about the film design or composition, other than that HPMC is used as a film-forming polymer.

CN1830447A describes a film containing midazolam maleate, and for which the film-forming polymer is either PVA or HPMC, and the plasticizer is either PEG-400 or glycerol. It is described that the dissolution rate is 7 times higher than a tablet and that all components dissolve within 30 seconds.

U.S. Pat. No. 10,744,086 B2 describes the preparation of freeze-dried, porous wafers containing midazolam, intended for administration to the oral cavity. Amyloptectin is the main polymeric component, and the concentration of midazolam, after completed freeze-drying, is 3.7 wt %. “BP basket” and USP paddle methods are used to study the dissolution rate, showing complete or almost complete dissolution after 0.5 minutes with both methods. Technically speaking, a “porous wafer” is not the same thing as an oral film as herein described, but usually it has similar dimensions and similar purpose as an oral film.

U.S. Pat. No. 11,173,114 B1 describes the preparation of oral films by firstly depositing a liquid composition containing a film-forming polymer into a small well, and then depositing a liquid composition containing the API on top of that, and then subject the resulting mix to drying, to obtain a film. No example of any specific API is given. Among possible solvents to be used when preparing the liquid compositions, ethyl acetate and 2-propanol are mentioned, though not being used in any if the experimental examples presented.

US 2017/0119660 A1 describes the preparation of a sublingual or buccal dosage form, by firstly preparing a clear, homogeneous aqueous solution containing two or more “complementary” film-forming polymers, and then adding a solution consisting of an amphiphilic API (being a base or a salt) dissolved in one or more organic solvents and then drying the resulting solution, which is clear and homogeneous, and eventually preparing the dosage form which may be for example a film, a tablet, a disk or a powder. Said solvent is selected so that it does dissolve the API, is miscible with water, and does not cause precipitation of the polymers when the second solution (containing the active) is added to the first solution. As one of several possible APIs in this invention, midazolam is mentioned, though not being used in any if the experimental examples presented. As possible organic solvents to be used when preparing the second solution, 2-propanol, 1-pentanol and others are mentioned.

There is thus a need for the development of an oral film containing 10 mg or more of midazolam or a pharmaceutically acceptable salt thereof, yet having an area and a thickness that are feasible for oral films which means that the concentration of midazolam inside the film can become very high, e.g., 30 wt %. Despite such high concentration, the film must be chemically and physically stable, e.g., the midazolam must not precipitate during manufacturing or during storage. Furthermore, the film should have moderately high dissolution rate, to prevent a partial loss of the administered dose in case the patient is drooling, spitting or swallowing saliva after the administration of the film.

### SUMMARY

The present inventors have developed a unit dosage form in the form of an oral film with a high concentration of midazolam or a pharmaceutically acceptable salt thereof. In said oral film, the API is suspended inside the film, i.e., not dissolved, and the film has a moderately high dissolution rate. The present inventors have also demonstrated that a midazolam oral film with a moderately high dissolution rate has a much higher bioavailability—after buccal administration—than a buccal solution of the same strength. It has also been demonstrated to have much higher bioavailability—after oral administration—than an oral solution of the same strength.

In one aspect, the present invention relates to a unit dosage form in the form of an oral film comprising:
a) at least 20 wt % (defined as the base) midazolam or a pharmaceutically acceptable salt thereof; and
b) 35 to 80 wt % of a film-forming polymer which is soluble in ethyl acetate.

In one aspect, the present invention relates to a unit dosage form in the form of an oral film comprising:
a) at least 20 wt % (defined as the base) midazolam or a pharmaceutically acceptable salt thereof; and
b) 35 to 80 wt % of a film-forming polymer selected from the group consisting of:
i. HPMC 2528;
ii. HPC;
iii. hypromellose acetate succinate;
iv. methacrylic acid-methyl acrylate copolymers; and
v. polyvinyl caprolactam-polyvinyl acetate-polyethylene glycol graft copolymer.

In one aspect, the present invention relates to a process for producing a unit dosage as defined herein, the process comprising the steps of:
a) Mixing the API and one or more film-forming polymers in one or more solvents to provide a wet mix.
b) Casting the wet mix obtained in step a) to provide a wet film.
c) Drying said wet film to obtain the final, dry film.
d) Converting said film into unit dosages by cutting it into feasible sizes and packaging these into primary containers.

In one aspect, the selection of film-forming polymer(s) and solvent(s) in that process is such that the film-forming polymer(s) but not the API dissolves in the wet mix in step a), thereby achieving a final film in step c) in which the API is suspended as solid particles inside the film instead of being dissolved.

In one aspect, the present invention relates to a unit dosage form as defined herein for use in the acute treatment of seizures in a human subject.

In one aspect, the present invention relates to a unit dosage form as defined herein for use in the induction of moderate sedation or pre-sedation in a human subject.

### DESCRIPTION OF DRAWINGS

FIG. 1. Appearance of formulation A7. Left (A): Visual appearance of formulation A7 after storage. Right (B): Microscopy of same sample, with about 5-10 times magnification compared with the left picture.

FIG. 2. XRPD diffractogram for formulation A19. Bottom: Reference diffractogram recorded for the API midazolam base (fresh sample). Middle: Formulation A19 at start of XRPD study. Top: Formulation A19 after 4+4 weeks storage exposed to ambient temperature and 65% relative humidity (RH), followed by 4 weeks at 40° C. and 75% RH, followed by 2 weeks at 40° C. and 89% RH. (The vertical shifts between the diffractograms are made for clarity and do not represent actual differences in intensity. The diffractograms were not recorded at the same occasion).

FIG. 3. In vitro dissolution results for formulation A18. The two lower, coinciding curves are for formulation A18 with dissolution media being phosphate buffer pH6.8+Tween 20, and water, respectively. The upper curve is included as a reference and shows formulation A24 (in phosphate buffer pH6.8+Tween 20) which was the clinical batch used in Example 23, also referred to as CB2 (“clinical batch 2”). All curves have been normalized, i.e., 100% represents the terminally recorded value.

FIG. 4. Solvent screening setup. A: Ethanol as solvent being screened. B: Ethyl acetate as solvent being screened. The pictures show the actual results for these two solvent but the aim of FIG. 4 is just to demonstrate the experimental setup and how results were recorded.

FIG. 5. XRPD diffractogram for sedimented material from solvent screening. Bottom: Reference diffractogram recorded for the API midazolam HCl. Middle: Sediment formed in solvent screening with ethyl acetate. Note that this diffractogram is no different from the reference. Top: Sediment formed in solvent screening with ethanol, which is different from the reference. (The vertical shifts between the diffractograms are made for clarity and do not represent actual differences in intensity. The diffractograms were not recorded at the same occasion).

FIG. 6. XRPD diffractogram for formulation A22. Bottom: Reference diffractogram recorded for the API midazolam hydrochloride (fresh sample). Middle: Formulation A22 at start of XRPD study. Top: Formulation A22 after 4+4 weeks storage exposed to ambient temperature and 65% relative humidity (RH), followed by 4 weeks at 40° C. and 75% RH. (The vertical shifts between the diffractograms are made for clarity and do not represent actual differences in intensity. The diffractograms were not recorded at the same occasion).

FIG. 7. In vitro dissolution result for formulation A22. In vitro dissolution results for formulation A22, supplemented with reference curves for formulation A24 which was the clinical batch used in Example 24, also referred to as CB2 (“clinical batch 2”), and for the API midazolam hydrochloride batch which was used when preparing A22 and A24. All curves have been normalized, i.e., 100% represents the terminally recorded value. Studies were carried out to 120 minutes but here only up to 15 minutes is shown.

FIG. 8. Plasma curves from clinical study with buccal administration of formulation A23 (CB1). The curves represent mean values of the 24 study subjects' plasma concentrations of midazolam (in ng/ml), versus time. Triangles represent a 10 mg dose of the midazolam oral film formulation A23, and circles represent a 10 mg dose of Buccolam oromucosal solution, both of which were buccally administered.

FIG. 9. Plasma curves from clinical study with oral administration of formulation A24 (CB2). The curves represent mean values of the 22 completing study subjects' plasma concentrations of midazolam (in ng/ml), versus time. T2 (circles) represents a 10 mg dose of the midazolam oral film formulation A24, and R1 (triangles) represents a 10 mg dose of Midazolam Hydrochloride Syrup 2 mg/mL, both of which were orally administered.

FIG. 10. Box plots from clinical study with oral administration of formulation A24 (CB2). The box plots are aimed to demonstrate the variabilities for the products with regard to Cmax (ng/mL) and how they compare with each other. T2 (right) represents a 10 mg dose of the midazolam oral film, and R1 (left) represents a 10 mg dose of Midazolam Hydrochloride Syrup 2 mg/mL, both of which were orally administered. The bottoms and tops of the boxes represent the first and third quartile, respectively. The whiskers outside the boxes represent the lowest and highest value, respectively. The solid lines inside the boxes represent mean values and the dashed lines the median values.

FIG. 11. XRPD diffractogram for formulation with HPC. Bottom: Reference diffractogram recorded for the API midazolam hydrochloride (fresh sample). Top: Film formulated with HPC from Example 25. (The vertical shifts between the diffractograms are made for clarity and do not represent actual differences in intensity. The diffractograms were not recorded at the same occasion).

### DETAILED DESCRIPTION

### Unit Dosage Form

In one aspect, the present invention relates to a unit dosage form in the form of an oral film comprising an active pharmaceutical ingredient (API) and a film-forming polymer. The terms “active pharmaceutical ingredient”, “API” and “drug substance” are used herein with the same meaning. If it specifically refers to the raw material actually being added during manufacturing, that is specified or is obvious from the context. The term “unit dosage form” refers to physically discrete units suitable as unitary dosage for subjects undergoing treatment, with each unit containing a predetermined quantity (dose) of API calculated to produce the desired therapeutic effect. The term “oral film” as used herein collectively refers to sublingual, buccal and orodispersible films (ODF), as well as any other films placed into the oral cavity aiming at systemic or local effects.

In one embodiment, the unit dosage form is for buccal administration. The term “buccal administration” as used herein refers to administration to the space in the oral cavity that is outside the teeth (when the jaws are closed) such as for example the inside of the cheek or under the upper lips. An oral film intended for buccal administration is usually referred to as a “buccal film”.

The word “oromucosal” is sometimes used synonymously with “buccal”, for example in terms like “oromucosal administration” or “oromucosal solutions”, but the word “oromucosal films” is less frequently used.

Following buccal administration of an API by an oral film or other buccal dosage form, the desired, predominant absorption route into systemic circulation is typically transmucosal which may also be called buccal, or buccal-transmucosal to avoid misunderstandings.

In one embodiment, the unit dosage form is for sublingual administration. An oral film intended for sublingual administration is usually referred to as a “sublingual film”.

Following sublingual administration of an API by an oral film or other sublingual dosage form, the desired, predominant absorption route into systemic circulation is either transmucosal or oral.

In one embodiment, the unit dosage form is for oral administration, i.e., applied on a site that is inside the teeth (when the jaws are closed), yet not being sublingual. For example, onto the tongue. An oral film intended for such oral administration is usually referred to as an “orodispersible film” or “ODF”.

Following such oral administration of an API by an oral film or other oral dosage form, the desired, predominant absorption route into systemic circulation is oral (i.e., oral-gastrointestinal).

In one embodiment, the film is a mucoadhesive film.

In one embodiment, the film has a moderately high dissolution rate. The meaning of “moderately high dissolution rate” is explained below.

To avoid a very slow in vivo dissolution rate, especially if the API as such has a very low solubility or dissolution rate, it may be favourable if the API is dissolved inside the film, i.e., not suspended. For the same reason, the film should not be too thick, though yet thick enough to accommodate the intended dose of the API. Furthermore, the unit dosage form should be stable for storage. Such storage stability does not just comprise chemical stability, but also physical stability for example that the API, if intended to be dissolved in the film, should stay dissolved and not precipitate during storage.

If the other approach is chosen, i.e., that the API is suspended as solid particles inside the film i.e., not dissolved, it is especially important to find ways to avoid a very slow in vivo dissolution rate because solid particles inside the film can be expected to result in a slower dissolution compared with the case that the same amount of API is dissolved in a film of the same composition.

Thus, in one embodiment, the API is present in the form of solid, suspended particles inside the film.

### Dissolution Rate

The terms “dissolution” and “dissolution rate” used herein have the same meaning as, for example, for a tablet, i.e., the rate with which the API becomes available in an aqueous solution surrounding the dosage form. The terms dissolution and dissolution rate apply both to the in vitro situation, for which there are several, standardized and widely accepted assessment methods, and to the in vivo situation, for which the methods are not as standardized or widely accepted. In this application, it is usually specified in the text whether “dissolution” and “dissolution rate” refer to the in vitro or the in vivo situation. If not specified, it is either evident from the context or it refers to both situations.

However, as oral films are designed to dissolve in the mouth (albeit with different desired rates), the terms “dissolution” and “dissolution rate” may also refer to the film as such, rather than to the API. Typically, whether subject to in vivo or in vitro dissolution, an oral film typically starts to dissolve rather quickly after coming into contact with the aqueous surroundings, then continues to dissolve and maybe also starting to disintegrate into pieces, and finally becoming completely dissolved.

It should thus be understood that dissolution and dissolution rate of the API in an oral film is not conceptually the same as the dissolution and dissolution rate of the film as such. These two dissolution processes are different on the molecular level, and the dissolution rate of the API can be both faster and slower than the dissolution rate of the film. If not otherwise specified in this document, the terms “dissolution” and “dissolution rate” in the present application means those of an API.

Typically, in vitro and in vivo dissolution rates correlates, at least semi-quantitatively.

The in vitro dissolution rate may be measured using the United States Pharmacopeia (USP) Dissolution Apparatus 2—Paddle, with sinkers, 1000 mL phosphate buffer with pH 6.8, or other dissolution medium if specified, at 37° C.±0.5° C.), and stirring speed 75 rpm. See for example Example 8 for further details. That method is referred to as “USP Dissolution Apparatus 2” in the various embodiments below, as well as in Items. Other dissolution media may be phosphate buffers with other pH and/or with the addition of solubilizers such as Tween 20, or other buffer types, or just water.

In a preferred embodiment, the unit dosage form of the present invention has a moderately high in vitro dissolution rate. The term “moderately high in vitro dissolution rate” as used herein means that at least 85% of the API has been dissolved within 10 minutes in the USP Dissolution Apparatus 2—Paddle, but no more than 95% has been dissolved within 5 minutes. Hence, the term “moderately high dissolution rate” does not include instantaneous dissolution, such as for example the film being completely dissolved and/or all API released in vitro within one minute. It is an aim of the present invention to provide a unit dosage form which does not have an instantaneous in vivo dissolution and wherein the administered dose is thus not instantaneously mixed with saliva, since such film may suffer from losses of the administered dose due to drooling, spitting or swallowing, as explained in the Background section above.

In one embodiment, at least 85% of the API has been dissolved within 10 minutes in the USP Dissolution Apparatus 2—Paddle, such as at least 90%, such as at least 95% of the API has been dissolved within 10 minutes.

In one embodiment, the API is midazolam, or a pharmaceutically acceptable salt thereof, and at least 85% of the midazolam has been dissolved within 10 minutes in the USP Dissolution Apparatus 2—Paddle, such as at least 90%, such as at least 95% of the midazolam has been dissolved within 10 minutes.

In one embodiment, wherein at least 90% of the midazolam has been dissolved within 10 minutes in the USP Dissolution Apparatus 2—Paddle, such as at least 95%, such as at least 98%, such as 100% of the midazolam, or pharmaceutically acceptable salt thereof, has been dissolved within 10 minutes.

In one embodiment, at least 85% of the midazolam, or pharmaceutically acceptable salt thereof, has been dissolved within 5 minutes in the USP Dissolution Apparatus 2—Paddle, such as at least 90%, such as at least 95% of the midazolam, or pharmaceutically acceptable salt thereof, has been dissolved within 5 minutes.

In one embodiment, at least 90% of the midazolam has been dissolved within 5 minutes in the USP Dissolution Apparatus 2—Paddle, such as at least 95%, such as at least 98%, such as 100% of the midazolam, or pharmaceutically acceptable salt thereof, has been dissolved within 5 minutes.

In one embodiment, no more than 90% of the midazolam, or pharmaceutically acceptable salt thereof, has been dissolved within 1 minute in the USP Dissolution Apparatus 2—Paddle.

In one embodiment, no more than 85% of the midazolam, or pharmaceutically acceptable salt thereof, has been dissolved within 1 minute in the USP Dissolution Apparatus 2—Paddle, such as no more than 80%, such as no more than 75%, such as no more than 70%, such as no more than 65%, such as no more than 60%, such as no more than 55%, such as no more than 50%, such as no more than 45%, such as no more than 40% of the midazolam, or pharmaceutically acceptable salt thereof, has been dissolved within 1 minute.

In one embodiment, no more than 95% of the midazolam, or pharmaceutically acceptable salt thereof, has been dissolved within 5 minutes in the USP Dissolution Apparatus 2—Paddle.

In one embodiment, no more than 95% of the midazolam, or pharmaceutically acceptable salt thereof, has been dissolved within 5 minutes in the USP Dissolution Apparatus 2—Paddle, such as no more than 90%, such as no more than 85%, such as no more than 80%, such as no more than 75%, such as no more than 70%, such as no more than 65%, such as no more than 60%, such as no more than 55%, such as no more than 50%, such as no more than 45%, such as no more than 40% of the midazolam, or pharmaceutically acceptable salt thereof, has been dissolved within 5 minutes.

In one embodiment, at least 85% of the midazolam, or pharmaceutically acceptable salt thereof, has been dissolved within 10 minutes but no more than 95% has been dissolved within 5 minutes in the USP Dissolution Apparatus 2—Paddle.

In one embodiment, at least 90% of the midazolam, or pharmaceutically acceptable salt thereof, has been dissolved within 10 minutes but no more than 90% has been dissolved within 5 minutes in the USP Dissolution Apparatus 2—Paddle.

In one embodiment, at least 5% but not more than 55% of the API has been dissolved within 5 minutes, and at least 30% but no more than 70% has been dissolved within 10 minutes, and less than 90% has been dissolved within 20 minutes, when USP Dissolution Apparatus 2—Paddle is conducted at 37° C.±0.5° C., 75 rpm in 1000 mL phosphate buffer pH 6.8 with or without 0.08% Tween20.

### Active Pharmaceutical Agent (API)

In one embodiment, the unit dosage form of the present invention comprises an API that is capable of providing relief from ongoing seizures. In one embodiment, the API is an anticonvulsant. In one embodiment, the API has a sedative effect, such as an API being useful in moderate sedation before diagnostic, therapeutic or surgical procedures or pre-sedation before anaesthesia. In one embodiment, the API is selected from the group consisting of benzodiazepines and benzodiazepine-like substances.

The term “benzodiazepine” as used herein refers generically to a class of drugs substances that act as central nervous system depressants with sedative, hypnotic, anxiolytic, anticonvulsant, muscle relaxant, and amnesic actions through the positive modulation of the GABA-A receptor complex.

The term “benzodiazepine-like substances” (also known as nonbenzodiazepines or Z-drugs) refers to a class of compounds which pharmacodynamics are almost identical to benzodiazepines and therefore exhibit similar benefits, side-effects, and risks. However, benzodiazepine-like substances differ from benzodiazepines on a molecular level.

In one embodiment, the API is selected from the group consisting of midazolam, diazepam, alprazolam, brotizolam, cinolazepam, clizolam, clobazam, clonazepam, clonazolam, clorazepate, cloxazolam, diclazepam, estazolam, flubromezepine, flunitrazepam, flurazepam, flutoprazepam, kvazepam, lorazepam, loprazolam, lormetazolam, metizolam, nitrazepam, oxazepam, phenazepam, temazolam, triazolam and pharmaceutically acceptable salts thereof. In one embodiment, the API is midazolam, diazepam, clobazam, clonazepam, lorazepam or a pharmaceutically acceptable salt thereof. In one embodiment, the API is midazolam, diazepam, or a pharmaceutically acceptable salt thereof.

In a preferred embodiment, the API is midazolam (8-chloro-6-(2-fluorophenyl)-1-methyl-4H-imidazo[1,5-a][1,4]benzodiazepine, CAS number 59467-70-8) or a pharmaceutically acceptable salt thereof. Said pharmaceutically acceptable salt may be selected from hydrochloride or maleate. In one embodiment, the API is midazolam hydrochloride.

In one embodiment, the unit dosage form comprises at least 2.5 mg of API, such as at least 5 mg, such as at least 10 mg of API. In one embodiment, the unit dosage form comprises no more than 20 mg of API, such as no more than 15 mg, such as no more than 10 mg of API. In one embodiment, the unit dosage form comprises 2.5 to 20 mg of API, such as 5 to 15 mg, such about 10 mg, such as about 7.5 mg, such as about 5 mg of API. In one embodiment, the unit dosage form comprises about 10 mg midazolam. In one embodiment, the unit dosage form comprises about 7.5 mg midazolam. In one embodiment, the unit dosage form comprises about 5 mg midazolam.

In one embodiment, the concentration of API in the film is at least 10 wt %, such as at least 15 wt %, such as at least 20 wt %, such as at least 25 wt %, such as at least 30 wt %, such as at least 40 wt %. In one embodiment, the concentration of API in the film is no more than 80 wt %, such as no more than 70 wt %, such as no more than 60 wt %, such as no more than 50 wt %, such as no more than 40 wt %. In one embodiment, the concentration of API in the film is in the range of 10 to 60 wt %, such as in the range of 20 to 50 wt %, such as in the range of 30 to 40 wt %.

In one embodiment, the API is midazolam, and to achieve a strength of 10 mg midazolam of the unit dosage form, yet maintaining a feasible film thickness allowing for a moderately high film dissolution rate (such as for example 70 to 110 μm thickness) and a convenient size (e.g., 1.5×2.5 cm), the concentration of midazolam in the dry film must be about 25 to 40 wt %.

The term “strength” is used herein to describe the content of the active pharmaceutical ingredient in the unit dosage form and is typically expressed in milligram (mg) or microgram (μg). As an oral film is a unit dose dosage form, said strength is typically identical to the dose to be administered to the patient, although sometimes more than one unit dosage can be administered and sometimes just a part of one unit dosage is administered. For a film with dimensions of 15×15 mm, and a coat weight of 100 g/m², a strength of 10 mg means that the concentration of API is about 27 wt % inside the film. The term “coat weight” will be explained below.

In one preferred embodiment, the midazolam or the acceptable pharmaceutical salt thereof is midazolam hydrochloride.

When the API is a salt of a base, the information about dose or strength usually refers to the amount of the free base. For example, a dose or strength of “10 mg midazolam” refers to 10 mg of midazolam of the free base, even if said midazolam was added as a salt during the preparation of the unit dosage form and even if it remains as the salt form in the unit dosage form after completed preparation. However, sometimes when compositions or formulations (rather than doses or strengths) are described in this patent application, quantities (e.g., mg, kg, wt % or %) may refer to the midazolam salt and not to the base. This will be specified or being evident from the context.

To avoid a very slow dissolution rate, it may be favourable if the API is dissolved in the film.

However, to improve the chemical and physical stability, it may sometimes be more favourable if the API is suspended in the film, i.e., not dissolved, and that the suspended API particles have not undergone any changes in size or polymorphic form compared with the API raw material used for the preparation.

In one embodiment, the API exists predominately in such suspended and unchanged state inside the film. In one embodiment, at least 90 wt %, such as at least 95 wt %, such as at least 98 wt % of the API exists in such suspended, unchanged state in the film. In one embodiment, the concentration of API in the film is in the range of 15 to 35 wt % and at least 60 wt %, such as at least 70 wt %, such as at least 80 wt %, such as at least 90 wt %, such as at least 95 wt %, such as at least 98 wt % of the API exists in such suspended, unchanged state in the film.

If such suspended API particles inside the film have a very low dissolution rate, for example due to low aqueous solubility of the API, or due to large particles size and/or low intrinsic API dissolution rate, the in vivo dissolution rate may become very low and that may result in a slow or low in vivo absorption. Therefore, when using the approach with suspended API inside an oral film, the thickness and the composition of the film must be such that a very slow in vivo dissolution rate is avoided, unless specifically desired.

In one embodiment, the oral film contains midazolam or a pharmaceutically acceptable salt thereof and has a moderately high dissolution rate.

In one embodiment, the unit dosage form comprises two or more active pharmaceutical ingredients. In one embodiment, the total concentration or amount or API in a unit dosage form comprising two or more active pharmaceutical ingredients is equal to any of the levels presented above.

### Film-Forming Polymers

The unit dosage form of the present invention comprises one or more film-forming polymers.

In one embodiment, the film-forming polymer is selected from the group consisting of acrylates, alginates, carrageenan, cellulose derivatives, chitosan, collagen, dendritic polymers, gelatin, gum, hyaluronic acid, maltodextrin, pectin, polyethylene glycol, polyethylene oxide, polylactic acid and derivatives or copolymers thereof, polysaccharides, pullulan, polyvinylpyrrolidone, scleroglucan, starch, starch derivatives, and polyvinyl alcohol.

In one embodiment, the film-forming polymer is selected from the group consisting of: HPMC 2528; HPMC 1828; hypromellose acetate succinate; methacrylic acid-methyl acrylate copolymers; and polyvinyl caprolactam-polyvinyl acetate-polyethylene glycol graft copolymer.

In one embodiment, the film-forming polymer is selected from the group consisting of: HPMC 2528; HPC; hypromellose acetate succinate; methacrylic acid-methyl acrylate copolymers; and polyvinyl caprolactam-polyvinyl acetate-polyethylene glycol graft copolymer.

In one embodiment, the film-forming polymer is selected from the group consisting of: HPMC 2528; HPC; methacrylic acid-methyl acrylate copolymers; and polyvinyl caprolactam-polyvinyl acetate-polyethylene glycol graft copolymer.

In one embodiment, the film-forming polymer is HPMC (hypromellose) which is a cellulose derivative. The term “HPMC” as used herein refers to hydroxypropyl methylcellulose, with CAS number 9004-65-3 and E number E464. HPMC is a partly O-methylated and O-(2-hydroxypropylated) cellulose and is available in several grades that differ in the substitution type as well as in molecular weight and viscosity. HPMC types may thus be given a four digit number describing the substitution type. The first two digits refer to the percentage (w/w) of methoxy-groups, while the second two digits refer to the percentage of hydroxypropoxy-groups.

The structure of the HPMC, the substitution type, and the molecular weight give rise to viscoelastic properties. As a supplement to being categorized by the substitution type, the different HPMC grades can therefore also be categorized based on their apparent viscosity. The common way to describe that is by the apparent viscosity (mPas) of a 2 wt % aqueous solution. In one embodiment, the film-forming polymer is a HPMC grade with a viscosity of at least 1 mPas, such as about 3 mPas, such as about 4 to 5 mPas, such as about 5 mPas, such as at least 10 mPas, such as about 15 mPas, such as about 50 mPas. In one embodiment, the HPMC grade has a viscosity of no more than 100 000 mPas, such as no more than 15 000 mPas, such as no more than 5 000 mPas, such as no more than 1 000 mPas, such as no more than 500 mPas, such as no more than 100 mPas.

As used herein, “HPMC Pharmacoat 603” refers to Hypromellose 2910, 3 mPas. As used herein, “HPMC Metolose 60SH-50” refers to Hypromellose 2910, 50 mPas, It is however understood that the numbers describing the substitution type are not exact, but represents a typical interval. For example, substitution type 2910 may comprise for example 28-30% methoxy content and 7-12% hydroxypropoxy content.

In one embodiment, the film-forming polymer is an HPMC with a substitution type of about 22-28% for methoxy and about 25-31% for hydroxypropoxy, such as 23-27% or 24-26% for methoxy and such 26-30% or 27-29% for hydroxypropoxy. In one embodiment, the film-forming polymer is HPMC 2528. In one embodiment, that HPMC has a viscosity of 50-300 mPas, such as 100-200 mPas or 130-170 mPas. In one embodiment, the film-forming polymer is HPMC 2528 with a viscosity of 150 mPas. In one embodiment, the film-forming polymer is HPMC with the trade name Affinisol HPMC HME 15 LV. In one embodiment, the film-forming polymer is HPMC with the trade name Affinisol HPMC HME 100LV.

In one embodiment, the film-forming polymer is HPMC 1828 which has a substitution type of about 16-20% for methoxy and about 26-30% for hydroxypropoxy, such as about 18% for methoxy and about 28% for hydroxypropoxy. In another embodiment, the film-forming polymer is not HPMC 1828. In one embodiment, the film forming-polymer is not a HPMC has a substitution type of about 16 to 20% for methoxy and about 26 to 30% for hydroxypropoxy, such as not about 18% for methoxy and not about 28% for hydroxypropoxy.

In one embodiment, the film-forming polymer is an HPMC with a substitution type of about 22-28% for methoxy, such as 23-27% or 24-26% for methoxy, such as 25% for methoxy.

In one embodiment, the film-forming polymer is HPC which is a cellulose derivative. The term “HPC” as used herein refers to hydroxypropyl cellulose, with CAS number 9004-64-2 and E number E463. HPC is available in several grades that differ in the substitution type as well as in molecular weight and viscosity.

In one embodiment, the film-forming polymer is HPC with the trade name Klucel. In one embodiment, the film-forming polymer is HPC with the trade name Klucel EF. In one embodiment, the film-forming polymer is HPC with the trade name Klucel ELF.

In one embodiment, the film-forming polymer is selected from acrylates, acrylic polymers and co-polymers thereof; polyacrylic acids, polymethacrylates and co-polymers thereof, and polyvinyl alcohol-polyethylene glycol graft-copolymers (for example Kollicoat, such as Kollicoat IR, which is a polymer consisting essentially of 75% polyvinyl alcohol units and 25% polyethylene glycol units).

In one embodiment, the film-forming polymer is a methacrylic acid-methyl acrylate copolymer. In one embodiment, the film-forming polymer is selected from a range of a methacrylic acid-methyl acrylate copolymers with the trade name Eudragit. In one embodiment, the film-forming polymer is selected from Eudragit E 100, Eudragit RS 100, Eudragit RL 100 and Eudragit RL PO.

In one embodiment, the film-forming polymer is a polyvinyl caprolactam-polyvinyl acetate-polyethylene glycol graft copolymer. In one embodiment, that polyvinyl caprolactam-polyvinyl acetate-polyethylene glycol graft copolymer is a product with the trade name Soluplus.

In one embodiment, the film-forming polymer is a hypromellose acetate succinate. In one embodiment, that a hypromellose acetate succinate is a product with the trade name AQOAT AS-LG. In another embodiment, the film-forming polymer is not a hypromellose acetate succinate.

In one embodiment, the film-forming polymer is gum selected from the group consisting of acacia gum, guar gum, tragacanth gum, xanthan gum and diutan gum.

In one embodiment, the film-forming polymer is alginate selected from the group consisting of sodium alginate, potassium alginate, ammonium alginate, calcium alginate, propylene glycol alginate, alginic acid and mixtures thereof. In one embodiment, the alginate is sodium alginate, potassium alginate or ammonium alginate, or a mixture thereof. In one embodiment, one or more of these alginate salts comprises from 25 to 35 wt % by weight of α-D-mannuronate and/or from 65 to 75 wt % by weight of α-L-guluronate, and a mean molecular weight of from 30,000 g/mol to 90,000 g/mol.

In one embodiment, the unit dosage form comprises at least 35 wt % film-forming polymer, such as at least 45 wt %, such as at least 50 wt %, such as at least 55 wt %, such as at least 60 wt %, such as at least 65 wt % film-forming polymer.

In one embodiment, the unit dosage form comprises no more than 80 wt % film-forming polymer, such as no more than 70 wt % such as no more than 65 wt %, such as no more than 60 wt %, such as no more than 55 wt %, such as no more than 50 wt %, such as no more than 45 wt % film-forming polymer.

In one embodiment, the unit dosage form comprises 35 to 70 wt % film-forming polymer, such as 45 to 70 wt %, such as 50 to 60 wt %, such as 55 to 65 wt % film-forming polymer. In one embodiment, the unit dosage form comprises 35 to 70 wt % HPMC, such as 45 to 70 wt %, such as 50 to 65 wt %, such as 55 to 60 wt % HPMC.

In one embodiment, the film-forming polymer has been identified and selected by a screening procedure with the following elements:

1) A number of non-toxic, volatile solvents are screened for their ability to dissolve the API.

2) Those solvents that are not dissolving the API are selected. The criterium used for “not dissolving the API” is: in an API:solvent mix with 5:95 weight ratio, not more than 5 wt % of the added API should be dissolved after stirring for 2 hours at room temperature.

3) A number of film-forming polymers are then screened for their ability to be dissolved in the solvent(s) selected in step 2. The criterium used for “being dissolved in the solvent(s)” is: in an polymer:solvent mix with 15:85 ratio, not more than 5 wt % of the added polymer should remain non-dissolved after stirring for 2 hours at room temperature.

4) Finally, selecting the one or more polymer that are thus being dissolved in step 3) by the solvent(s) selected in step 2).

### Plasticizer

The mechanical properties of the film must allow for a continuous coating process and converting process, the latter of which can be rather high-speed and requires both strength and plasticity of the film. One way to achieve a satisfactory plasticity is to add one or more plasticizers. Plasticizers might be defined as small low molecular weight, non-volatile compounds added to polymers to reduce brittleness, impart flexibility, and enhance toughness for films. In general, the optimal type and concentration of plasticizer(s) depends on a range of factors, such as the type and concentration of polymer(s). The type and concentration of API, as well as its state (i.e., dissolved or suspended inside the film), may also have an impact when selecting optimal type and concentration of plasticizer(s), at least if the substance constitutes a significant fraction of the finished film e.g., more than 10 wt %.

In one embodiment, the unit dosage form of the present invention comprises an API, a film-forming polymer and one or more plasticizer(s).

In one embodiment, the plasticizer is selected from the group consisting of glycerol; glycerol monoacetate; citric acid and esters thereof such as triethyl citrate (TEC); diethylene glycol; ethylene glycol; fatty acid esters; PEG, such as PEG 400, PEG 600 or PEG 4000; polyethylene-propylene glycols; propylene glycol; phthalic acid; polyalkylene oxides; sorbitol, triacetin and xylitol. In one embodiment, the plasticizer is glycerol. In one embodiment, the plasticizer is TEC. In one embodiment, the plasticizer is poloxamer 407. Poloxamer 407 is a triblock copolymer consisting of a central hydrophobic block of polypropylene glycol flanked by two hydrophilic blocks of polyethylene glycol (PEG). The approximate lengths of the two PEG blocks is 101 repeat units, while the approximate length of the propylene glycol block is 56 repeat units. Thus, poloxamer 407 is a polypropylene glycol-polyethylene glycol copolymer. Poloxamer 407 is also known as Pluronic F-127, Synperonic PE/F 127 and Kolliphor P 407. In one embodiment, the plasticizer is Kollicoat IR. Kollicoat IR is a polymer comprising about 75% polyvinyl alcohol units and about 25% polyethylene glycol units, and optionally about 0.3% colloidal anhydrous silica. Thus, Kollicoat IR is a polyvinyl alcohol-polyethylene glycol copolymer. In one embodiment, the plasticizer is selected from the group consisting of glycerol; glycerol monoacetate; citric acid and esters thereof such as triethyl citrate (TEC); diethylene glycol; ethylene glycol; fatty acid esters; PEG, such as PEG 400, PEG 600 or PEG 4000; polyethylene-propylene glycols; propylene glycol; phthalic acid; polyalkylene oxides; sorbitol, triacetin and xylitol.

Some film-forming polymer contains molecular elements that will contribute to the plasticity of the final film and may sometimes render it unnecessary to add a separate plasticizer. One such polymer is Kollicoat IR, in which it is the polyethylene glycol units that are believed to provide the plasticity.

In one embodiment, the unit dosage form does not comprise any plasticizer.

In one embodiment, the unit dosage form does not comprise any plasticizer and the film-forming polymer is Kollicoat IR.

In one embodiment, the unit dosage form does not comprise any plasticizer and the film-forming polymer is HPMC 2528. In one embodiment, the unit dosage form does not comprise any plasticizer and the film-forming polymer is a HPMC with the trade name Affinisol HPMC HME 15 LV. In one embodiment, the unit dosage form does not comprise any plasticizer and the film-forming polymer is a HPMC with the trade name Affinisol HPMC HME 100 LV. In one embodiment, the unit dosage form does not comprise any plasticizer and the film-forming polymer is a HPMC with 23-27% methoxy content and 26-30% hydroxypropoxy content.

In one embodiment, the unit dosage form does not comprise any plasticizer. In one embodiment, the unit dosage form does not comprise any plasticizer and the film-forming polymer is HPMC 1828. In one embodiment, the unit dosage form does not comprise any plasticizer and the film-forming polymer is a HPMC with 16-20% methoxy content and 26-30% hydroxypropoxy content.

In one embodiment, the unit dosage form does not comprise any plasticizer and the film-forming polymer is HPC.

In one embodiment, the unit dosage form comprises 3 wt % plasticizer. In one embodiment, the unit dosage form comprises more than 3 wt % but less than 5 wt % plasticizer. In one embodiment, the unit dosage form comprises at least 3 wt % plasticizer, such as at least 5 wt %, such as at least 10 wt %, such as at least 30 wt % plasticizer. In one embodiment, the unit dosage form comprises no more than 30 wt % plasticizer, such as no more than 20 wt %, such as no more than 15 wt %, such as no more than 10 wt % plasticizer. In one embodiment, the unit dosage form comprises 3 to 35 wt % plasticizer, such as 4 to 10 wt %, such as about 5 wt % plasticizer.

In one embodiment, the unit dosage form comprises a combination of two plasticizers, for which the total concentration is equal to any of the levels presented above. In one embodiment, said two plasticizers are selected from the group consisting of glycerol, TEC, poloxamer 407 and Kollicoat IR, such as glycerol and TEC, or glycerol and poloxamer 407, or glycerol and Kollicoat IR, or TEC and poloxamer 407, or TEC and Kollicoat IR, or poloxamer 407 and Kollicoat IR.

### Additives

In one embodiment, the unit dosage form further comprises one or more additives, for example a colorant, such as a pigment, a taste masking agent, and/or flavouring agents.

In one embodiment, the unit dosage form comprises an API, one or more film-forming polymers, one or more flavouring agents and a pigment but no other additives or excipients. In one embodiment, the unit dosage form comprises an API, one or more film-forming polymers, a pigment but no other additives or excipients.

In one embodiment, the pigment is selected from the group consisting of yellow iron oxide, red iron oxide and black iron oxide.

In one embodiment, the flavour(s) is such that it mitigates the sensation of a bitter emanating from the API added to the film.

In one embodiment, the unit dosage form comprises at least 0.2 wt % pigment, such as at least 0.5 wt %, such as at least 1 wt % pigment. In one embodiment, the unit dosage form comprises no more than 10 wt % pigment, such as no more than 5 wt %, such as no more than 2 wt %, such as no more than 1 wt % pigment. In one embodiment, the unit dosage form comprises 0.5 to 5 wt % pigment, such as about 1 wt % pigment.

In one embodiment, the unit dosage form comprises at least 1 wt % flavour(s), such as at least 2 wt %, such as at least 5 wt %. In one embodiment, the unit dosage form comprises no more than 20 wt % flavour(s), such as no more than 10 wt % flavour(s).

### Total Composition

In one embodiment, the unit dosage form comprises 15 to 45 wt % API and 35 to 80 wt % film-forming polymer, such as 25 to 40 wt % API and 45 to 70 wt % film-forming polymer.

In one embodiment, the unit dosage form comprises 15 to 45 wt % API, 35 to 80 wt % film-forming polymer and 3 to 35 wt % plasticizer.

In one embodiment, the unit dosage form comprises 15 to 45 wt % midazolam, 35 to 80 wt % HPMC and 3 to 15 wt % glycerol. In one embodiment, the unit dosage form comprises 30 to 45 wt % midazolam, 50 to 60 wt % HPMC and 3 to 8 wt % glycerol. In one embodiment, the unit dosage form comprises about 31 to 35 wt % midazolam, 60 to 64 wt % HPMC and 3 to 7 wt % glycerol. In one embodiment, the unit dosage form comprises about 33 wt % midazolam, 61 wt % HPMC and 5 wt % glycerol. In one embodiment, the unit dosage form comprises about 33 wt % midazolam, about 63 wt % HPMC and 4 wt % glycerol.

In one embodiment, the unit dosage form comprises 33 wt % midazolam or a pharmaceutically acceptable salt thereof, 61 wt % HPMC, 5 wt % glycerol, 1 wt % yellow iron oxide.

In one embodiment, the unit dosage form comprises 33 wt % midazolam or a pharmaceutically acceptable salt thereof, 66 wt % HPMC and 1 wt % yellow iron oxide.

In one embodiment, the unit dosage form comprises 33-40 wt % midazolam or a pharmaceutically acceptable salt thereof, 59-66 wt % HPMC and 1 wt % yellow iron oxide.

The amounts of the various components of the unit dosage form or the film are sometimes given as wt %. In such cases, the sum of the wt % of the components does not exceed 100 wt %.

### Size

One key feature for oral films is that they are thin, for example less than 250 μm, in order to be mechanically flexible and also to avoid a very slow dissolution rate.

Usually, the thinner the film, the faster the dissolution, for one and the same API and composition. For many film-forming polymers, including most variants of HPMC, a thickness of about 40 μm or less is likely to result in a very fast or even instantaneous dissolution, in vitro as well as in vivo. Likewise, for many film-forming polymers, including most variants of HPMC, a thickness of 250 μm or more is likely to result in a very slow dissolution, e.g., that less than 85% of the API has been dissolved within 10 minutes in the USP Dissolution Apparatus 2—Paddle. It should however be understood that the dissolution rate is also largely dependent on the film-forming polymer as well as on other aspects of the composition (e.g., the use of disintegration agents) and hence there is no universal relation between thickness and dissolution rate.

Another key feature is that films should have a feasible area that fits into the oral cavity surfaces, e.g., <5 cm², yet large enough for convenient handling by the patient or the person helping the patient, e.g., >2 cm².

In one embodiment, the oral film according to this invention is 50 to 150 μm thick, such as 60 to 120 μm thick, such as 70 to 110 μm thick, such as 80 to 100 μm thick.

In one embodiment, the oral film according to this invention is 40 to 100 μm thick, such as 50 to 90 μm thick, such as 60 to 80 μm thick.

The thickness of an oral film is often measured and defined by coat weight, rather than being measured as an actual thickness and presented in μm. Coat weight is the weight of the dry film per unit area and is usually presented as g/m². If the density of the dry film is 1 g/cm³, the numerical values of thickness in μm will equal that of coat weight in g/m².

In one embodiment, the unit dosage form is rectangular and has a dimension of X×Y×Z, wherein X is in the range of 0.5 to 5 cm; Y is in the range of 0.5 to 5 cm; and Z is in the range of 15 to 150 μm.

In one embodiment, X is at least 0.5 cm, such as at least 1 cm, such as at least 1.5 cm, such as at least 2 cm. In one embodiment, X is no more than 5 cm, such as no more than 4.5 cm, such as no more than 4 cm, such as no more than 3.5 cm, such as no more than 3 cm. In one embodiment, X is in the range of 0.5 to 5 cm, such as in the range of 1 to 3 cm, for example in the range of 1 to 2 cm.

In one embodiment, Y is at least 0.5 cm, such as at least 1 cm, such as at least 1.5 cm, such as at least 2 cm. In one embodiment, Y is no more than 5 cm, such as no more than 4.5 cm, such as no more than 4 cm, such as no more than 3.5 cm, such as no more than 3 cm. In one embodiment, Y is in the range of 0.5 to 5 cm, such as in the range of 1 to 3 cm, for example in the range of 2 to 3 cm.

In one embodiment, Z is at least 5 μm, such as at least 25 μm, such as at least 50 μm, such as at least 75 μm, such as at least 100 μm. In one embodiment, Z is no more than 1 mm, such as no more than 750 μm, such as no more than 500 μm, such as no more than 250 μm, such as no more than 125 μm. In one embodiment, Z is in the range of 5 μm to 750 μm, such as in the range of 30 to 150 μm, such as 50 to 120 μm, such as 70 to 110 μm.

In one embodiment, X is in the range of 0.5 to 5 cm; Y is on the range of 0.5 to 5 cm; and Z is in the range of 30 μm to 150 μm. In one embodiment, X is in the range of 1 to 3 cm; Y is on the range of 1 to 3 cm; and Z is in the range of 50 μm to 150 μm.

In one embodiment, the unit dosage form is rectangular and has a dimension of about 1.5 cm×2.5 cm×90 μm.

In one embodiment, the unit dosage form is rectangular and has an area, i.e., X×Y, of 1 to 6 cm², such as 1.5 to 5 cm², such as 3 to 4.5 cm², such as 3.5 to 4 cm².

In one embodiment has a coat weight of 50 to 150 g/m², such as 75 to 125 g/m², such as 80 to 110 g/m², such as about 90 g/m².

In one embodiment, the unit dosage form is rectangular and has an area of 1 to 6 cm², such as 1.5 to 5 cm², such as 3 to 4.5 cm², such as 3.5 to 4 cm², and is 30 to 150 μm thick, such as 50 to 120 μm thick, such as 70 to 110 μm thick.

In one embodiment, the unit dosage form is rectangular and has an area, i.e., X×Y, of 1 to 6 cm², such as 1.5 to 5 cm², such as 3 to 4.5 cm², such as 3.5 to 4 cm², and a coat weight of 50 to 150 g/m², such as 75 to 125 g/m², such as 80 to 110 g/m², such as about 90 g/m².

In one embodiment, the unit dosage form is rectangular and can be described with any of the X×Y×Z descriptions above but has fully rounded corners i.e., the ends are semi-circular. Such form is also referred to as a stadium form of a rectangle.

In one embodiment, the unit dosage form is rectangular and can be described with any of the X×Y×Z descriptions above but all corners are rounded yet not forming the stadium form of a rectangle.

In one embodiment, the unit dosage form is oval and has an area of 2 to 5 cm², such as 2.5 to 4.5 cm².

In one embodiment, the unit dosage form is circular and has an area of 2 to 5 cm², such as 2.5 to 4.5 cm². In one embodiment, the unit dosage form is circular and has a radius of 8 mm to 13 mm.

In one embodiment, the unit dosage form consists of a single layer.

### Manufacturing Process

There are several principles for preparing and manufacturing oral films, for example as described in U.S. Pat. No. 11,173,114 B1 and by Kathpalia and Gupte 2013. The most common principle is probably the solvent casting method which can be described as:

a) Mixing the API and one or more film-forming polymers in one or more volatile solvents to provide a wet mix. Either the API is added firstly and a homogeneous mix is obtained before the polymer(s) is added, or the other way around, or API and polymer being added at the same time. Other excipients are also added at various timepoints within this step. Each one of the API and these other excipients can be either dissolved or suspended in the resulting wet mix, but the film-forming polymer(s) should in most cases be dissolved. The wet mix is typically made in a batchwise manner, and hence the wet mix batch size will be determining the batch size of the oral film manufacturing.

b) Casting the wet mix obtained in step a) to provide a wet film. The wet mix is cast onto an inert release liner, with a typical thickness within 100-1000 micrometer. At lab scale, this can be made in a batchwise manner, using a sheet of the release liner. In pilot and large scale however, this is usually made in a continuous manner, with the release liner moving away from the point of coating.

c) Drying said wet film to obtain the final, dry film. The wet film is dried until an essentially solvent-free, dry film is obtained, with typical thickness within 20-200 micrometer, said thickness being a function of the thickness of the wet film and the dry content of the wet mix. In pilot and large scale, this step is made in a continuous manner, with the wet film on release liner typically moving through some kind of heated drying tunnel and eventually, after drying, being rolled up onto one or more mother rolls. These mother rolls can optionally be cut into daughter rolls with less width of the dry film.

d) Converting said film into unit dosages by cutting it into feasible sizes and packaging these into primary containers. The dry film is rolled off its mother or daughter roll and cut into individual film units which are packaged into air- and water-tight pouches which become the primary containers of the unit dosage form. This step d) is often referred to as the “converting step”. Step d) is not “solvent casting method” per se, but it is a necessary subsequent step.

As for excipients other than the film-forming polymer in step a), several excipients can be imagined: plasticizers (e.g., glycerol), fillers, colorants, flavours, taste maskers, disintegration agents, dissolution agents, solubilizing agents, etc.

Water is the most commonly used solvent, but non-aqueous solvents, e.g., ethanol, are sometimes used as co-solvents with the aim of aiding to dissolving the API in the wet mix and keep it dissolved. Such non-aqueous solvents, e.g., ethanol, can also be used as the sole solvent, or as co-solvents together with one or more other non-aqueous solvents. This is further explained below.

There are several variants of the process described above, for example, the order in which ingredients are added in step a), or that multilayer films are made, or that the API is added in the form of a solid, particulate intermediate product that has already been mixed and pre-processed with other excipients (for example, into a solid solution intermediate material that is pulverized).

The difference in step a) between dissolved API and suspended API is essential, because it will determine the final state of the API inside the finished dry film and thereby determine critical attributes such as dissolution rates and stability. As used herein, the term “suspended API” refers to an API in a solid, particulate state inside the film. Preferably, in that case, the solid state and polymorph are the same as for the API starting material used for the manufacturing. As used herein, the term “dissolved API” refers to an API that is not in a solid, particulate state inside the film but instead dissolved which can also be referred to as “solid solution” or “molecular dispersion. Preferably, in that case, the API should stay in that dissolved state i.e., not precipitating into solid particles inside the film matrix during manufacturing or storage.

In one embodiment of the present invention, the solvent casting method as described above in step a) to c) is used, and with each of the following specifications: a non-aqueous solvent is used; the polymer is dissolved in the wet mix; the API is not dissolved in the wet mix but suspended; the API remains suspended throughout the manufacturing; the film is a monolayer film; the API has not been pre-processed into an intermediate that also contains other ingredients. In one embodiment, there are no other ingredients than the API, a film-forming polymer, a pigment and a flavour. In one embodiment, the API is midazolam hydrochloride, the film-forming polymer is HPMC, and the solvent is ethyl acetate.

In one embodiment, the polymer is added before the API and a homogeneous wet mix with that polymer in dissolved state is achieved before the API is added. In one embodiment, the API is added before the polymer and a homogeneous wet mix with the API in suspended state is made before the polymer is added.

In one embodiment, the wet film thickness is 300 to 800 μm, such as 400 to 700 μm, such as 550 to 650 μm.

In one embodiment, the wet film is dried until the residual solvent content is no more than 5 wt %, such as no more than 4 wt %, such as no more than 3 wt % such as no more than 2 wt %, such as no more than 1 wt %.

In one embodiment, the drying is performed at 40 to 120° C., such as 50 to 110° C., such as 60 to 100° C. In one embodiment, the drying is performed by using several temperature zones, for example, first moving the film in a zone with 80° C., the moving it into another zone with 95° C., etc. In one embodiment, the intermediate layer is an inert, strong, flexible polymer material such as PET.

In one embodiment, the process is used for preparing a film comprising midazolam as API. In one embodiment, midazolam is added as the midazolam hydrochloride salt. In another embodiment, midazolam is added as midazolam maleate.

In one embodiment, the intermediate layer is an inert, strong, flexible polymer material such as PET.

In one embodiment, the process is used for preparing a film comprising midazolam as API. In one embodiment, midazolam is added as the midazolam hydrochloride salt. In another embodiment, midazolam is added as midazolam maleate.

In one aspect, the present invention relates to a film or a unit dosage form obtainable by the process as described herein.

### Process Solvents

The choice of solvent in the manufacturing process is essential. The solvent should be volatile, it should preferably be non-toxic and non-hazardous, and it should dissolve the film-forming polymer(s). Virtually all film-forming polymers used for making oral films are water-soluble, because normally the eventual fate of the film is to be dissolved in the aqueous environment in the oral cavity in vivo.

Obviously, when taking these criteria into consideration, water is the most common process solvent being used in solvent casting preparation of oral films.

Many API molecules are however sparingly soluble in water, and if water is the sole process solvent when oral films are made with such APIs, the added API may not dissolve in the wet mix, and the eventual dry film will then contain the API wholly or predominately in the suspended state. This may be the intention of those designing the film and does not have to be detrimental to the in vivo absorption and clinical effect of the oral film.

However, in some cases, it strongly desired that the eventual dry film should contain the API in dissolved form, for example with the aim to achieve a satisfactory in vivo dissolution rate and subsequent absorption and clinical effect. In that case, the API must be dissolved in the wet mix, and stay dissolved throughout the drying step. Using water as the sole solvent may not be sufficient for that, and the approach will be to either use one or more non-aqueous solvents as cosolvents alongside the water, or to replace the water with one or more non-aqueous solvents.

Hence, there are currently three main approaches with regard to the process solvent:
Using water as solvent, with the intention that the API will be suspended in the wet mix, and in the subsequent dry film and remaining so during storage.
Using water as solvent, with the intention that the API will be dissolved in the wet mix, and in the subsequent dry film and remaining so during storage.
Using non-aqueous co-solvents/solvents, with the intention that the API will dissolve in the wet mix, and in the subsequent dry film and remaining so during storage.

Alternative or supplementary ways to keep the API in the dissolved state may also be employed, such as pH adjustment or the use of solubilizing agents such as surfactants or cyclodextrin.

Below, non-aqueous solvents and non-aqueous co-solvents are sometimes collectively referred to as “solvents”.

Such other solvents have to be volatile and should preferably be non-toxic because there may be residual amounts in the finished product. They should preferably also be non-hazardous for use in a manufacturing facility, although whether a solvent is hazardous or not depends on the process equipment and facility. The term volatile herein, when applied to non-aqueous solvents, means a solvent that has a boiling point below 100±5° C. at normal atmospheric pressure, a flash point below room temperature and a vapor pressure above 1.5 kPa, either when being the sole solvent or being a co-solvent in a mix together with one or more other solvents.

Examples of solvents with potential use in the solvent casting method described above are: acetic acid; acetone; acetonitrile; 1-butanol; 2-butanol; butyl acetate; dimethyl sulfoxide (DMSO); N, N-dimethyl-acetamide (DMA); N, N-dimethyl-formamide (DMF); 1,4-dioxane; d-limonene; ethanol; ethyl acetate; formic acid; isobutyl acetate; isopropanol; isopropyl acetate; methanol; methyl acetate; methyl ethyl ketone; methylene chloride; 3-methyl-1-butanol; 2-methyl-1-propanol; 1-methyl-2-pyrrolidone; 1-pentanol; 1-propanol; 2-propanol; propyl acetate; tetrahydrofuran (THF); and triethylamine.

The choice of solvents, when using the solvent casting method for the intention to have the API in the dissolved state inside the film, will mainly depend on the API. For most API molecules, there are public information about which solvents that are feasible, although there is rarely information of an API's solubility in each and all of the above listed solvents. It should also be noted that the solvent must also allow the film-forming polymer to dissolve in the wet mix.

It can thus be understood that it is not a trivial task to identify non-aqueous solvents for use in the solvent casting method of making oral films.

The inventors behind the present invention have now conceived a new principle for the identification and selection of feasible non-aqueous solvents for use in the solvent casting method. That principle applies to APIs that are readily soluble in water but for which there is a desire that the API should not be in the dissolved state inside the finished, dry film. The principle is to select a non-aqueous solvent in which the film-forming polymer is soluble but the API is not. This is different from the current principle to employ non-aqueous solvents with the purpose to aid the dissolution of the API in the wet mix.

One reason for not wanting the API to be in the dissolved state inside the film, but instead being in suspended state, is to achieve a better physical and/or chemical stability of the film product.

In one embodiment of the present invention, the API is thus readily soluble in water but intended to be suspended inside the film. In one embodiment, a non-aqueous solvent is used for that purpose which is able to dissolve the film-forming polymer but not the API. In one embodiment, that API is midazolam hydrochloride or midazolam maleate.

In one embodiment, the solvent has been identified and selected by a screening procedure with the following elements:

1) A number of non-toxic, volatile solvents are screened for their ability to dissolve the API.

2) Those solvents that are not dissolving the API are selected. The criterium used for “not dissolving the API” is: in an API:solvent mix with 5:95 weight ratio, not more than 5 wt % of the added API should be dissolved after stirring for 2 hours at room temperature.

3) A number of film-forming polymers are screened for their ability to be dissolved in the solvent(s) selected in step 2. The criterium used for “being dissolved in the solvent(s)” is: in an polymer:solvent mix with 15:85 ratio, not more than 5 wt % of the added polymer should remain non-dissolved after stirring for 2 hours at room temperature.

4) Finally, selecting the one or more solvent selected in step 2) that dissolves one or more of the selected polymers in step 3).

In one embodiment, the one or more solvent used belong to the group consisting of acetone, 1-butanol, 2-butanol, butyl acetate, ethyl acetate, isobutyl acetate, isopropyl acetate, methyl acetate, methyl ethyl ketone, 2-methyl-1-propanol, 1-pentanol, 1-propanol, 2-propanol, propyl acetate and triethylamine.

In one embodiment, the one or more solvent used belong to the group consisting of acetone, ethyl acetate, isobutyl acetate, methyl acetate, 1-pentanol and 2-propanol. In one embodiment, the one or more solvent used belong to the group consisting of acetone, ethyl acetate, isobutyl acetate and methyl acetate.

In one preferred embodiment, the solvent used is ethyl acetate.

In one preferred embodiment, the solvent used is ethyl acetate and the API is midazolam hydrochloride or midazolam maleate.

In one embodiment, the solvent used is ethyl acetate, the API is midazolam hydrochloride or midazolam maleate, and the manufacturing process used is described by step a)-d) in the section Manufacturing process above.

In one embodiment, the solvent used is ethyl acetate, the API is midazolam hydrochloride or midazolam maleate, and the amount of residual ethyl acetate is not less than 1 000 ppm (parts per million), such as not less than 5 000 ppm, such that not less than 10 000 ppm, such as less not than 25 000 ppm.

### Biological Activity and Medical Use

In one aspect, the present invention relates to a unit dosage form as described herein for use as a medicament.

In one aspect, the present invention relates to a unit dosage form as described herein for use in the acute treatment of seizures in a subject. In one embodiment, the seizures are caused by epilepsy or another disease or condition that may cause seizures.

In one embodiment, the subject is suffering from epilepsy or another disease or condition that may cause seizures.

In one embodiment, the epilepsy is generalised epilepsy or partial epilepsy.

In one embodiment, the disease or condition that may cause seizures is selected from the group consisting of fever caused by malaria, fever of other causes, poisoning, tetanus, brain tumours, Lennox-Gastaut syndrome, tuberous sclerosis complex and Dravet syndrome.

In one embodiment, the seizures are selected from the group consisting of cluster seizures, seizure convulsions, convulsions, spasms, prolonged acute convulsive seizures, stereotypic episodes of frequent seizure activity that are distinct from a patient's usual seizure pattern, status epilepticus and convulsive refractory status epilepticus.

In one embodiment, the seizures are ongoing, acute seizures.

In one embodiment, the unit dosage form used for such treatment of various seizures is an oral film which is buccally applied. Such films are often referred to as “buccal films”.

In one embodiment, this unit dosage form is being used to treat patients with typically exhibits a behaviour—when suffering from a seizure—which means that they are drooling and/or swallowing saliva. In that embodiment, the loss of API dose due to that behaviour is reduced by the use of this unit dosage form, if compared with being treated with the same dose in the form of a buccal or oromucosal solution, or with a film that has instantaneous dissolution.

In one aspect, the present invention relates to a unit dosage form as described herein for use in moderate sedation before diagnostic, therapeutic or surgical procedures or pre-sedation before anaesthesia. In one embodiment, the diagnostic, therapeutic or surgical procedures include but it limited to these kinds of procedures within odontology.

In one aspect, the present invention relates to use of a unit dosage form as described herein in moderate sedation before diagnostic, therapeutic or surgical procedures or pre-sedation before anaesthesia.

In one embodiment, the unit dosage form used for such moderate or pre-sedation is an oral film which is applied onto the tongue. Such films are often referred to as “orodispersible films” or “ODF”.

In one embodiment, the unit dosage form used for such moderate or pre-sedation is an oral film that is sublingually applied. Such films are often referred to as “sublingual films”.

In one embodiment, the unit dosage form used for such moderate or pre-sedation is an oral film that is buccally applied. Such films are often referred to as “buccal films”.

In one embodiment, the unit dosage form is being used to treat patients with typically exhibits a behaviour—when needing moderate sedation or pre-sedation—which means that they are drooling and/or deliberately spitting out saliva. In that embodiment, the loss of API dose due to that behaviour is reduced by the use of this unit dosage form, if compared with being treated with the same dose in the form of an oral solution or syrup, or with a film that has instantaneous dissolution.

In one embodiment, the subject is a mammal, such as a human. In one embodiment, the subject is a dog, a horse or a cat.

In one aspect, the present invention relates to a method of treating seizures in a subject, said method comprising administering the unit dosage form as described herein.

In one aspect, the present invention relates to the use of the unit dosage form as described herein in the manufacture of a medicament for use in the acute treatment of seizures in a subject.

### Bioavailability

In one embodiment, the unit dosage form is an oral film used for buccal administration. For said use, the oral film may be categorized as a “buccal film”. Example 22 describes a human bioavailability study of an oral film comprising 10 mg midazolam and having a moderately high dissolution rate. In the study the film is buccally administered, with the intention to compare its buccal absorption (i.e., buccal-transmucosal) with that of a commercially available buccal solution (BUCCOLAM, with 10 mg midazolam). The study subjects were instructed not to swallow the products or any saliva, but they were allowed to empty their mouth from excessive saliva at 5 minutes and at 10 minutes after the administration of the study products. It was found that, with those study conditions, the unit dosage form of the present invention had a higher bioavailability than that of the commercial buccal solution.

Thus, in one embodiment, the total drug exposure across time, for example measured as area under the curve (AUC) of the API, such as midazolam, is higher for the unit dosage form of the present invention than for a corresponding buccal solution of the API. In one embodiment, AUC is at least 25% higher for the unit dosage form of the present invention compared to a buccal solution with the same dose, such as at least 50% higher, such as at least 75% higher.

Thus, in one embodiment, the maximum serum concentration, for example measured as Cₘₐₓ, achieved for the API, such as midazolam, is higher for the unit dosage form of the present invention than for a corresponding buccal solution of the API. In one embodiment, Cₘₐₓ is at least 25% higher for the unit dosage form of the present invention compared to a buccal solution with the same dose, such as at least 50% higher, such as at least 75% higher.

In one embodiment, the unit dosage form is an oral film used for oral administration, i.e., onto the tongue. For said use, the oral film may be categorized as a “orodispersible film”. Example 23 describes a human bioavailability study of an oral film comprising 10 mg midazolam and having a moderately high dissolution rate. In the study the film is orally administered with the intention to compare its oral absorption (i.e., oral-gastrointestinal) with that of a commercially available oral solution (Midazolam Hydrochloride Syrup 2 mg/mL). Contrary to Example 22, the study subjects in this study were not instructed not to swallow the products or any saliva, but they were instructed not to spit out any product or saliva or otherwise empty theory mouths from excess saliva. (Such instructions are normal or self-evident when oral products are being studied). It was found that, with those study conditions, the unit dosage form of the present invention had a higher bioavailability than that of the commercial oral solution. It was also found, that the interindividual variability was lower for the oral film than for the oral solution.

The word oral solution refers to a pharmaceutical dosage form in which one or more APIs are dissolved and which is liquid and intended to be swallowed by the patient. The word “oral syrup” is also applicable. While every such product is unique and may for example differ with regard to viscosity or sweeteners, there is no implicit or explicit difference in the meaning of the word “oral syrup” vs the word “oral solution”.

Thus, in one embodiment, the total drug exposure across time, for example measured as area under the curve (AUC) of the API, such as midazolam, is higher for the unit dosage form of the present invention than for an oral solution of the API with the same dose. In one embodiment, AUC is at least 25% higher for the unit dosage form of the present invention compared to an oral solution of the API with the same dose, such as at least 50% higher, such as at least 75% higher.

Thus, in one embodiment, the maximum serum concentration, for example measured as Cₘₐₓ, achieved for the API, such as midazolam, is higher for the unit dosage form of the present invention than for an oral solution of the API with the same dose. In one embodiment, Cₘₐₓ is at least 25% higher for the unit dosage form of the present invention compared to an oral solution of the API with the same dose, such as at least 50% higher, such as at least 75% higher.

Thus, in one embodiment, the intraindividual variability of AUC and/or Cₘₐₓ is lower after the administration of the unit dosage form of the present invention than after an oral solution of the API with the same dose.

It is well-known that after oral administration of midazolam and several other benzodiazepines, a double-peak phenomenon occurs for the plasma curve over time (Belle et al. 2002). Firstly, one peak of the plasma concentration over time occurs, with a magnitude and at a timepoint that may be similar to what could be expected without the double peak phenomenon. Secondly, another peak arises, after for example 0.5-1 hours, with a magnitude that may be comparable to the first peak. This is believed to be a result of reduced gastric motility caused by muscle relaxant effect of benzodiazepines.

In one embodiment, the unit dosage form of the present invention does not produce such double-peak phenomenon after being orally administered.

### Items

1. A unit dosage form in the form of an oral film comprising an active pharmaceutical ingredient (API) and one or more film-forming polymers.
2. The unit dosage form according to item 1, wherein the API is capable of providing relief from ongoing seizures.
3. The unit dosage form according to any one of the preceding items, wherein the API is an anticonvulsant.
4. The unit dosage form according to any one of the preceding items, wherein the API has a sedative effect.
5. The unit dosage form according to item 4, wherein the API is useful in moderate sedation before diagnostic, therapeutic or surgical procedures or pre-sedation before anaesthesia.
6. The unit dosage form according to any one of the preceding items, wherein the API is selected from the group consisting of benzodiazepines and benzodiazepine-like substances.
7. The unit dosage form according to any one of the preceding items, wherein the API is selected from the group consisting of midazolam, diazepam, alprazolam, brotizolam, cinolazepam, clizolam, clobazam, clonazepam, clonazolam, clorazepate, cloxazolam, diclazepam, estazolam, flubromezepine, flunitrazepam, flurazepam, flutoprazepam, kvazepam, lorazepam, loprazolam, lormetazolam, metizolam, nitrazepam, oxazepam, phenazepam, temazolam, triazolam and pharmaceutically acceptable salts thereof.
8. The unit dosage form according to any one of the preceding items, wherein the API is midazolam, diazepam, clobazam, clonazepam, lorazepam or a pharmaceutically acceptable salt thereof.
9. The unit dosage form according to any one of the preceding items, wherein the API is midazolam, diazepam, or a pharmaceutically acceptable salt thereof.
10. The unit dosage form according to any one of the preceding items, wherein the API is midazolam or a pharmaceutically acceptable salt thereof.
11. The unit dosage form according to any one of the preceding items, wherein the API is midazolam hydrochloride.
12. The unit dosage form according to any one of items 1 to 10, wherein the API is midazolam maleate.
13. The unit dosage form according to any one of the preceding items, wherein the unit dosage form comprises at least 2.5 mg of API, such as at least 5 mg, such as at least 10 mg of API.
14. The unit dosage form according to any one of the preceding items, wherein the unit dosage form comprises no more than 20 mg of API, such as no more than 15 mg, such as no more than 10 mg of API.
15. The unit dosage form according to any one of the preceding items, wherein the unit dosage form comprises 2.5 to 20 mg of API, such as 5 to 15 mg, such about 10 mg, such as about 7.5 mg, such as about 5 mg of API.
16. The unit dosage form according to any one of the preceding items, wherein the unit dosage form comprises about 10 mg API.
17. The unit dosage form according to any one of the preceding items, wherein the API is midazolam or a pharmaceutically acceptable salt thereof, and the unit dosage form comprises about 10 mg midazolam (defined as the base).
18. The unit dosage form according to any one of items 1 to 15 wherein, the unit dosage form comprises about 7.5 mg API (defined as the base).
19. The unit dosage form according to any one of items 1 to 15, wherein the API is midazolam or a pharmaceutically acceptable salt thereof, and the unit dosage form comprises about 7.5 mg midazolam (defined as the base).
20. The unit dosage form according to any one of items 1 to 15, wherein the unit dosage form comprises about 5 mg API.
21. The unit dosage form according to any one of items 1 to 15, wherein the API is midazolam or a pharmaceutically acceptable salt thereof, and the unit dosage form comprises about 5 mg midazolam (defined as the base).
22. The unit dosage form according to any one of the preceding items, wherein the API concentration is at least 10 wt %, such as at least 15 wt %, such as at least 20 wt %, such as at least 25 wt %, such as at least 30 wt %, such as at least 40 wt %.
23. The unit dosage form according to any one of the preceding items, wherein the API concentration is no more than 80 wt %, such as no more than 70 wt %, such as no more than 60 wt %, such as no more than 50 wt %, such as no more than 40 wt %.
24. The unit dosage form according to any one of the preceding items, wherein the API concentration is 10 to 60 wt %, such as 20 to 50 wt %, such as 25 to 50 wt %, such as 30 to 40 wt %.
25. The unit dosage form according to any one of the preceding items, wherein the API is present in the form of solid, suspended particles inside the film, which have not undergone any chemical, physical or morphological changes compared with the original API that was added during in the manufacturing.
26. The unit dosage form according to any one of the preceding items, wherein the API is suspended inside the film of the unit dosage form.
27. The unit dosage form according to any one of the preceding items, wherein at least 80 wt %, such as at least 90 wt %, such as at least 95 wt %, such as at least 98 wt % of the total amount of API is suspended in the film of the unit dosage form.
28. The unit dosage form according to any one of the preceding items, wherein the concentration of API in the film is in the range of 15 to 35 wt % and at least 60 wt %, such as at least 70 wt %, such as at least 80 wt %, such as at least 90 wt %, such as at least 95 wt %, such as at least 98 wt % of the API is suspended in the film of the unit dosage form.
29. The unit dosage form according to any one of the preceding items, wherein the API is midazolam or a pharmaceutically acceptable salt thereof and the unit dosage form has a moderately high dissolution rate.
30. The unit dosage form according to any one of the preceding items, wherein the unit dosage form comprises two or more active pharmaceutical ingredients.
31. The unit dosage form according to any one of the preceding items, wherein the film-forming polymer is soluble in ethyl acetate.
32. The unit dosage form according to any one the preceding items, wherein the film-forming polymer is selected from the group consisting of:
   i. HPMC 2528;
   ii. HPC;
   iii. hypromellose acetate succinate;
   iv. methacrylic acid-methyl acrylate copolymers; and
   v. polyvinyl caprolactam-polyvinyl acetate-polyethylene glycol graft copolymer.
33. The unit dosage form according to any one of items 1 to 31, wherein the film-forming polymer is selected from the group consisting of acrylates, alginates, carrageenan, cellulose derivatives, chitosan, collagen, dendritic polymers, gelatine, gum, hyaluronic acid, maltodextrin, pectin, polyethylene glycol, polyethylene oxide, polylactic acid and derivatives or copolymers thereof, polysaccharides, pullulan, polyvinylpyrrolidone, scleroglucan, starch, starch derivatives, and polyvinyl alcohol.
34. The unit dosage form according to according to any one of items 1 to 31, wherein the film-forming polymer is hypromellose (HPMC).
35. The unit dosage form according to according to item 34, wherein the film-forming polymer is a HPMC with a viscosity of at least 1 mPas, such as about 3 mPas, such as about 4 to 5 mPas, such as about 5 mPas, such as at least 10 mPas, such as about 15 mPas, such as about 50 mPas.
36. The unit dosage form according to any one of 34 or 35, wherein the film-forming polymer is a HPMC with a viscosity of no more than 100 000 mPas, such as no more than 15 000 mPas, such as no more than 5 000 mPas, such as no more than 1 000 mPas, such as no more than 500 mPas, such as no more than 100 mPas.
37. The unit dosage form according to any one of items 34 to 36, wherein the film-forming polymer is HPMC with a substitution type of about 25 to 31% for hydroxypropoxy, such as 26 to 30%, such as or 27 to 29%, such as 28% for hydroxypropoxy.
38. The unit dosage form according to any one of items 34 to 36, wherein the film-forming polymer is a HPMC with a substitution type of about 22 to 28% for methoxy and about 25 to 31% for hydroxypropoxy, such as 23 to 27% or 24 to 26% for methoxy and such as 26 to 30% or 27 to 29% for hydroxypropoxy.
39. The unit dosage form according to item any one of items 34 to 38, wherein the HPMC is of substitution type 2528.
40. The unit dosage form according to any one of items 34 to 39, wherein the film-forming polymer is HPMC with a viscosity of 50-300 mPas, such as 100-200 mPas or 130-170 mPas.
41. The unit dosage form according to any one of items 34 to 40, wherein the film-forming polymer is HPMC with a viscosity of 150 mPas.
42. The unit dosage form according to item 34, wherein the film-forming polymer is HPMC with the trade name Affinisol HPMC HME 15 LV.
43. The unit dosage form according to item 34, wherein the film-forming polymer is HPMC with the trade name Affinisol HPMC HME 100 LV.
44. The unit dosage form according to any one of items 34 to 37, 40 or 41, wherein the film forming-polymer is not a HPMC has a substitution type of about 16 to 20% for methoxy and about 26 to 30% for hydroxypropoxy, such as not about 18% for methoxy and not about 28% for hydroxypropoxy, such as not HPMC 1828.
45. The unit dosage form according to item 33, wherein the film-forming polymer is HPC (hydroxypropyl cellulose).
46. The unit dosage form according to item 33, wherein the film-forming polymer is not a hypromellose acetate succinate.
47. The unit dosage form according to item 45, wherein the HPC is a product with the trade name Klucel EF or Klucel ELF.
48. The unit dosage form according to item 33, wherein the film-forming polymer is selected from acrylates, acrylic polymers and co-polymers thereof; polyacrylic acids, polymethacrylates and co-polymers thereof, and polyvinyl alcohol-polyethylene glycol graft-copolymers.
49. The unit dosage form according to item 33, wherein the film-forming polymer is a methacrylic acid-methyl acrylate copolymer.
50. The unit dosage form according to item 33, wherein the film-forming polymer is a methacrylic acid-methyl acrylate copolymers with the trade name Eudragit.
51. The unit dosage form according to item 50, wherein the film-forming polymer is selected from Eudragit E 100, Eudragit RS 100, Eudragit RL 100 and Eudragit RL PO.
52. The unit dosage form according to item 48, wherein the film-forming polymer is a polymer consisting essentially of 75% polyvinyl alcohol units and 25% polyethylene glycol units.
53. The unit dosage form according to item 48, wherein the film-forming polymer is Kollicoat, such as Kollicoat IR.
54. The unit dosage form according to item 33, wherein the film-forming polymer is a polyvinyl caprolactam-polyvinyl acetate-polyethylene glycol graft copolymer.
55. The unit dosage form according to item 33, wherein the film-forming polymer is a product with the trade name Soluplus.
56. The unit dosage form according to item 33, wherein the film-forming polymer is gum selected from the group consisting of acacia gum, guar gum, tragacanth gum, xanthan gum and diutan gum.
57. The unit dosage form according to item 33, wherein the film-forming polymer is alginate selected from the group consisting of sodium alginate, potassium alginate, ammonium alginate, calcium alginate, propylene glycol alginate, alginic acid and mixtures thereof.
58. The unit dosage form according to item 57, wherein the alginate is sodium alginate, potassium alginate or ammonium alginate, or a mixture thereof.
59. The unit dosage form according to any one of items 57 or 58, wherein one or more of the alginate salts comprises from 25 to 35 wt % by weight of α-D-mannuronate and/or from 65 to 75 wt % by weight of α-L-guluronate, and a mean molecular weight of from 30,000 g/mol to 90,000 g/mol.
60. The unit dosage form according to any one of the preceding items, wherein the unit dosage form comprises at least 35 wt % film-forming polymer, such as at least 45 wt %, such as at least 50 wt %, such as at least 55 wt %, such as at least 60 wt %, such as at least 65 wt % film-forming polymer.
61. The unit dosage form according to any one of the preceding items, wherein the unit dosage form comprises no more than 80 wt % film-forming polymer, such as no more than 70 wt % such as no more than 65 wt %, such as no more than 60 wt %, such as no more than 55 wt %, such as no more than 50 wt %, such as no more than 45 wt % film-forming polymer.
62. The unit dosage form according to any one of the preceding items, wherein the unit dosage form comprises or consists of 35 to 80 wt % film-forming polymer, such as 45 to 80 wt %, such as 50 to 65 wt %, such as 55 to 60 wt % film-forming polymer.
63. The unit dosage form according to any one of the preceding items, wherein the unit dosage form comprises or consists of 15 to 45 wt % API and 35 to 80 wt % film forming polymer, such as 25 to 40 wt % API and 50 to 75 wt % film-forming polymer.
64. The unit dosage form according to any one of the preceding items, wherein the unit dosage form comprises or consists of at least 20 wt % API and 35 to 80 wt % film-forming polymer.
65. The unit dosage form according to any one of the preceding items, wherein the unit dosage form is in comprises or consists of at least 20 wt % API and 60 to 80 wt % film-forming polymer.
66. The unit dosage form according to any one of the preceding items, wherein the unit dosage form comprises or consists of 30 to 40 wt % API and 60 to 80 wt % film-forming polymer.
67. The unit dosage form according to any one of the preceding items, wherein the unit dosage form comprises or consists essentially of about 33 wt % API thereof and about 67 wt % film-forming polymer.
68. The unit dosage form according to any one of the preceding items, wherein the unit dosage form comprises or consists of at least 20 wt % midazolam or a pharmaceutically acceptable salt thereof and 35 to 80 wt % film-forming polymer.
69. The unit dosage form according to any one of the preceding items, wherein the unit dosage form comprises or consists of at least 20 wt % midazolam or a pharmaceutically acceptable salt thereof and 60 to 80 wt % film-forming polymer.
70. The unit dosage form according to any one of the preceding items, wherein the unit dosage form comprises or consists of 30 to 40 wt % midazolam or a pharmaceutically acceptable salt thereof and 60 to 80 wt % film-forming polymer.
71. The unit dosage form according to any one of the preceding items, wherein the unit dosage form comprises or consists essentially of about 33 wt % midazolam or a pharmaceutically acceptable salt thereof and about 67 wt % film-forming polymer.
72. The unit dosage form according to any one of the preceding items, wherein the unit dosage form comprises or consists essentially of about 33 wt % midazolam hydrochloride and about 67 wt % film-forming polymer.
73. The unit dosage form according to any one of the preceding items, wherein the unit dosage form further comprises one or more plasticizers.
74. The unit dosage form according to item 73, wherein the one or more plasticizers are selected from a group consisting of glycerol; glycerol monoacetate; citric acid and esters thereof such as triethyl citrate (TEC); diethylene glycol; ethylene glycol; fatty acid esters; PEG, such as PEG 400, PEG 600 or PEG 4000; polyethylene-propylene glycols; propylene glycol; phthalic acid; polyalkylene oxides; sorbitol, triacetin and xylitol.
75. The unit dosage form according to any one of items 73 or 74, wherein the plasticizer is glycerol.
76. The unit dosage form according to any one of items 73 or 74, wherein the plasticizer is TEC.
77. The unit dosage form according to any one items 73 or 74, wherein the plasticizer is poloxamer 407.
78. The unit dosage form according to any one of items 73 or 74, wherein the plasticizer is Kollicoat IR.
79. The unit dosage form according to any one of the preceding items, wherein the unit dosage form comprises at least 3 wt % plasticizer, such as at least 5 wt %, such as at least 10 wt %, such as at least 30 wt % plasticizer.
80. The unit dosage form according to any one of the preceding items, wherein the unit dosage form comprises no more than 30 wt % plasticizer, such as no more than 20 wt %, such as no more than 15 wt %, such as no more than 10 wt %, such as no more than 5 wt % plasticizer.
81. The unit dosage form according to any one of the preceding items, wherein the unit dosage form comprises 3 to 35 wt % plasticizer, such as 4 to 10 wt %, such as about 5 wt % plasticizer.
82. The unit dosage form according to any one of items 1 to 78, wherein the unit dosage form comprises more than 3 wt % but less than 5 wt % plasticizer.
83. The unit dosage form according to any one of items 1 to 78, wherein the unit dosage form comprises about 3 wt % plasticizer.
84. The unit dosage form according to any one of the preceding items, wherein the unit dosage form comprises a combination of two plasticizers.
85. The unit dosage form according to any one of the preceding items, wherein the unit dosage form comprises a combination of two plasticizers selected from group consisting of glycerol, TEC, poloxamer 407 and Kollicoat IR, such as glycerol and TEC, or glycerol and poloxamer 407, or glycerol and Kollicoat IR, or TEC and poloxamer 407, or TEC and Kollicoat IR, or poloxamer 407 and Kollicoat IR.
86. The unit dosage form according to any one of items 1 to 78, wherein the unit dosage form comprises 15 to 45 wt % API, 35 to 80 wt % film-forming polymer and 3 to 35 wt % plasticizer.
87. The unit dosage form according to any one of items 1 to 75, wherein the unit dosage form comprises 15 to 45 wt % midazolam, or a pharmaceutically acceptable salt thereof; 35 to 80 wt % HPMC and 3 to 15 wt % glycerol.
88. The unit dosage form according to any one of items 1 to 72, wherein the unit dosage form does not comprise any plasticizer.
89. The unit dosage form according to any one of items 1 to 72, wherein the film-forming polymer is HPMC 2528 and the unit dosage form does not comprise any plasticizer.
90. The unit dosage form according to any one of items 1 to 31, wherein the film-forming polymer is HPMC with the trade name Affinisol HPMC HME 15 LV and the unit dosage form does not comprise any plasticizer.
91. The unit dosage form according to any one of items 1 to 31, wherein the film-forming polymer is HPMC with the trade name Affinisol HPMC HME 100 LV and the unit dosage form does not comprise any plasticizer.
92. The unit dosage form according to any one of items 1 to 30, wherein the film-forming polymer is HPC and the unit dosage form does not comprise any plasticizer.
93. The unit dosage form according to any one of the preceding items, wherein the unit dosage form comprises one or more additives selected from a group consisting of colorants, pigments, taste masking agents and flavouring agents.
94. The unit dosage form according to any one of the preceding items, wherein the unit dosage form consists of an API, one or more film-forming polymers, one or more flavouring agents and a pigment.
95. The unit dosage form according to any one of the preceding items, wherein the unit dosage form consists of an API, one or more film-forming polymers and a pigment.
96. The unit dosage form according to any one of items 1 to 93, wherein the unit dosage form further comprises a colorant.
97. The unit dosage form according to any one of items 93 or 96, wherein the colorant is a pigment.
98. The unit dosage form according to any one of items 93 to 97, wherein the pigment is selected from a group consisting of yellow iron oxide, red iron oxide and black iron oxide.
99. The unit dosage form according to any one of the preceding items, wherein the unit dosage form comprises at least 0.2 wt % pigment, such as at least 0.5 wt %, such as at least 1 wt % pigment.
100. The unit dosage form according to any one of the preceding items, wherein the unit dosage form comprises no more than 10 wt % pigment, such as no more than 5 wt %, such as no more than 2 wt %, such as no more than 1 wt % pigment.
101. The unit dosage form according to any one of the preceding items, wherein the unit dosage form comprises 0.5 to 5 wt % pigment, such as about 1 wt % pigment.
102. The unit dosage form according to any previous item, wherein the unit dosage form comprises a flavouring agent that mitigates the bitter sensation emanating from the API.
103. The unit dosage form according to any one of the preceding items, wherein the unit dosage form comprises at least 1 wt % flavouring agent, such as at least 2 wt %, such as at least 5 wt % flavouring agent.
104. The unit dosage form according to any one of the preceding items, wherein the unit dosage form comprises no more than 20 wt % flavouring agent, such as no more than 10 wt % flavouring agent.
105. The unit dosage form according to any one of the preceding items with the proviso that the sum of the wt % of the components does not exceed 100 wt %.
106. The unit dosage form according to any one of the preceding items, wherein the unit dosage form is 50 to 150 μm thick, such as 60 to 120 μm thick, such as 70 to 110 μm thick, such as 80 to 100 μm thick.
107. The unit dosage form according to any one of the preceding items, wherein the unit dosage form is 40 to 100 μm thick, such as 50 to 90 μm thick, such as 60 to 80 μm thick.
108. The unit dosage form according to any one of the preceding items, wherein the unit dosage form has a dimension of X×Y×Z, wherein X is in the range of 0.5 to 5 cm; Y is in the range of 0.5 to 5 cm; and Z is in the range of 15 to 150 μm.
109. The unit dosage form according to any one of the preceding items, wherein the unit dosage form has a dimension of X×Y×Z, wherein X is at least 0.5 cm, such as at least 1 cm, such as at least 1.5 cm, such as at least 2 cm.
110. The unit dosage form according to any one of the preceding items, wherein the unit dosage form has a dimension of X×Y×Z, wherein X is no more than 5 cm, such as no more than 4.5 cm, such as no more than 4 cm, such as no more than 3.5 cm, such as no more than 3 cm.
111. The unit dosage form according to any one of the preceding items, wherein the unit dosage form has a dimension of X×Y×Z, wherein X is in the range of 0.5 to 5 cm, such as in the range of 1 to 3 cm, for example in the range of 1 to 2 cm.
112. The unit dosage form according to any one of the preceding items, wherein the unit dosage form has a dimension of X×Y×Z, wherein Y is at least 0.5 cm, such as at least 1 cm, such as at least 1.5 cm, such as at least 2 cm.
113. The unit dosage form according to any one of the preceding items, wherein the unit dosage form has a dimension of X×Y×Z, wherein Y is no more than 5 cm, such as no more than 4.5 cm, such as no more than 4 cm, such as no more than 3.5 cm, such as no more than 3 cm.
114. The unit dosage form according to any one of the preceding items, wherein the unit dosage form has a dimension of X×Y×Z, wherein Y is in the range of 0.5 to 5 cm, such as in the range of 1 to 3 cm, for example in the range of 2 to 3 cm.
115. The unit dosage form according to any one of the preceding items, wherein the unit dosage form has a dimension of X×Y×Z, wherein Z is at least 5 μm, such as at least 25 μm, such as at least 50 μm, such as at least 75 μm, such as at least 100 μm.
116. The unit dosage form according to any one of the preceding items, wherein the unit dosage form has a dimension of X×Y×Z, wherein Z is no more than 1 mm, such as no more than 750 μm, such as no more than 500 μm, such as no more than 250 μm, such as no more than 125 μm.
117. The unit dosage form according to any one of the preceding items, wherein the unit dosage form has a dimension of X×Y×Z, wherein Z is in the range of 5 μm to 750 μm, such as in the range of 30 to 150 μm, such as 50 to 120 μm, such as 70 to 110 μm.
118. The unit dosage form according to any one of the preceding items, wherein the unit dosage form has a dimension of X×Y×Z, wherein X is in the range of 0.5 to 5 cm; Y is on the range of 0.5 to 5 cm; and Z is in the range of 30 μm to 150 μm.
119. The unit dosage form according to any one of the preceding items, wherein the unit dosage form has a dimension of X×Y×Z, wherein X is in the range of 1 to 3 cm; Y is on the range of 1 to 3 cm; and Z is in the range of 50 μm to 150 μm.
120. The unit dosage form according to any one of the preceding items, wherein the unit dosage form is rectangular and has dimensions of about 1.5 cm×2.5 cm×90 μm.
121. The unit dosage form according to any one of the preceding items, wherein the unit dosage form is rectangular and has an area X×Y of 1 to 6 cm², such as 1.5 to 5 cm², such as 3 to 4.5 cm², such as 3.5 to 4 cm².
122. The unit dosage form according to any one of the preceding items, wherein the unit dosage form has a coat weight of 50 to 150 g/m², such as 75 to 125 g/m², such as 80 to 110 g/m², such as about 90 g/m².
123. The unit dosage form according to any one of the preceding items, wherein the unit dosage form has a rectangular form and an area of 1 to 6 cm², such as 1.5 to 5 cm², such as 3 to 4.5 cm², such as 3.5 to 4 cm², and is 30 to 150 μm thick, such as 50 to 120 μm thick, such as 70 to 110 μm thick.
124. The unit dosage form according to any one of the preceding items, wherein the unit dosage form has a rectangular form and the area. X×Y is 1 to 6 cm², such as 1.5 to 5 cm², such as 3 to 4.5 cm², such as 3.5 to 4 cm², and a coat weight of 50 to 150 g/m², such as 75 to 125 g/m², such as 80 to 110 g/m², such as about 90 g/m².
125. The unit dosage according to any one of the preceding items, wherein the unit dosage form is oval and has an area of 2 to 5 cm², such as 2.5 to 4.5 cm².
126. The unit dosage form according to any one of the preceding items, wherein the unit dosage form is circular and has an area of 2 to 5 cm², such as 2.5 to 4.5 cm².
127. The unit dosage form according to any one of the preceding items, wherein the unit dosage form is circular and has a radius of 8 mm to 13 mm.
128. The unit dosage form according to any one of the preceding items, wherein the unit dosage form comprises 10 mg midazolam and has dimensions of about 1.5 cm×2.5 cm×70 to 110 μm.
129. The unit dosage form according to any one of the preceding items, wherein the unit dosage form consists of a single layer.
130. The unit dosage form according to any one of the preceding items, wherein the film has a moderately high dissolution rate.
131. The unit dosage form according to any one of the preceding items, wherein the film has moderately high in vitro dissolution rate.
132. The unit dosage form according to any one of the preceding items, wherein at least 40% of the API has been dissolved within 10 minutes in the USP Dissolution Apparatus 2—Paddle, such as at least 50%, such as at least 60%, such as at least 70%, such as at least 80%, such as at least 90% of the API has been dissolved within 10 minutes.
133. The unit dosage form according to any one of the preceding items, wherein at least 85% of the API has been dissolved within 10 minutes in the USP Dissolution Apparatus 2—Paddle, such as at least 90%, such as at least 95% of the API has been dissolved within 10 minutes.
134. The unit dosage form according to any one of the preceding items, wherein at least 90% of the API has been dissolved within 10 minutes in the USP Dissolution Apparatus 2—Paddle, such as at least 95%, such as at least 97.5%, such as 100% of the API has been dissolved within 10 minutes.
135. The unit dosage form according to any one of the preceding items, wherein no more than 97.5% of the API has been dissolved within 10 minutes in the USP Dissolution Apparatus 2—Paddle, such as no more than 95%, such as no more than 90%, such as no more than 85%, such as no more than 80%, such as no more than 75%, such as no more than 70%, such as no more than 60% such as no more than 50% of the API has been dissolved within 10 minutes.
136. The unit dosage form according to any one of the preceding items, wherein at least 30% of the API has been dissolved within 5 minutes in the USP Dissolution Apparatus 2—Paddle, such as at least 40%, such as at least 50%, such as at least 60%, such as at least 70%, such as at least 80%, such as at least 90% of the API has been dissolved within 5 minutes.
137. The unit dosage form according to any one of the preceding items, wherein at least 85% of the API has been dissolved within 5 minutes in the USP Dissolution Apparatus 2—Paddle, such as at least 90%, such as at least 95% of the API has been dissolved within 5 minutes.
138. The unit dosage form according to any one of the preceding items, wherein no more than 97.5% of the API has been dissolved within 5 minutes in the USP Dissolution Apparatus 2—Paddle, such as no more than 95%, such as no more than 90% of the API has been dissolved within 5 minutes.
139. The unit dosage form according to any one of the preceding items, wherein no more than 97.5% of the API has been dissolved within 5 minutes in the USP Dissolution Apparatus 2—Paddle, such as no more than 90%, such as no more than 85%, such as no more than 80%, such as no more than 75%, such as no more than 70%, such as no more than 65%, such as no more than 60%, such as no more than 55%, such as no more than 50%, such as no more than 45%, such as no more than 40% of the API has been dissolved within 5 minutes.
140. The unit dosage form according to any one of the preceding items, wherein at least 20% of the API has been dissolved within 1 minute in the USP Dissolution Apparatus 2—Paddle, such as at least than 30%, such as at least 40%, such as at least 50%, such as at least 60%, such as at least 70% of the API has been dissolved within 1 minute.
141. The unit dosage form according to any one of the preceding items, wherein no more than 90% of the API has been dissolved within 1 minute in the USP Dissolution Apparatus 2—Paddle, such as no more than 80%, such as no more than 75%, such as no more than 70%, such as no more than 65%, such as no more than 60%, such as no more than 55%, such as no more than 50%, such as no more than 45%, such as no more than 40% of the API has been dissolved within 1 minute.
142. The unit dosage form according to any one of the preceding items, wherein no more than 90% of the API, has been dissolved within 1 minute in the USP Dissolution Apparatus 2—Paddle.
143. The unit dosage form according to any one of the preceding items, wherein at least 90% of the API has been dissolved within 10 minutes but no more than 97.5% has been dissolved within 5 minutes in the USP Dissolution Apparatus 2—Paddle.
144. The unit dosage form according to any one of items 132 to 143, wherein the dissolution medium of the USP Dissolution Apparatus 2—Paddle is phosphate buffer pH 6.8.
145. The unit dosage form according to any one of items 132 to 143, wherein USP Dissolution Apparatus 2—Paddle is conducted at 37° C.±0.5° C., 75 rpm in 1000 mL phosphate buffer pH 6.8.
146. The unit dosage form according to any one of items 132 to 143, wherein the dissolution medium of the USP Dissolution Apparatus 2—Paddle is phosphate buffer pH 6.8 with 0.08% Tween20.
147. The unit dosage form according to any one of items 132 to 143, wherein USP Dissolution Apparatus 2—Paddle is conducted at 37° C.±0.5° C., 75 rpm in 1000 mL phosphate buffer pH 6.8 with 0.08% Tween20.
148. The unit dosage form according to any one of items 132 to 143, wherein the dissolution medium of the USP Dissolution Apparatus 2—Paddle is water.
149. The unit dosage form according to any one of items 132 to 143, wherein USP Dissolution Apparatus 2—Paddle is conducted at 37° C.±0.5° C., 75 rpm in 1000 mL water.
150. The unit dosage form according to any one of items 1 to 129, wherein at least 5% but not more than 55% of the API has been dissolved within 5 minutes, and at least 30% but no more than 70% has been dissolved within 10 minutes, and less than 90% has been dissolved within 20 minutes, when USP Dissolution Apparatus 2—Paddle is conducted at 37° C.±0.5° C., 75 rpm in 1000 mL phosphate buffer pH 6.8 with or without 0.08% Tween20.
151. The unit dosage form according to any one of the preceding items, wherein the unit dosage form is a mucoadhesive film.
152. The unit dosage form according to any one of the preceding items, wherein the unit dosage form is for buccal administration.
153. The unit dosage form according to any one of items 1 to 151, wherein the unit dosage form is for oral administration.
154. The unit dosage form according to any one of the preceding items for use as a medicament.
155. The unit dosage form according to any one of the preceding items for use in the acute treatment of seizures in a subject.
156. The unit dosage form for use according to item 155, wherein the seizures are caused by epilepsy or another disease or condition that may cause seizures.
157. The unit dosage form for use according to any one of items 155 or 156, wherein the subject is suffering from epilepsy or another disease or condition that may cause seizures.
158. The unit dosage form for use according to item 157, wherein the epilepsy is generalised epilepsy or partial epilepsy.
159. The unit dosage form for use according to item 157, wherein the disease or condition that may cause seizures is selected from the group consisting of fever caused by malaria, fever of other causes, poisoning, tetanus, brain tumours, Lennox-Gastaut syndrome, tuberous sclerosis complex and Dravet syndrome.
160. The unit dosage form for use according to any one of items 155 to 159 wherein the seizures are selected from the group consisting of cluster seizures, seizure convulsions, convulsions, spasms, prolonged acute convulsive seizures, stereotypic episodes of frequent seizure activity that are distinct from a patient's usual seizure pattern, status epilepticus and convulsive refractory status epilepticus.
161. The unit dosage form for use according to any one of items 155 to 160 wherein the seizures are ongoing, acute seizures.
162. The unit dosage form for use according to any one of items 155 to 161, wherein the unit dosage form is an oral film for buccal administration.
163. The unit dosage form according to any one of items 1 to 153 for use in the induction of moderate sedation or pre-sedation in a subject.
164. The unit dosage form according to any one of items 1 to 153 for use in moderate sedation before diagnostic, therapeutic or surgical procedures or pre-sedation before anaesthesia.
165. The unit dosage form for use according to item 164, wherein the diagnostic, therapeutic or surgical procedures are within odontology.
166. The unit dosage form for use according to any one of items 154 to 165, wherein the unit dosage form is to be applied onto the tongue.
167. The unit dosage form for use according to any one of items 154 to 165, wherein the unit dosage form is to be applied sublingually.
168. The unit dosage form for use according to any one of items 154 to 165, wherein the unit dosage form is to be applied buccally.
169. The unit dosage form according to any one of items 1 to 153 for use in a method of acute treatment of seizures and/or for induction of moderate sedation or pre-sedation in a subject, wherein the unit dosage form is for oral administration.
170. The unit dosage form according to any one of items 1 to 153 for use in a method of acute treatment of seizures and/or for induction of moderate sedation or pre-sedation in a subject, wherein the unit dosage form is for buccal administration.
171. The unit dosage form according to any one of items 1 to 153 for use in a method of acute treatment of seizures and/or for induction of moderate sedation or pre-sedation in a subject, wherein the unit dosage form is for sublingual administration.
172. The unit dosage form for use according to any one of items 154 to 171, wherein the unit dosage form provides a plasma drug concentration-time profile of midazolam where the mean Cₘₐₓ is about 80% to about 125%, such as 80.00% to 125.00%, of 64.32 ng/ml after administration with a single unit dosage form comprising 10 mg midazolam (defined as base) applied on the buccal mucosa.
173. The unit dosage form for use according to any one of items 154 to 171, wherein the unit dosage form provides a plasma drug concentration-time profile of midazolam where the mean Cₘₐₓ is 50 to 85 ng/ml, such as 60 to 70 ng/ml, such as 63 to 66 ng/ml, such as about 64 ng/ml after administration with a single unit dosage form comprising 10 mg midazolam (defined as base) applied on the buccal mucosa.
174. The dosage form for use according to any one of items 172 or 173, wherein the Cₘₐₓ values referred to is those of adult male healthy volunteers.
175. The unit dosage form for use according to any one of items 154 to 171, wherein the film provides a plasma drug concentration-time profile of midazolam where the mean AUC₀₋ₜ is about 80% to about 125%, such as 80.00% to 125.00%, of 223.65 ng*h/mL after administration with a single unit dosage form comprising 10 mg midazolam (defined as base) applied on the buccal mucosa.
176. The unit dosage form for use according to any one of items 154 to 171, wherein the film provides a plasma drug concentration-time profile of midazolam where the mean AUC₀₋ₜ is 175 to 280 ng*h/mL, such as 200 to 250 ng*h/mL, such as 220 to 225 ng*h/mL, such as about 224 ng*h/mL after administration with a single unit dosage form comprising 10 mg midazolam (defined as base) applied on the buccal mucosa.
177. The dosage form for use according to any one of items 175 or 176, wherein the AUC₀₋ₜ values referred to is those of adult male healthy volunteers.
178. The unit dosage form for use according to any one of items 154 to 171, wherein the unit dosage form provides a plasma drug concentration-time profile of midazolam where the mean Cₘₐₓ is about 80% to about 125%, such as 80.00% to 125.00%, of 64.32 ng/mL after administration with a single unit dosage form containing a dose corresponding to 0.13 mg midazolam (base defined as the base) per kg body weight of the subject, applied on the buccal mucosa.
179. The unit dosage form for use according to any one of items 154 to 171, wherein the unit dosage form provides a plasma drug concentration-time profile of midazolam where the mean Cₘₐₓ is 50 to 85 ng/ml, such as 60 to 70 ng/ml, such as 63 to 66 ng/ml, such as about 64 ng/ml after administration with a single unit dosage form containing a dose corresponding to 0.13 mg midazolam (base defined as the base) per kg body weight of the subject, applied on the buccal mucosa.
180. The unit dosage form for use according to any one of items 154 to 171, wherein the film provides a plasma drug concentration-time profile of midazolam where the mean AUC₀₋ₜ is about 80% to about 125%, such as 80.00% to 125.00%, of 223.65 ng*h/mL after administration with a single unit dosage form containing a dose corresponding to 0.13 mg midazolam (defined as the base) per kg body weight of the subject, applied on the buccal mucosa.
181. The unit dosage form for use according to any one of items 154 to 171, wherein the film provides a plasma drug concentration-time profile of midazolam where the mean AUC₀₋ₜ is 175 to 280 ng*h/mL, such as 200 to 250 ng*h/mL, such as 220 to 225 ng*h/mL, such as about 224 ng*h/mL after administration with a single unit dosage form containing a dose corresponding to 0.13 mg midazolam (defined as the base) per kg body weight of the subject, applied on the buccal mucosa.
182. The unit dosage form for use according to any one of items 154 to 171, wherein the unit dosage form provides a plasma drug concentration-time profile of midazolam where the mean Cₘₐₓ is about 80% to about 125%, such as 80.00% to 125.00%, of 68.72 ng/ml after administration with a single unit dosage form comprising 10 mg midazolam (defined as base) applied onto the tongue of the subject.
183. The unit dosage form for use according to any one of items 154 to 171, wherein the unit dosage form provides a plasma drug concentration-time profile of midazolam where the mean Cₘₐₓ is 50 to 90 ng/ml, such as 60 to 75 ng/ml, such as 65 to 70 ng/ml, such as about 69 ng/ml after administration with a single unit dosage form comprising 10 mg midazolam (defined as base) applied onto the tongue of the subject.
184. The dosage form for use according to any one of items 182 or 183, wherein the Cₘₐₓ values referred to is those of adult male healthy volunteers.
185. The unit dosage form for use according to any one of items 154 to 171, wherein the film provides a plasma drug concentration-time profile of midazolam where the mean AUC₀₋ₜ is about 80% to about 125%, such as 80.00% to 125.00%, of 218.97 ng*h/mL after administration with a single unit dosage form comprising 10 mg midazolam (defined as base) applied onto the tongue of the subject.
186. The unit dosage form for use according to any one of items 154 to 171, wherein the film provides a plasma drug concentration-time profile of midazolam where the mean AUC₀₋ₜ is 175 to 280 ng*h/mL, such as 200 to 250 ng*h/mL, such as 215 to 225 ng*h/mL, such as about 219 ng*h/mL after administration with a single unit dosage form comprising 10 mg midazolam (defined as base) applied onto the tongue of the subject.
187. The dosage form for use according to any one of items 185 or 186, wherein the AUC₀₋ₜ values referred to is those of adult male healthy volunteers.
188. The unit dosage form for use according to any one of items 154 to 171, wherein the unit dosage form provides a plasma drug concentration-time profile of midazolam where the mean Cₘₐₓ is about 80% to about 125%, such as 80.00% to 125.00%, of 68.72 ng/ml after administration with a single unit dosage form containing a dose corresponding to 0.13 mg midazolam (base defined as the base) per kg body weight of the subject, applied onto the tongue of the subject.
189. The unit dosage form for use according to any one of items 154 to 171, wherein the unit dosage form provides a plasma drug concentration-time profile of midazolam where the mean Cₘₐₓ is 50 to 90 ng/ml, such as 60 to 75 ng/ml, such as 65 to 70 ng/ml, such as about 69 ng/ml after administration with a single unit dosage form containing a dose corresponding to 0.13 mg midazolam (base defined as the base) per kg body weight of the subject, applied onto the tongue of the subject.
190. The unit dosage form for use according to any one of items 154 to 171, wherein the film provides a plasma drug concentration-time profile of midazolam where the mean AUC₀₋ₜ is about 80% to about 125%, such as 80.00% to 125.00%, of 218.97 ng*h/mL after administration with a single unit dosage form containing a dose corresponding to 0.13 mg midazolam (defined as the base) per kg body weight of the subject, applied onto the tongue of the subject.
191. The unit dosage form for use according to any one of items 154 to 171, wherein the film provides a plasma drug concentration-time profile of midazolam where the mean AUC₀₋ₜ is 175 to 280 ng*h/mL, such as 200 to 250 ng*h/mL, such as 215 to 225 ng*h/mL, such as about 219 ng*h/mL after administration with a single unit dosage form containing a dose corresponding to 0.13 mg midazolam (defined as the base) per kg body weight of the subject, applied on the tongue of the subject.
192. The unit dosage form for use according to any one of items 154 to 191, wherein the resulting plasma curve over time for a subject, after oral administration of the unit dosage form, i.e., onto the tongue, does not show a double-peak phenomena.
193. The unit dosage form for use according to any one of items 154 to 192, wherein the subject is a mammal.
194. The unit dosage form for use according to item 193, wherein the mammal is a human.
195. The unit dosage form for use according to items 193, wherein the mammal is a dog, horse or a cat.
196. A method for treating seizures in a subject, comprising administering the unit dosage according to any one of items 1 to 153 to the subject in need thereof.
197. A method for induction of moderate sedation or pre-sedation in a subject, comprising administering the unit dosage according to any one of items 1 to 153 to the subject.
198. Use of the unit dosage form according to any one of items 1 to 153 in the manufacture of a medicament for the treatment of seizures.
199. Use of the unit dosage form according to any one of items 1 to 153 in the manufacture of a medicament for induction of moderate sedation or pre-sedation in a subject.
200. A process for producing a unit dosage according to any one of items 1 to 153, wherein the process comprises the sequential steps of:
   a) mixing midazolam or a pharmaceutically acceptable salt thereof and one or more film-forming polymers in a process solvent to provide a wet mix;
   b) casting the wet mix obtained in step a) to provide a wet film;
   c) drying the wet film of b) to obtain a dry film; and
   d) cutting the dry film of c) into a unit dosage form.
201. A process for producing an oral film comprising an active pharmaceutical ingredient (API) and one or more film-forming polymers, wherein the process comprises the sequential steps of:
   i. mixing the API and one or more film-forming polymers in one or more solvents to provide a wet mix;
   ii. casting the wet mix obtained in step a) to provide a wet film; and
   iii. drying said wet film to obtain the dry oral film.
202. A process for producing a unit dosage form in the form of an oral film comprising an active pharmaceutical ingredient (API) and one or more film-forming polymers, wherein the process comprises the sequential steps of:
   i. mixing the API and one or more film-forming polymers in one or more solvents to provide a wet mix;
   ii. casting the wet mix obtained in step a) to provide a wet film;
   iii. drying said wet film to obtain a dry film; and
   iv. cutting the dry film of step c) into a unit dosage form.
203. The process according to any one of items 200 to 202, wherein the API is added before the one or more film-forming polymers are added, and a homogeneous mix, with the API in suspended state, is achieved before those polymers are added.
204. The process according to any one of items 200 to 203, wherein step a) comprises the sequential steps of:
   i. mixing the API with the solvent to obtain a homogenous suspension;
   ii. optionally adding a plasticizer and/or a pigment to the suspension in i) and mixing to obtain a homogenous mix; and
   iii. adding the film-forming polymer to the mix of ii) and mixing to obtain a wet mix in which the film-forming polymer is dissolved but the API is not dissolved.
205. The process according to any one of items 200 to 202, wherein the one or more film-forming polymers is dissolved in the solvent before the API is added.
206. The process according to any one of items 200 to 202 or 205, wherein step a) comprises the sequential steps of:
   i. mixing the film-forming polymer in process solvent to obtain a homogenous solution;
   ii. adding the API and optionally a plasticizer and/or a pigment to the solution in a) and mixing to obtain a wet mix in which the film-forming polymer is dissolved but the API is not dissolved.
207. The process according to any one of items 200 to 206, wherein the process solvent does not dissolve the API.
208. The process according to any one of items 200 to 207, wherein the process solvent is a solvent wherein no more than 10 wt %, such as no more than 5 wt %, such as no more than 2.5 wt % of the API is dissolved after stirring a mixture of API/solvent in a 5:95 weight ratio for 2 hours at room temperature.
209. The process according to any one of items 200 to 208, wherein the process solvent dissolves the film-forming polymer.
210. The process according to any one of items 200 to 209, wherein the process solvent is a solvent wherein no more than 10 wt %, such as no more than 5 wt %, such as no more than 2.5 wt %, of the added film-forming polymer is non-dissolved after stirring a mixture of film-forming polymer/solvent in a 15:85 weight ratio for 2 hours at room temperature.
211. The process according to any one of items 200 to 210, wherein the process solvent is a non-aqueous solvent.
212. The process according to any one of items 200 to 210, wherein the process solvent is essentially free from water and comprises or consists of one or more non-aqueous solvents.
213. The process according to any one of items 200 to 206, wherein the process solvent comprises or consists of one or more solvent selected from the group consisting of acetone, 1-butanol, 2-butanol, butyl acetate, ethyl acetate, isobutyl acetate, isopropyl acetate, methyl acetate, methyl ethyl ketone, 2-methyl-1-propanol, 1-pentanol, 1-propanol, 2-propanol, propyl acetate and triethylamine.
214. The process according to any one of items 200 to 206, wherein the process solvent is selected from the group consisting of acetone, ethyl acetate, isobutyl acetate, methyl acetate, 1-pentanol and 2-propanol.
215. The process according to any one of items 200 to 206, wherein the process solvent is selected from the group consisting of acetone, ethyl acetate, isobutyl acetate and methyl acetate.
216. The process according to any one of items 200 to 206, wherein the process solvent is ethyl acetate.
217. The process according to any one of items 200 to 210, wherein the solvent comprises or consists of water and one or more non-aqueous solvents.
218. The process according to any one of items 200 to 217, wherein at least 90 wt % of the total amount of the film-forming polymer is dissolved in the wet mix, such as at least 95 wt %, such as at least 98 wt %, such as 100 wt %.
219. The process according to any one of items 200 to 218, wherein not more than 20 wt % of the total amount of API is dissolved in the wet mix, such as not more than 10 wt %, such as not more than wt 5%, such as not more than 2 wt %.
220. The process according to any one of items 200 to 219, wherein at least 80 wt % of the total amount of the API remains suspended throughout the process, such as at least 90 wt %, such as at least 95 wt %, such as at least 98 wt %.
221. The process according to any one of items 200 to 220, wherein the film-forming polymer(s) but not the API dissolves in the wet mix in step a), thereby achieving a dry film in step c) in which the API is suspended as solid particles inside the formed film.
222. The process according to any one of items 200 to 221, wherein the suspended API has the same particle size and crystalline type as the API that was added in step a).
223. The process according to any one of items 200 to 222, wherein the dry film is a monolayered film.
224. The process according to any one of items 200 to 223, wherein the film consists of the API and the film-forming polymer, and optionally a colorant and/or a flavouring agent
225. The process according to any one of items 200 to 224, wherein the API is midazolam or a pharmaceutically acceptable salt thereof.
226. The process according to any one of items 200 to 225, wherein the API is midazolam hydrochloride.
227. The process according to any one of items 200 to 225, wherein the API is or midazolam maleate.
228. The process according to any one of items 200 to 225, wherein the API is added in step a) as midazolam hydrochloride.
229. The process according to any one of items 200 to 228, wherein the film-forming polymer is selected from the group consisting of:
   i. HPMC 2528;
   ii. HPC;
   iii. hypromellose acetate succinate;
   iv. methacrylic acid-methyl acrylate copolymers; and
   v. polyvinyl caprolactam-polyvinyl acetate-polyethylene glycol graft copolymer.
230. The process according to item 229, wherein the process solvent is ethyl acetate.
231. The process according to any one of items 200 to 229, wherein the solvent is ethyl acetate and the API is midazolam hydrochloride or midazolam maleate.
232. The process according to any one of items 200 to 231, wherein the wet film thickness is 300 to 800 μm, such as 400 to 700 μm, such as 550 to 650 μm.
233. The process according to any one of items 200 to 232, wherein the drying is performed at 40 to 120° C., such as 50 to 110° C., such as 60 to 100° C., such as 40 to 100° C., such as 40 to 80° C.
234. The process according to any one of items 200 to 233, wherein the drying is performed by using several temperature zones.
235. The process according to anyone of items 200 to 234, wherein the wet film is dried until the residual process solvent content is no more than 5 wt %, such as no more than 4 wt %, such as no more than 3 wt % such as no more than 2 wt %, such as no more than 1 wt %.
236. The process according to any one of items 200 to 235, wherein the amount of residual process solvent in the dry film is not less than 1 000 ppm (parts per million), such as not less than 5 000 ppm, such that not less than 10 000 ppm, such as less not than 25 000 ppm.
237. The process according to any one of items 200 to 236, wherein the solvent is ethyl acetate, the API is midazolam hydrochloride or midazolam maleate, and the amount of residual ethyl acetate is not less than 1 000 ppm (parts per million), such as not less than 5 000 ppm, such that not less than 10 000 ppm, such as less not than 25 000 ppm.
238. The process according to any one of items 200 to 237, wherein the film-forming polymer is HPMC2528.
239. The process according to any one of items 200 to 237, wherein the film-forming polymer is HPC.
240. An oral film obtained by the process of any one of items 201 or 203 to 239.
241. A unit dosage form obtained by the process of any one of items 202 to 239.

### EXAMPLES

### Materials

Components used in the Examples below, and their intended or hypothesized functions, is presented in this table:

| | Intended | | Supplier (Country) |
|---|---|---|---|
| Excipient/material | function* | Compendial | (Manufacturer, if other) |
| Midazolam | API | PhEur (base) | Cambrex (Italy) |
| hydrochloride | | | |
| (midazolam HCl) | | | |
| Midazolam, base | API | PhEur | Cambrex (Italy) |
| HPMC Metolose | Film-forming | PhEur / USP/ | SEPPIC (France) |
| 60SH-50 | polymer | JPE | (ShinEtsu) |
| HPMC | Film-forming | PhEur / USP/ | SEPPIC (France) |
| Pharmacoat 603 | polymer | JPE | (ShinEtsu) |
| HPMC Benecel E- | Film-forming | USP | Ashland (US) |
| 50 | polymer | | |
| PVA Gohsenol | Film-forming | PhEur / USP/ | HARKE Pharma |
| EG-05PW | polymer | JPE | (Germany) |
| | | | (Nippon Gohsei) |
| PVA Gohsenol | Film-forming | PhEur / USP/ | HARKE Pharma |
| EG-40PW | polymer | JPE | (Germany) |
| | | | (Nippon Gohsei) |
| Pullulan | Film-forming | PhEur | Sigma-Aldrich (Sweden) |
| | polymer | | (*) |
| AQOAT AS-LG | Film-forming | USP /JPE | SEPPIC (France) |
| | polymer | | (Shin-Etsu) |
| Affinisol HPMC | Film-forming | N/A* | Colorcon (France) |
| HME 100LV | polymer | | (DUPONT) |
| Affinisol HPMC | Film-forming | N/A* | Colorcon (France) |
| HME 15LV | polymer | | (DUPONT) |
| Natrosol 250L | Film-forming | PhEur / USP | IMCD (France) |
| | polymer | | (ASHLAND) |
| Natrosol 250HX | Film-forming | PhEur / USP | IMCD (France) |
| | polymer | | (ASHLAND) |
| Eudragit L 100-55 | Film-forming | PhEur / USP- | Evonik (Germany) |
| | polymer | NF | |
| Pemulen TR-1 | Film-forming | USP-NF | Lubrizol (US) |
| | polymer | | |
| Sodium alginate | Film-forming | N/A* | Sigma-Aldrich |
| | polymer | | |
| Kappa-carragenan | Film-forming | N/A* | Sigma-Aldrich |
| | polymer | | |
| Iota-carragenan | Film-forming | N/A* | Sigma-Aldrich |
| | polymer | | |
| Rheocare XGN | Film-forming | N/A* | BASF (Germany) |
| | polymer | | |
| PVP 360 | Film-forming | N/A* | Sigma-Aldrich |
| | polymer | | |
| Eudragit E 100 | Film-forming | PhEur / USP- | Evonik (Germany) |
| | polymer | NF | |
| Eudragit RS 100 | Film-forming | PhEur / USP- | Evonik (Germany) |
| | polymer | NF | |
| Eudragit RL 100 | Film-forming | PhEur /USP- | Evonik (Germany) |
| | polymer | NF | |
| Eudragit RL PO | Film-forming | PhEur / USP- | Evonik (Germany) |
| | polymer | NF | |
| Kollicoat IR | Film-forming | PhEur / USP | BTC (Germany) |
| | polymer, or | | (BASF Pharma) |
| | Plasticizer | | |
| Soluplus | Film-forming | US-DMF | BTC (France) |
| | polymer | #23504 | (BASF) |
| | | US-DMF | |
| | | #23626 | |
| Triethyl citrate | Plasticizer | Reagent | Acros organics (Belgium) |
| (TEC) | | Laboratory | (Fisher Scientific) |
| Poloxamer 407 | Plasticizer | PhEur / USP/ | BTC (France) (BASF) |
| | | JPE | |
| Glycerol 99.7% | Plasticizer | PhEur / USP | Univar (Germany) (Olin)* |
| Polysorbate 80 | Solubilizer and/or | N/A* | * |
| | plasticizer | | |
| Xylitol | Sweetening agent | N/A* | * |
| | and/or plasticizer | | |
| Sorbitol | Sweetening agent | N/A* | Roquette (France)* |
| | and/or plasticizer | | |
| Citric acid | Saliva stimulant | N/A* | * |
| Corn starch | Filler or | N/A* | * |
| | Disintegrant | | |
| Microcrystalline | Filler or | PhEur / USP/ | IMCD (UK) |
| cellulose (Avicel | Disintegrant | JPE | (FMC, Dupont) |
| PH-105) | | | |
| Yellow iron oxide | Pigment | USP | Sensient (Germany) |
| | | | (IMCD) |
| Hydrochloric acid | pH adjusting | N/A* | * |
| (HCl) | agent | | |
| Sodium hydroxide | pH adjusting | N/A* | * |
| (NaOH) | agent | | |
| Loparex 78LC1 | Process liner | In-house | Loparex (Netherlands) |
| PET ALU PE | Primary | In-house | AMCOR (Belgium) |
| peelable L188 | packaging | | |
| (laminate) | material | | |
| Purified Water | Process solvent | PhEur | Cooper Industrie (France)* |
| Ethanol | Process solvent | PhEur | Charbonneaux Brabant |
| | | | (France) (Tereos)* |
| 2-propanol | Process solvent | Reagent | VWR (France) |
| | | Laboratory | |
| 1-pentanol | Process solvent | Reagent | Sigma-Aldrich (France) |
| | | Laboratory | |
| Methyl acetate | Process solvent | Reagent | Sigma-Aldrich (France) |
| | | Laboratory | |
| Ethyl acetate | Process solvent | Reag. PhEur, | VWR (France) |
| | | ACS | |
| Isobutyl acetate | Process solvent | Reagent | Sigma-Aldrich (France) |
| | | Laboratory | |
| Acetone | Process solvent | Reag. PhEur/ | VWR (France) |
| | | Reag. USP, | |
| | | ACS | |

*For some materials, the precise quality or supplier are considered to be non-critical for the purpose of conducting the studies in these examples.

### Example 1. Placebo Formulations

It was hypothesized that an oral film containing an API for which a rapid and high systemic in vivo absorption is deemed desirable and biologically possible (considering its physicochemical properties), and for which there may be issues with the patients spitting out, drooling, or swallowing when they shouldn't, should have a moderately high dissolution rate. To achieve such moderately high dissolution rate, it was initially hypothesized that the API should be dissolved in the film, i.e., not suspended as solid particles. Likewise, it was hypothesized that the film should have a modest bioadhesivity, and not be too thick, though yet accommodating the intended dose of the API. Various placebo films were thus prepared, with the aim to identify one or more formulation concepts to progress into the development of active formulations.

### Methods

Film samples were made with the following preparation procedure:
1. The ingredients, except the film-forming polymer(s), were dissolved in water* in a 25 mL glass beaker, during manual mixing, until a homogeneous solution was obtained.
2. The film-forming polymer(s) were added, during manual mixing, until a homogeneous viscous solution was obtained. This solution was denoted “wet mix”. The wet mix batch size was about 12 g. The dry content of the wet mix was between 18-25 wt %.
3. Using a film knife (Adjustable Micrometer Film Applicator, 1117/150 mm, from TQC Sheen, UK) a film of that wet film with thickness of about 500 μm was spread out onto a glass plate. This was made within 48 hours after preparing the wet mix. The resulting film was denoted “wet film”.
4. That wet film was dried during 20-40 minutes at 40-100° C. in a laboratory heating oven (Binder GmbH).
5. The resulting final, dry, film, which had thicknesses between 70-110 μm, was carefully removed from the glass plate, cut into film pieces of about 1.5 cm×2.5 cm. The resulting film, which was denoted “dry film”, or just “film”, was then subjected to tests.

*) In some cases, some ethanol was added to facilitate the subsequent polymer dissolution and swelling

In vitro dissolution rate was not made, as these were placebo without API. However, it was confirmed that all films did readily dissolve in water.

Mechanical properties are assessed with the following two procedures:

Folding endurance: A modified version of a method described by Wasilewska and Winnicka 2019 is used. In this modified method, one film piece is bent at least 10 times back and forth along the length axis, then another piece (from same batch) 10 times back and forth along the width axis, and finally another piece along the diagonal axis. If not breaking, the film is judged to have a good folding endurance; if breaking after three or less bendings, it is judged poor.

Breaking characteristics: One film piece is manually pulled apart in as straight opposite direction as possible. When the film eventually breaks (due to the force and/or due to undeliberate skewing) the breaking line is observed. If breaking according to straight line perpendicular or close thereto, it is judged to have good breaking characteristics. If a very irregular line and/or largely non-perpendicular, it is judged poor.

Overall Mechanical properties were then reported as 1, 2 or 3, where 1 means the desirable outcome of not breaking within 10 bendings and breaking, after pulled apart, along a straight, perpendicular line, and 3 means breaking within 3 bendings and breaking, after pulled apart, along cracked irregular line.

### Results

About 50 different film formulations were prepared, of which a representative selection is presented in the table below. The figures in rows 1-13 refer to the concentration of each component (wt %) in the resulting dry film.

| | P1 | P2 | P3 | P4 | P5 | P6 | P7 | P8 | P9 | P10 | P11 |
|---|---|---|---|---|---|---|---|---|---|---|---|
| Formulation: | | | | | | | | | | | |
| HPMC | | | | | | | 28.3 | | 68.9 | 21.0 | |
| Metolose | | | | | | | | | | | |
| 60SH-50 | | | | | | | | | | | |
| HPMC | | | | | | | 24.4 | | 17.2 | 25.0 | |
| Pharmacoat | | | | | | | | | | | |
| 603 | | | | | | | | | | | |
| HPMC | 92.9 | 100 | 86.0 | 84.0 | 73.0 | 65.8 | | | | | |
| Benecel E-50 | | | | | | | | | | | |
| PVA | | | | | | | | | | | 25.0 |
| Gohsenol EG- | | | | | | | | | | | |
| 05PW | | | | | | | | | | | |
| PVA | | | | | | | | | | | 21.0 |
| Gohsenol EG- | | | | | | | | | | | |
| 40PW | | | | | | | | | | | |
| Pullulan | | | | | | | | 72.7 | | | |
| Glycerol | 5.7 | | 10.0 | 11.0 | 20.0 | 20.2 | 13.9 | 8.1 | 13.9 | 14.0 | 14.0 |
| Citric acid | | | 4.0 | | | | | | | | |
| Polysorbate | 1.4 | | | 5.0 | | | | | | | |
| 80 | | | | | | | | | | | |
| Xylitol | | | | | | 14.0 | | | | | |
| Sorbitol | | | | | 7.0 | | | | | | |
| Corn starch | | | | | | | 16.7 | 9.6 | | 20.0 | 20.0 |
| Microcrystalline | | | | | | | 16.7 | 9.6 | | 20.0 | 20.0 |
| cellulose | | | | | | | | | | | |
| Results: | | | | | | | | | | | |
| Dissolved in | yes | yes | yes | yes | yes | yes | yes | yes | yes | yes | yes |
| water: | | | | | | | | | | | |
| Mechanical | 1 | 2 | 1 | 1 | 1 | 1 | 1 | 2 | 2 | 1 | 1 |
| properties | | | | | | | | | | | |

### Conclusions

It was concluded that:
HPMC, PVA and pullulan appear to be feasible film-forming polymers for the current film formulation, and presumably that other, well-known film-forming polymers, such as for example alginates or polyvinyl alcohol-polyethylene glycol graft copolymers, may also be feasible, and
glycerol is a feasible plasticizer although optimized level was not determined.

While several formulations thus appeared potentially feasible, it was decided to continue studying active formulations based on two concepts: mixtures of the HPMC Metolose 60SH-50 and the HPMC Pharmacoat 603, and mixtures of the PVA Gohsenol EG-05 PW and the PVA Gohsenol EG-40 PW, respectively.

### Example 2. Active Formulations with High Drug Load of Midazolam Hydrochloride

It was realized that in order to achieve a strength of 10 mg midazolam (expressed as the base) and yet having a feasible film thickness allowing for a moderately high film dissolution (e.g., about 90 μm) and a convenient size (e.g., 1.5×2.5 cm), the concentration of midazolam HCl in the dry film must be about 33 wt %. Therefore, active formulations with high drug load (i.e., high API concentration) were made.

### Methods

The preparation procedure as described in Example 1 was used but with the following exceptions: before continuing with step 3, portions of the solutions obtained in step 1 and the wet mix obtained in step 2, respectively, were set aside for being separately studied later. Film dissolution rate or mechanical properties were not assessed. Instead, focus was on microscopic studies of the solution (step 1), the wet mix (step 2) and the film (step 5). Normal light microscopy as well as cross-polarized light microscopy were used.

The figures in rows 1-7 in the table below refer to the concentration of each component (wt %) in the resulting dry film, and rows 8-10 refer to the wet mix.

| | | |
|---|---|---|
| Formulation: | A1 | A2 |

| Midazolam HCl | 34.9 | | 33.1 | |
|---|---|---|---|---|
| HPMC Metolose 60SH-50 | 17.3 | | | |
| HPMC Pharmacoat 603 | 20.5 | | | |
| PVA Gohsenol EG-05PW | | | 28.7 | |
| PVA Gohsenol EG-40PW | | | 23.8 | |
| Glycerol | 11.0 | | 14.4 | |
| Microcrystalline cellulose | 16.2 | | | |
| Wet mix batch size | 12 | g | 25 | g |
| Dry content of wet mix | 28 | wt % | 30 | wt % |
| Midazolam HCl in wet mix | 9.8 | wt % | 9.9 | wt % |

### Results

It was found that the midazolam HCl first dissolved rapidly in the solution in step 1, as expected and previously observed. However, when the solution portion set aside was observed, after 30 minutes, this solution was not clear. Using light microscopy and cross-polarized light microscopy, the occurrence of a precipitate was observed which appeared to be crystalline. Such precipitate was also observed in the wet mix (step 2) and the finished dry films (step 5). It was believed that the precipitate consisted wholly or partly of re-crystallized midazolam although their identity was not further assessed.

### Conclusions

It was concluded that:
despite the high intrinsic dissolution rate and the high apparent solubility of midazolam HCl in water, a clear, stable, particle-free solution is not obtained when dissolving high levels of midazolam HCl in water,
therefore, a significant fraction of the added midazolam HCl may eventually occur as precipitate inside the film which is not desirable, and
solving this problem by significantly lowering the midazolam HCl concentration in the wet mix, for example to 2.5 wt %, is not an option due to the targets and limitations with regard to dry content in wet mix and the eventual drug load in the dry film.

It was decided to study these challenges before continuing with the search for an optimal film formulation.

### Example 3. Solutions and Wet Mixes Containing Midazolam HCl

Solutions and wet mixes were prepared, with the aim to obtain stable, particle-free solutions containing high levels of dissolved midazolam HCl. It was hypothesized that this could be achieved by lowering the pH and/or by using a co-solvent such as for example ethanol.

### Methods

Samples were prepared with the method in Example 1, step 1-2. Visual observation, non-polarized light microscopy and a pH meter were used to assess the results.

### Results

Study of the pH approach: An aqueous solution containing 8.9 wt % midazolam HCl and 4.8 wt % glycerol was prepared. Initially the midazolam appeared to dissolve but a clear stable solution was never obtained. Instead it became a suspension, which had pH3.34. Droplets of 1M HCl was added until a clear stable solution was obtained. That solution had pH1.84. The solution was back-titrated with droplets of 10M NaOH until a precipitation started to occur again, which happened at pH2.76. The study was repeated with a new solution of the same composition, and the results were pH2.94, pH2.00 and pH2.56, respectively.

Study (1) of the ethanol approach: Three wet mixes of about 50 mL were prepared according to the table below. The figures in rows 1-5 refer to wt % in the wet mix, and row 6 describes the solvent composition. No particles were observed in any of WM1, WM2 or WM3.

| Wet mix preparation: | WM1 | WM2 | WM3 |
|---|---|---|---|
| Midazolam HCl | 7.17 | 7.17 | 7.17 |
| HPMC Metolose 60SH-50 | 5.22 | 5.22 | 5.22 |
| HPMC Pharmacoat 603 | 6.30 | 6.30 | 6.30 |
| Glycerol | 3.04 | 3.04 | 3.04 |
| Ethanol:water solvent mix | 78.26 | 78.26 | 78.26 |
| Ethanol in that solvent mix | 80 wt % | 60 wt % | 50 wt % |

Study (2) of the ethanol approach: Three wet mixes of about 50 mL were prepared according to the table below. The figures in rows 1-5 refer to wt % in the wet mix, and row 6 describes the solvent composition. Visual observation and microscopy were used to study the resulting wet mixes after preparation, and also after 3 days. After the observation at Day 3, the wet mixes were put into an oven of 50° C., with no cover, and the solvents were thus allowed to evaporate.

| Wet mix preparation: | WM4 | WM5 | WM6 |
|---|---|---|---|
| Midazolam HCl | 7.10 | 7.10 | 7.10 |
| HPMC Metolose 60SH-50 | 6.66 | 6.66 | 6.66 |
| HPMC Pharmacoat 603 | 6.66 | 6.66 | 6.66 |
| Glycerol | 1.08 | 1.08 | 1.08 |
| Ethanol:water solvent mix | 78.50 | 78.50 | 78.50 |
| Ethanol wt % in that solvent mix | 45 wt % | 40 wt % | 35 wt % |

No particles were observed in any of WM4, WM5 or WM6 after preparation or after 3 days. Upon evaporation in oven at 50° C., a precipitate was however observed in all three vessels, which was believed to consist wholly or partly of midazolam.

### Conclusions

For the pH approach, it was concluded that:
the solution obtained in step 1 of the preparation procedure described in Example 1 preferably should be adjusted to pH2.4 or lower to avoid the occurrence of midazolam-related precipitation, and
the addition of midazolam HCl to water—albeit its pH lowering effect—is not sufficient to create or maintain such pH, i.e., active pH-lowering with HCl or other pH-lowering agent is needed.

For the ethanol approach, it was concluded that:
using ethanol as co-solvent may prevent the occurrence of midazolam-related precipitation,
this can be achieved with an ethanol:water solvent with as little as 35 wt % ethanol, but
at least 45 wt % ethanol is preferable.

### Example 4. HPMC Films with High Drug Load

Based on the findings in Examples 1-3, it was decided to prepare films with HPMC as film-forming polymer, and using both the abovementioned pH approach and the abovementioned ethanol approach.

### Methods

The preparation procedure as described in Example 1 was used but with the following exceptions:
Portions of the solutions obtained in step 1 and step 2, respectively, were set aside for being separately studied later.
For A3: in step 1 of the preparation, the solvent was an ethanol:water solution with 80 wt % ethanol (“the ethanol approach”).
For A4: in step 1 of the preparation, the solvent (water) was first acidified with 1M HCl to achieve pH1.47 (“the pH approach”).

When assessing the results, focus was on general visual appearance, mechanical properties and microscopic studies of the solution set aside from step 1 and step 2. Normal light microscopy as well as cross-polarized light microscopy were used.

### Results

The figures in rows 1-4 in the table below refer to the concentration of each component (wt %) in the resulting dry film, and rows 5-7 refer to the wet mix.

| | A3 | | A4 | |
|---|---|---|---|---|
| Formulation: | (Ethanol approach) | | (pH approach) | |
| Midazolam HCl | 33.3 | | 32.3 | |
| HPMC Metolose 60SH-50 | 28.7 | | 29.9 | |
| HPMC Pharmacoat 603 | 24.0 | | 24.7 | |
| Glycerol | 14.0 | | 13.1 | |
| Wet mix batch size | 25 | g | 14.5 | g |
| Dry content of wet mix | 30.0 | wt % | 25.1 | wt % |
| Midazolam HCl in wet mix | 10.0 | wt % | 8.1 | wt % |

It was found that all wet mixes (step 2) could be made satisfactory according to the preparation procedure. No undissolved particles or precipitation were observed in the solutions set aside from step 1 and step 2.

### Conclusions

It was concluded that:
when using HPMC as the film-forming polymer, both the pH approach and the ethanol approach are feasible.

However, as low pH can sometimes be detrimental to certain excipients as well as to process equipment, it was decided to focus on using the ethanol approach, in combination with HPMC as film-forming polymer, for the next steps in the development of an optimal formulation.

### Example 5. Large Scale Batch Based on HPMC and the Ethanol Approach

Based on the findings in previous Example 4, it was decided to prepare a batch with batch size and equipment typically used for manufacturing oral film batches for the market.

### Methods

The preparation procedure was similar to that described in Example 1 with the exception of size and equipment, and that coating and subsequent drying was a continuous process. Said large scale process is here described:
1. The ingredients, except the film-forming polymer(s), were dissolved in an ethanol:water solvent with 44 wt % ethanol, during mixing, until a homogeneous solution was obtained with no solid content except the pigment. The tank used was equipped with a rotor stator mixer and a scraper blade, and had a volume of 15 L. The order of adding was water, ethanol, glycerol, midazolam HCl and yellow iron oxide, and the mixing speeds varied between 500 rpm to 1300 rpm.
2. The film-forming polymers were added, first Pharmacoat and then Metolose, during mixing, ending with a final thorough mixing using rotor stator speed of 2000 rpm and 20 rpm for the scraper during 52 minutes. The resulting wet mix was left to degas and removing bubbles overnight.
3. Using a continuous coating and drying system, a film batch was manufactured using a unique set of process parameters with regard to tunnel temperature profiles, blade opening and coating speed which determines the time spent in drying tunnel.
4. Said continuous coating and drying system had a length of 12 m. It had four drying areas with individual temperature control system with film unwinding station before tunnel and winding station after tunnel and with a coating station made of a metal cylinder and a blade, which allow to drop a precise quantity of wet mix on the liner.
5. The resulting final, dry, film batch was stored on a mother roll, which was subsequently converted with a slitting and converting equipment in which mother rolls can be split into daughter rolls and the film on a daughter roll can be slit into the desired film piece size (e.g., 1.5×2.5 cm) in which case the process liner can also be removed.
6. After the batch was converted to film pieces, a sufficient number of these film pieces were then packaged into pouches (primary packaging material) using a packaging machine.
7. During step 5 and after step 6, film units were assessed with regard to visual appearance, dry film coating weight (g/m²), loss on drying, residual ethanol (internal gas chromatography method), Assay (midazolam content, by HPLC), in vitro dissolution (Apparatus 2 USP and Eur. Ph. 2.9.3, paddles apparatus including sinker, and UV analysis) and other tests.

A HPLC/UV method was used for the analysis of Assay (midazolam content) and related substances of the oral midazolam films. The samples were first solubilized in solvent such as Acetonitrile/Water-N-Octylamine pH=7.00 (30/70 v/v). The method was developed for concentrations as low as 0.05% of Midazolam (Limit of Quantification LoQ). A weighted linear regression was evaluated over the concentration range 0.2-400.00 g/mL of Midazolam. The chromatographic conditions used by HPLC/UV are summarized below:
Column: HPLC XSelect T3 XP 100 Å, 2.5 μm 2.1×100 mm
Mobile phase A: Water/N-Octylamine pH=7.00 (Buffer)
Mobile phase B: Acetonitrile
Diluent: Acetonitrile/Buffer (30/70 V/V)
Seal washing (vial): Methanol
Flow: 1.4 mL/min
Injection volume: 10 μL
Column temperature: 40° C.
Injector temperature: 15° C.
Wavelength λ/Bandwidth: 240 nm/4 nm
Wavelength Reference/Bandwidth: 360 nm/10 nm
Run time: 35 minutes

### Gradient:

| Time | Mobile | Mobile |
|---|---|---|
| (min) | phase A | phase B |
| 0 | 85 | 15 |
| 2 | 85 | 15 |
| 22 | 70 | 30 |
| 30 | 70 | 30 |
| 30.1 | 85 | 15 |
| 35 | 85 | 15 |

Empower software was used for the calculation of results.

### Results

The figures in rows 1-5 in the table below refer to the concentration of each component (wt %) in the resulting dry film, and rows 6-8 refer to the wet mix.

| Formulation: | A5 | |
|---|---|---|
| Midazolam HCl | 33.0 | |
| HPMC Metolose 60SH-50 | 24.2 | |
| HPMC Pharmacoat 603 | 28.8 | |
| Glycerol | 13.5 | |
| Yellow iron oxide (E172) | 0.5 | |
| Wet mix batch size | 5 | kg |
| Dry content of wet mix | 21.5 | wt % |
| Midazolam HCl in wet mix | 7.1 | wt % |

One wet mix “master batch” was prepared according to materials and methods above for steps 1-2, but the conditions and parameters in steps 3-4 were varied which resulted in eight sub-batches, each one with a unique set of process parameters with regard to tunnel temperature profiles (4 zones, lowest temp 60° C. and highest 110° C.), blade opening (580-620 μm) and coating speed which determines the time spent in drying tunnel (20-40 minutes).

It was found that the most optimal conditions were represented by a sub-batch (here identified as “Trial 7”) that had drying temperatures between 80° C. and 110° C., blade opening of 580 μm and time spent in drying tunnel 40 minutes. The dry film thickness of that sub-batch was manifested as a dry coat weight of 84.6 g/m², and the test for loss-on-drying showed 4.1% and the residual ethanol test showed 3463 ppm. The average weight of one 1.5×2.5 cm film piece was 34.8 mg and the Assay showed an average content of 101.2% of the target value which was 10 mg midazolam (base) per film unit. The in vitro dissolution test showed that 101.4% of target value was released at 10 minutes (which corresponds to about 100% if normalized). Dissolution at 5 minutes was not studied.

In other sub-batches, the test for loss-on-drying showed between 7.0-12.9% and residual ethanol values were between 8 993-31 725 ppm. These sub-batches were not further analysed.

### Conclusions

It was concluded that:
formulation A5 is potentially feasible,
the manufacturing procedure, including the ethanol approach, is feasible, and
process parameters, especially drying conditions, have a large influence over the resulting film quality.

It was decided to continue using this manufacturing procedure including the ethanol approach, and to continue using this formulation concept i.e., HPMC as film-forming polymer, glycerol as plasticizer and a drug load of 33 wt % (as the HCl salt).

### Example 6. Short-Term Physical Stability Study

The aim of this example was to study the short-term physical stability and how it depends on the packaging conditions.

### Methods

A sub-batch, here identified as “Trial 10”, was prepared with the same composition and process as the sub-batch Trial 7 in Example 5, with the exception of blade opening which was 620 μm instead of 580 μm. That formulation is here given the number A6. Film units in step 6 were split into two groups:
Group C-1, which were packaged into sealed pouches intended for made of high barrier laminate manufactured by Danapak Flexibles A/S (Denmark), the same day as manufacturing (day zero).
Group C-3, which was placed in conventional plastic pouches (day zero), and thus not protected from air humidity or oxygen.

At certain timepoints, the film units were taken out of the packages and observed visually with the naked eye as well as with non-polarized light microscopy. The observed film pieces were discarded i.e., not subjected to further observations.

### Results

When observed at day 5, C-1 films showed the desired yellow, smooth appearance with no occurrence of particles, precipitation or other irregularities except for some bubbles which had been formed during manufacturing. The microscopy did not show any particles, precipitation or irregularities either.

C-3 films, on the other hand, showed whitish irregularities, which was also confirmed by microscopy observations of precipitation. It was further believed that the difference between C-1 and C-3 was mainly due to the larger air humidity exposure for the latter.

When observed at day 12, C-1 films showed similar whitish irregularities as did C-3 films at day 5, and similar observations were made by microscopy. For the C-3 films, said structures had continued to develop.

### Conclusions

It was concluded that:
midazolam-related precipitation may be formed upon storage, and
further optimization of the formulation to reduce this phenomenon was needed.

### Example 7. Optimization of Plasticizer Level

It was hypothesized that the type and concentration of plasticizer could have an impact on the undesired formation of midazolam-related precipitation in the dry film, and that the higher the plasticizer concentration, the more formation of precipitate because the plasticizer may increase the mobility of the API molecules within the polymer network and thus facilitating their precipitation. The aim with this example was to test different levels of glycerol as plasticizer.

### Methods

The formulation studied, A7, A8 and A9, had a similar composition as A5 except that the glycerol levels varied. The preparation procedure was similar to that described in Example 1 except for the size and equipment: Mixing equipment was a 4 blade shank, 400 mm diameter with motor from Janke&Kunkel (IKA labortechnik, RW20DZM), the coating equipment was a manual coating table type K control coater (RK Print Coat Instruments Ltd, UK), and the wet mix batch size was 200 g. Assessment methods were visual observation and microscopy.

### Results

The figures in rows 1-5 in the table below refer to the concentration of each component (wt %) in the resulting dry film, and rows 6-8 refer to the wet mix.

| Formulation: | A7 | | A8 | | A9 | |
|---|---|---|---|---|---|---|
| Midazolam HCl | 33.0 | | 33.0 | | 33.0 | |
| HPMC Metolose 60SH-50 | 26.5 | | 31.0 | | 33.5 | |
| HPMC Pharmacoat 603 | 26.5 | | 31.0 | | 33.5 | |
| Glycerol | 14.0 | | 5.0 | | 0.0 | |
| Yellow iron oxide (E172) | 0.0 | | 0.0 | | 0.0 | |
| Wet mix batch size | 200 | g | 200 | g | 200 | g |
| Dry content of wet mix | 21.5 | wt % | 21.5 | wt % | 21.5 | wt % |
| Midazolam HCl in wet mix | 7.1 | wt % | 7.1 | wt % | 7.1 | wt % |

The mechanical properties were assessed with the method described in Example 1. It was found that both A7 and A8 had good mechanical properties, ranked as 1 according to Example 1. Formulation A9, on the other hand, ranked as 2 and especially it did not break well along a straight line. When cut with razor blade, the cut edge had an uneven, cracky character, which was not the case for A7 and A8.

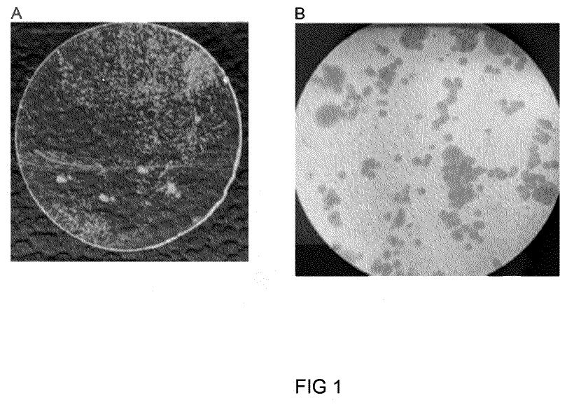

After one week of storage at 25° C. in a conventional plastic pouch (i.e., not protected against air and humidity exposure), A7, but not A8 or A9, had developed the whitish character observed also in Example 6. This is shown in FIG. 1. Similar observations were made after two weeks.

From these results it can be seen that 5 wt % glycerol is preferred over with 0 wt % and 14 wt % because 5 wt % combines acceptable mechanical properties (i.e., plasticizing effect) with an acceptable stability.

### Conclusions

It was concluded that:
formulation A8 (5 wt % glycerol) is superior to formulations A7 (14 wt %) and A9 (0 wt %), as well as to the formulation A5 (13.5 wt %) that was made in large industrial scale in Example 5.

It was decided to use the same composition as formulation A8, for the next, large scale, batch.

### Example 8. Large Scale Batch Based on HPMC and the Ethanol Approach and 5 wt % Plasticizer

Example 5 had confirmed the feasibility of the manufacturing process and Example 7 had identified a new and more feasible composition. These two results were now combined.

### Methods

The formulation studied, A10, had a similar composition as A8 in Example 7, with the exception that 1 wt % pigment was added. The preparation procedure was similar to that described in Example 5, with the exception that blade opening for wet film coating as 620 μm and that only one film batch was made from the wet mix batch (i.e., not several sub-batches). The same assessment methods as in Example 5 and 7 were used, including the Assay. A stability study was also started.

The dissolution rate was measured for formulation A10, using the USP Dissolution Apparatus 2—(Paddles apparatus) (37° C.±0.5° C.). The dissolution testing was performed at 75 rpm in 1000 mL phosphate buffer pH 6.8 with Tween 20 at 0.08%. The content of midazolam was determined by using UV spectroscopy (240 nm).

### Results

The figures in rows 1-5 in the table below refer to the concentration of each component (wt %) in the resulting dry film, and rows 6-9 refer to the wet mix.

| Formulation: | A10 | |
|---|---|---|
| Midazolam HCl | 33.0 | |
| HPMC Metolose 60SH-50 | 30.5 | |
| HPMC Pharmacoat 603 | 30.5 | |
| Glycerol | 5.0 | |
| Yellow iron oxide (E172) | 1.0 | |
| Ethanol in the ethanol:water solvent | 45 | wt % |
| Wet mix batch size | 5 | kg |
| Dry content of wet mix | 21.5 | wt % |
| Midazolam HCl in wet mix | 7.1 | wt % |

The solution obtained in step 1 (i.e., step 1 of the preparation procedure described in Example 5) before adding the pigment was checked for the absence of precipitation by visual observation and microscopy and so was the wet mix obtained in step 2. The final, stable pH of the solution as well as the wet mix was pH3.3.

The dry film thickness of the batch was manifested as a dry coating weight of 89.1 g/m² (target was 90.0 g/m²), and the test for loss-on-drying showed 3.9% and the residual ethanol test showed 14 365 ppm. The Assay showed an average content of 99.8% of the target value which was 10 mg midazolam (base) per film unit. The dissolution results are summarized in the table below and were considered to show a moderately high dissolution rate.

Amount dissolved (%)

| | | | | |
|---|---|---|---|---|
| Time | Minimum | Maximum | Mean | Normalized |

| | | | | | |
|---|---|---|---|---|---|
| 5 | min | 82.6 | 90.6 | 87 | 88.8 |
| 10 | min | 91.5 | 95.8 | 94 | 95.9 |
| 15 | min | 94.4 | 97.1 | 96 | 98.0 |
| 30 | min | 95.8 | 98.1 | 97 | 99.0 |
| 45 | min | 96.2 | 98.4 | 97 | 99.0 |
| 60 | min | 96.3 | 98.6 | 97 | 99.0 |
| 120 | min | 96.5 | 98.9 | 98 | 100.0 |
| 240 | min | 96.8 | 99.3 | 98 | 100.0 |
| 360 | min | 97.0 | 99.6 | 98 | 100.0 |

### Stability Study:

After 12 months storage at 5° C. and ambient % RH, the Assay was 101.2%, Total related substances were 0.06%, loss-on-drying was 4.7%, and the visual appearance was compliant (criteria: “yellow-orange to slightly brown, opaque rectangular film”). Dissolution after 5 minutes was 90% (Mean), and the dissolution rate remained being moderately high.

After 6 months storage at 40° C./75% RH, the Assay was 97.8%. However, the visual appearance was not fully compliant: there was a development of whitish spots such as those described in Example 6 and 7, which again was interpreted as some kind of precipitation involving midazolam and being a sign of insufficient physical stability.

### Conclusions

It was concluded that:
the composition and the preparation method described in this Example 8 are feasible for a film containing dissolved midazolam hydrochloride and having a moderately high dissolution rate,
the resulting product would be feasible for being studied in human clinical studies, but that
the development of precipitation at higher storage temperatures needs to be further studied.

### Example 9. Testing Triethyl Citrate (TEC) as Plasticizer

There was an aim to study potential improvements in the long-term physical stability. In Example 7 it was shown that the concentration of plasticizer had an impact on the long-term stability, and therefore it was hypothesised that also the type of plasticizer could matter. TEC has been proposed in the literature as an effective plasticizer for oral films.

### Methods

A preparation method as in Example 7 was used. The formulations studied, A11-A13, had a similar composition as in Examples 7 and 8, except that the type and level of plasticizer were different. Assessment methods were mechanical properties, visual observation and microscopy, as previously described. Focus was on the occurrence of undesired precipitation and its potential growth over time. There were thus no attempts to make pharmaceutical analyses such as assay or in vitro dissolution because it was not believed that the precise type and level of plasticizer would have any major impact on those properties.

### Results

The figures in rows 1-5 in the table below refer to the concentration of each component (wt %) in the resulting dry film, and rows 6-9 refer to the wet mix.

| Formulation: | A11 | | A12 | | A13 | |
|---|---|---|---|---|---|---|
| Midazolam HCl | 33.0 | | 33.0 | | 33.0 | |
| HPMC Metolose 60SH-50 | 28.5 | | 31.0 | | 31.5 | |
| HPMC Pharmacoat 603 | 28.5 | | 31.0 | | 31.5 | |
| Triethyl citrate (TEC) | 10.0 | | 5.0 | | 3.0 | |
| Yellow iron oxide (E172) | 0.0 | | 0.0 | | 1.0 | |
| Wet mix batch size | 60 | g | 60 | g | 200 | g |
| Ethanol in the ethanol:water | 50 | wt % | 50 | wt % | 45 | wt % |
| solvent | | | | | | |
| Dry content of wet mix | 21.5 | wt % | 21.5 | wt % | 21.5 | wt % |
| Midazolam HCl in wet mix | 7.1 | wt % | 7.1 | wt % | 7.1 | wt % |

Firstly, it was found that both A11 and A12 had good mechanical properties, ranked as 1 according to the principles laid out in Example 1. There were some minor observations that Formulation A12 had cracky edges upon cutting, but A12 was nevertheless deemed satisfactory with regard to the mechanical properties. Next, the short-term stability of A11 and A12, as measured with visual observation and microscopy, was studied after storage at 5° C., 25° C./60% RH and 40° C./75% RH for up to 16 weeks. At 25° C./60% RH there was a significant difference between A11 (10 wt % TEC) and A12 (5 wt %), with A11 showing whitish appearance and precipitation observed by microscopy, i.e., clear indications of inferior stability.

As A12 (5 wt % TEC) thus appeared viable both with regard to mechanical properties and physical stability, it was realized that an even lower level of TEC could prove to be even better.

Therefore, formulation A13 with 3 wt % TEC was prepared next. The resulting film was however quite brittle, and during the folding test described in Example 1, several samples broke after just one bending. It was therefore realized that 3 wt % TEC was not a sufficient level for achieving acceptable mechanical properties.

### Conclusions:

It was concluded that:
using 10 wt % TEC as plasticizer is too high and using 3 wt % is too low, if the aim is to achieve a viable product with comparable characteristics as A10.

### Example 10. Testing Other Potential Plasticizers

Examples 7 and 9 demonstrated that the type and level of plasticizer are critical attributes of a midazolam film, and that above a certain level of plasticizer there is a risk for the development of midazolam-related precipitation. The aim of this example was to study other potential plasticizers than glycerol and TEC.

### Methods

Preparation methods as in Example 9 was used. Assessment methods were, as applicable, as in Example 9: mechanical properties, visual observation and microscopy, as previously described. As in Example 9, focus was on the occurrence of precipitation over time.

### Results

The figures in rows 1-6 in the table below refer to the concentration of each component (wt %) in the resulting dry film, and rows 7-10 refer to the wet mix.

| Formulation: | A14 | | A15 | | A16 | |
|---|---|---|---|---|---|---|
| Midazolam HCl | 33.0 | | 33.0 | | 33.0 | |
| HPMC Metolose 60SH-50 | 28.5 | | 31.0 | | 31.0 | |
| HPMC Pharmacoat 603 | 28.5 | | 31.0 | | 31.0 | |
| Sorbitol | 10.0 | | 0.0 | | 0.0 | |
| Poloxamer 407 | 0.0 | | 5.0 | | 0.0 | |
| Kollicoat IR | 0.0 | | 0.0 | | 5.0 | |
| Wet mix batch size | 60 | g | 200 | g | 200 | g |
| Ethanol in the ethanol:water | 50 | wt % | 45 | wt % | 45 | wt % |
| solvent | | | | | | |
| Dry content of wet mix | 21.5 | wt % | 21.5 | wt % | 21.5 | wt % |
| Midazolam HCl in wet mix | 7.1 | wt % | 7.1 | wt % | 7.1 | wt % |

It was found that A14 (sorbitol, 10 wt %) resulted in very brittle films, which were so poor that they were not even subjected to further mechanical testing. A formulation

A 14b with 5 wt % sorbitol was also made but showed similar poor mechanical properties.

It was found that A15 (poloxamer 407, 5 wt %) resulted in films with very good mechanical properties, which, when subjected to mechanical testing according to Example 1, could be folded more than 10 times without breaking and which broke in a straight line when pulled apart. However, upon 4 weeks storage at 25° C./60% RH, the films became whitish and the characteristic, undesired precipitation were observed inside the film when using microscopy.

It was found that A16 (Kollicoat IR, 5 wt %) resulted in films with similar good mechanical properties as A15. Already after the preparation, the visual appearance was somewhat whitish but in a homogenous way that was not believed to indicate midazolam-related precipitation, and no such precipitation could be seen with microscopy. Upon 4 weeks storage at 25° C./60% RH, the films still had that satisfactory visual appearance and precipitates or structures were still not observed in microscopy. However, after 24 weeks, precipitates inside the films were observed when using microscopy, and this precipitation—contrary to the initial whitish appearance of the films—was believed to be midazolam-related. The mechanical properties remained good.

### Conclusions

It was concluded that:
sorbitol is not an effective plasticizer in a film containing high level of midazolam and using HPMC as film-forming polymer,
poloxamer 407 and Kollicoat IR are effective plasticizers at levels as low as 5 wt %, in a film containing high level of midazolam and using HPMC as film-forming polymer, and
Kollicoat IR (5 wt %) results in a somewhat better stability than poloxamer 407 (5 wt %) but yet not fully satisfactory.

### Example 11. Kollicoat IR as Film-Forming Polymer

Kollicoat IR has been proposed as a film-forming polymer for oral films, which is something different from Kollicoat IR being used as an added plasticizer alongside another film-forming polymer, as was the case for formulation A16 in Example 10.

### Methods

Preparation method as in Example 10 was used, except for ethanol level and dry content as explained below.

### Results

The figures in rows 1-2 in the table below refer to the concentration of each component (wt %) in the resulting dry film, and rows 3-6 refer to the wet mix.

| Formulation: | A17 | |
|---|---|---|
| Midazolam HCl | 33.0 | |
| Kollicoat IR | 67.0 | |
| Wet mix batch size | 200 | g |
| Ethanol in the ethanol:water solvent | 34 | wt % |
| Dry content of wet mix | 27.3 | wt % |
| Midazolam HCl in wet mix | 9.0 | wt % |

Compared with Examples 8, 9 and 10, the ethanol level was lower and the dry content of wet mix was higher. These levels had been determined in placebo experiments preceding this example but not being presented here. The aim was to render the wet mix a viscosity that was feasible for coating into a wet film; Kollicoat IR is different than the previously used HPMC mix in that respect.

It was found that A17 resulted in a film with very good mechanical properties, which were similar to those reported for A15 and A16 in Example 10. However, already soon after the preparation, i.e., without even a short-term storage test, there were whitish spots in the film that were interpreted as midazolam-related precipitation.

### Conclusions

It was concluded that:
Kollicoat IR as the sole film-forming polymer is not feasible, for a film containing dissolved midazolam hydrochloride.

### Example 12. Film with Suspended Midazolam Base

As it had been found challenging to formulate a film containing dissolved midazolam hydrochloride and at the same time having a satisfactory long-term physical stability, it was hypothesized that the midazolam should instead be in the suspended state inside the film, i.e., as the same solid particles that was added as API raw material. However, midazolam hydrochloride was not believed to be feasible for that purpose because it is highly soluble in water and would dissolve in the water during the film preparation and thus not ending up as the desired solid suspended particles inside the film.

However, it was decided to study the feasibility of midazolam for that purpose. The solubility of midazolam base in water is very low and it would presumably not dissolve during the film preparation.

### Methods

The preparation procedure was similar to that in Example 1, except that no solution portions were set aside and that the wet mix batch size was about 100 g. Formulations with and without glycerol was made, as glycerol could potentially be a co-solvent for midazolam base in water.

X-ray powder diffraction (XRPD) was also used to study the physical stability. The XRPD instrument was a PanAlytical X'Pert Pro. XRPD measurements were performed using a Cu-anode (45 kV/40 mA), a Kα-1 Johansson monochromator (1.540598 Å) and a Pixcel detector. The 2-theta range was 2-35° using a scan speed of 0.03°/s and a step size of 0.013°. Three repetitions were performed for each sample, generating a total scan time of 1 hour and 1 minute. Slow spinning sample holders were used. The samples were attached as pieces of film on the zero background wafers of Si which had been, using double adhesive tape. The measurements were performed using a programmable incident divergency slit.

The compositions are described in the table below.

| | A18 | | A19 | |
|---|---|---|---|---|
| | wt % in | wt % in | wt % in | wt % in |
| Formulation: | wet mix | dry film | wet mix | dry film |
| Midazolam base | 7.1 | 33.0 | 7.1 | 33.0 |
| HPMC Metolose 60SH-50 | 7.2 | 33.5 | 6.1 | 28.5 |
| HPMC Pharmacoat 603 | 7.2 | 33.5 | 6.1 | 28.5 |
| Glycerol | 0.0 | 0.0 | 2.2 | 10.0 |
| Water | 78.5 | N/A | 78.5 | N/A |
| Dry content of wet mix | 21.5 | N/A | 21.5 | N/A |

### Results

The visual appearance of the wet mix was whitish, which was interpreted as a suspension and thus being in line with the intentions of this experiment. Microscopy observation of the wet mix as well as of the finished film confirmed a dominating occurrence of API crystals. The finished dry films were whitish and non-transparent and with a somewhat granular surface. All these observations thus indicated that a film with suspended API had been prepared, as intended. There was no significant effect of the glycerol, except that the finished films of formulation A19 was less brittle as could be expected from the addition of a plasticizer. The films were put on a stability. After 24 weeks of storage at room temperature and at 40° C./75% RH, there were no changes of the films as could be assessed by visual appearance or microscopy.

However, it was realized that such visual observations are not sufficient for studying the fate of the API. For example, hypothetically it could happen that the API partly dissolves during the preparation and then precipitates into a different polymorph than the one added as API ingredient. Therefore, XRPD was used to study samples of formulation A19 according to the following procedure:

1) An XRPD diffractogram for the API raw material (midazolam base) had been recorded beforehand.

2) An initial diffractogram of the film sample was recorded, with the sample being taken fresh out of the water- and air-tight pouch.

3) It was noted that these two diffractograms corresponded very well, i.e., it was confirmed that nothing had happened to the API during manufacturing.

4) The sample was stored at Ambient temperature and 65% relative humidity (Amb/65% RH) for 4 weeks. During storage awaiting next XRPD timepoint, the sample was mounted on a holder and was not in a pouch or otherwise protected against that humidity.

5) A new diffractogram was recorded. It was noted that this new diffractogram corresponded very well with the diffractograms from step 1) and 2).

6) A new 4 weeks period at the same storage conditions, followed by yet another diffractogram which again showed that nothing had happened.

7) Storage conditions were now stepped up to 40° C./75% RH, and a new 4 weeks period followed.

8) Again, the diffractogram did not reveal any changes of the API inside the film.

9) Finally, a 2 weeks period at the very challenging condition of 40° C./89% RH, but the final diffractogram again showed that nothing had happened.

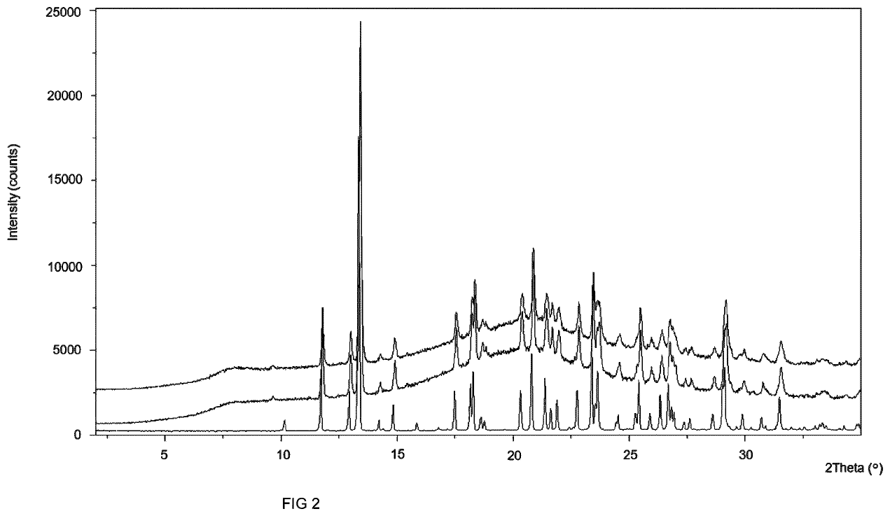

The diffractograms from steps 1), 2) and 9) are shown in FIG. 2.

### Conclusions

It was concluded that:
it is possible to use the solvent casting method to prepare a film with midazolam base as API and water as solvent, if the intention is that the API should be suspended inside the film and not dissolved, and
such film appears to have excellent physical stability.

### Example 13. Film with Suspended Midazolam Base—In Vitro Dissolution Studies

As the aqueous solubility of midazolam base is quite low, the in vitro dissolution rate of the film prepared in Example 12 also had to be studied. Because, the overall aim was to develop a film with moderately high dissolution rate, as opposed to a very slow dissolution rate that may sometimes occur if the API has a low aqueous solubility and/or a low intrinsic dissolution rate.

### Methods

The in vitro dissolution Apparatus 2 USP and Eur. Ph. 2.9.3 (paddles apparatus), with sinkers, was used. Agitation speed was 75±3 rpm, the dissolution medium volume was 1000 mL, the temperature was 37.0° C.±0.5° C., and sampling times were 5, 10, 15, 30 and 45 minutes and then 1, 2, 4 and 6 hours. Two different dissolution media were used: Water, and phosphate buffer pH 6.8+Tween 20 at 0.08%, respectively. To compensate for minor content differences between the film samples, all results were normalized to a terminal release level of 100%.

### Results

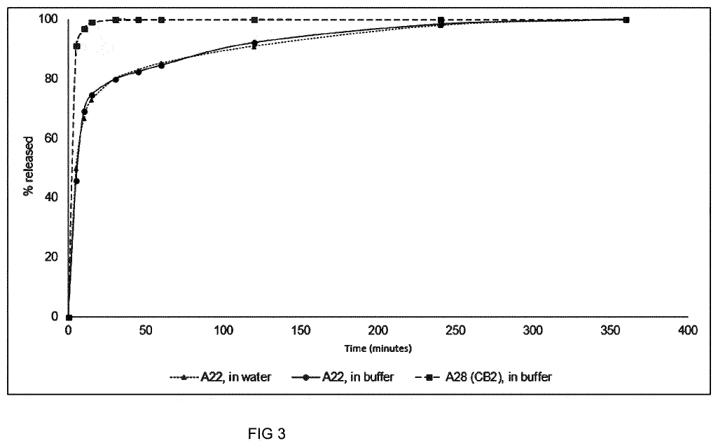

The results for formulation A18 are shown in FIG. 3, in which also a dissolution curve for the clinical batch used in Example 23 is shown, i.e., formulation A24 (also called CB2, as in “clinical batch 2”). It can be seen that formulation A18 does not meet the herein defined criteria for a/moderately high dissolution rate. After 5 minutes less than 50% had been released and 100% release was no achieved until at least 3 hours. This would rather, with the terminology used in the present application, be categorized as a very slow dissolution. For A24, on the other hand, about 91% had been released at 5 minutes, time to 100% was between 15-30 minutes, and it did meet the herein defined criteria for a moderately high dissolution rate.

### Conclusions

It was concluded that:
a film with suspended midazolam base as API is not feasible if the intention is to have a moderately high dissolution rate.

### Example 14. Screening for Feasible Process Solvents

It was hypothesized that with a nonaqueous solvent that does not dissolve midazolam hydrochloride, it would be possible to prepare films in which the midazolam hydrochloride ingredient would be predominately present inside the finished film in the suspended state and also keeping the same polymorphic form as the API ingredient used. Other requirements on such solvent would be that it is volatile, and non-toxic.

### Methods

For each solvent, four test tubes were prepared, containing about 1 g solvent and midazolam hydrochloride at amounts of about 0.1 wt %, 1 wt %, 4 wt % and 7 wt %, respectively. The tubes were thoroughly mixed and were then visually observed and photographed. After 15 minutes of resting, they were again visually observed and photographed, and were then mixed again. After another 1 hour, they were finally visually observed and photographed. It was noted whether there were any undissolved solid material remaining, approximately how much, and whether it had sedimented at the bottom or formed a whitish suspension, and if that suspension was translucent or opaque. No analytical method, e.g., HPLC Assay, was used to actually quantify the precise solubilities because the main aim was just to determine whether the midazolam hydrochloride was predominately dissolved or solid, at 4 wt % and 7 wt % respectively, which represent typical levels for the midazolam hydrochloride content in a wet mix. If midazolam hydrochloride was predominately solid at 7 wt %, the solvent was deemed to be potentially very feasible.

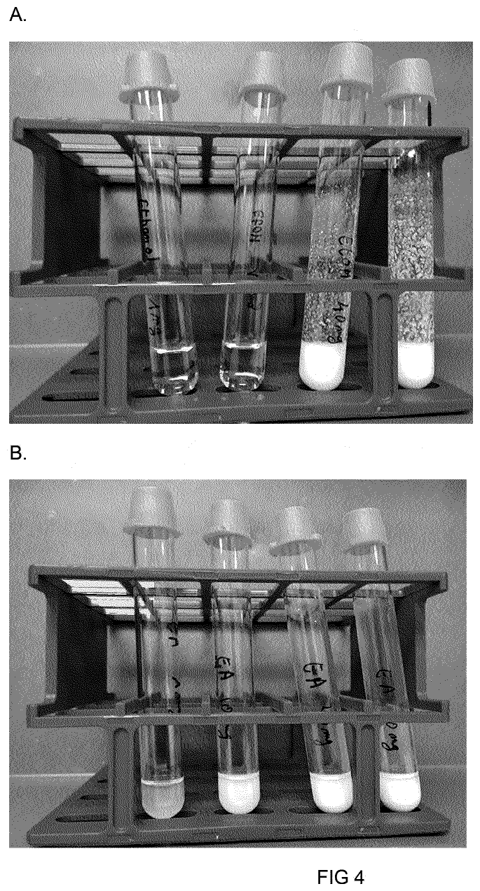

The experimental setup is illustrated by FIG. 4, which shows the appearance at the last observation, for test tubes with ethanol and ethyl acetate, respectively.

### Results

Solvents studied were ethanol, 2-propanol, 1-pentanol, methyl acetate, ethyl acetate, isobutyl acetate and acetone.

Ethanol was included despite the fact that it had previously been found to be a co-solvent which, together with water, improves the solubility of midazolam hydrochloride. The reason for yet screening it was that pure ethanol may potentially have different solubilizing capacity than an ethanol-water mix.

The results and the interpretations are presented in the table below.

| | Visual appearance at last observation | | | | |
|---|---|---|---|---|---|
| Solvent | 0.1 wt % | 1 wt % | 4 wt % | 7 wt % | Tentative interpretation |
| Ethanol | clear | clear | opaque | opaque | Solubility is between 1-4 wt %. |
| | | | | | Solvent is not feasible. |
| 2-propanol | clear | translucent | opaque | opaque | Solubility is between 0.1-1 |
| | | | | | wt %. Solvent is potentially |
| | | | | | feasible. |
| 1-pentanol | clear | translucent | opaque | opaque | Solubility is between 0.1-1 |
| | | | | | wt %. Solvent is potentially |
| | | | | | feasible. |
| Methyl | cloudy | opaque | opaque | opaque | Solubility is ≤0.1 wt %. Solvent |
| acetate | | | | | is potentially very feasible. |
| Ethyl | cloudy | opaque | opaque | opaque | Solubility is ≤0.1 wt %. Solvent |
| acetate | | | | | is potentially very feasible. |
| Isobutyl | cloudy | opaque | opaque | opaque | Solubility is ≤0.1 wt %. Solvent |
| acetate | | | | | is potentially very feasible. |

| | | |
|---|---|---|
| Acetone | clear supernatant over sedimented | Solubility is ≤0.1 wt %. Solvent |
| | material, and amount of sedimented | is potentially very feasible. |
| | material was proportional to the wt % | |
| | of API | |

### Conclusions

Methyl acetate, ethyl acetate, isobutyl acetate and acetone were deemed to be potentially very feasible, for being process solvents in solvent casting preparations of oral films in which the midazolam hydrochloride should remain solid and suspended inside the film. 2-propanol, 1-pentanol were also deemed to be potentially feasible.

### Example 15. XRPD of Non-Dissolved Material from the Solvent Screening

It was realized that the terminal observation of on-dissolved material in the experiments presented in Example 14 is not unanimous proof that the added API material has not dissolved and/or undergone changes. For example, it could happen that the added API material initially dissolves, partly or wholly, and then precipitates into a new solid material which is then erratically recorded as undissolved API. The assessment method in Example 14 can thus be supplemented with XRPD studies.

### Methods

Test tubes prepared with ethanol and ethyl acetate, respectively, corresponding to the last step in Example 14, were prepared and allowed to sediment overnight. The solvents in each sample of sedimented material were slowly evaporated off at ambient temperature and atmospheric pressure. It was then subjected to the XRPD method described in Example 12 except that only steps 1-2 of that method were performed, and that the reference “API raw material” recorded in step 1 was midazolam hydrochloride and not midazolam base.

### Results

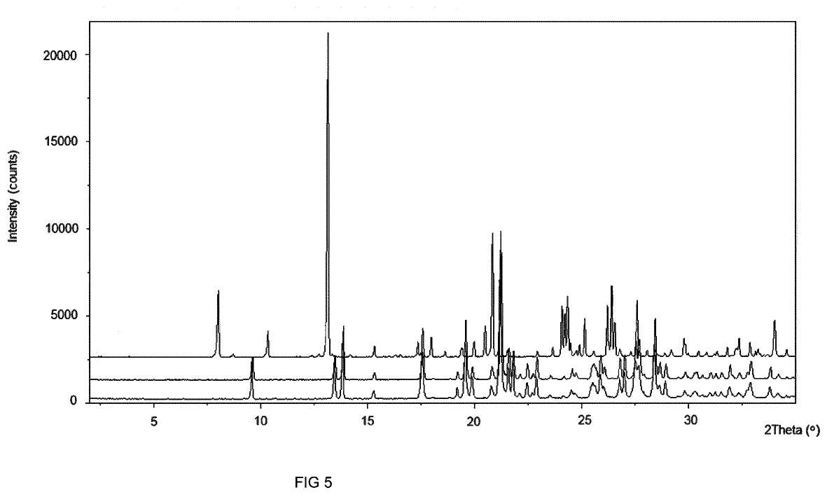

The results are presented in FIG. 5. It can be seen that for ethanol, the sediment consisted predominately of a solid crystalline material that was not midazolam hydrochloride. For ethyl acetate, however, the sedimented material appeared to be identical to midazolam hydrochloride.

### Conclusions

It was concluded that:
ethyl acetate was a potentially very feasible solvent, and
the occurrence of undissolved material in a solvent screening with midazolam hydrochloride is not an unanimous proof that the added midazolam hydrochloride has not been dissolved.

### Example 16. Devising a Preparation Method with Ethyl Acetate as Process Solvent

After thus identifying a number of solvents with potential feasibility, an attempt was made to use one of these solvents, ethyl acetate, for preparing a film with the solvent casting method which should contain suspended midazolam hydrochloride that has not undergone changes compared with the added API ingredient. As the first step in devising such preparation method, the appropriate concentration of the HPMC mix in the wet mix had to be determined, because with a new solvent, the optimal viscosity for film casting may be achieved by some other polymer concentration than the typical 14 wt % used in Examples 5, 8, 9 and 12.

### Methods and Results

As a start, a 50:50 mix of HPMC Metolose 60SH-50 and HPMC Pharmacoat 603 was prepared, and added at an amount of about 14 wt % to ethyl acetate. Metolose 60SH-50 and HPMC Pharmacoat 603 has the HPMC substitution pattern 2910, and has viscosities of 50 mPas and 3 mPaS, respectively.

It was surprisingly found that ethyl acetate was not able to dissolve that 50:50 mix. Dissolving either HPMC Metolose 60SH-50 or HPMC Pharmacoat 603 was not possible either.

Ethanol was then included as co-solvent, at levels of 5 wt % and 24 wt %, respectively (despite the risk that it might also dissolve the API which in this case was not desirable). The 50:50 mix of HPMC Metolose 60SH-50 and HPMC Pharmacoat 603 still did not dissolve.

Water was then included as co-solvent, at levels of 9, 14 and 19 wt %, respectively (despite the risk that it might also dissolve the API). The 50:50 mix of HPMC Metolose 60SH-50 and HPMC Pharmacoat 603 still did not dissolve.

### Conclusions

It was concluded that:
HPMC Metolose 60SH-50 and/or HPMC Pharmacoat 603 appeared not to be feasible as film-forming polymers in solvent casting preparation of oral films when ethyl acetate is the process solvent.

### Example 17. Screening for Feasible, Non-Cellulosic Film-Forming Polymers

Due to the surprising failure to use HPMC Metolose 60SH-50 and/or HPMC Pharmacoat 603 in the previous Example 16, a number of non-cellulosic polymers were screened for their ability to be dissolved in ethyl acetate.

### Methods

22 mL test tubes were prepared containing about 9.5 g ethyl acetate and about 6 wt % of the polymers. The solution was then thoroughly mixed, then sonicated, standing overnight, and sonicated again, all of which took place at room temperature. The occurrence and amount of non-dissolved material was then assessed.

### Results

The following polymers were not soluble at a level about 6 wt % in ethyl acetate.
Acrylates: Eudragit L 100-55; Kollicoat IR; Pemulen TR-1 (a carbomer)
Alginates: Sodium alginate
Others, of natural origin: Kappa-Carragenan; lota-Carragenan; Pullulan; Rheocare XGN (a xantan gum)
PVA: PVA EG-05-; PVA EG-40 PW
Others, of synthetic origin: PVP 360

Four acrylate polymers were however shown to be soluble:
Methacrylic acid-methyl acrylate copolymers: Eudragit E 100; Eudragit RS 100; Eudragit RL 100; Eudragit RL PO.
Polyvinyl caprolactam-polyvinyl acetate-polyethylene glycol graft copolymer. Soluplus

Eudragit E 100 and Eudragit RS 100 appeared most soluble and were thus tested also at a concentration of about 33 wt %. They proved to be soluble also at that level. Clear solutions were formed, and the resulting viscosity appeared to be feasible for film making. Films were made ad hoc from those viscous solutions and became clear albeit somewhat brittle.

For Soluplus, the experimental setup was different: about 1 g ethyl acetate was used, and Soluplus was added at amounts of about 1 wt %, 5 wt %, and 20 wt %, respectively. Soluplus was soluble at least up to 20 wt % in ethyl acetate.

### Conclusions

It was concluded that:
not all non-cellulosic film-forming polymers are soluble in ethyl acetate, but
Soluplus, Eudragit E 100, Eudragit RS 100, Eudragit RL 100 and Eudragit RL PO are soluble in ethyl acetate and are potentially feasible for the subsequent film casting.

### Example 18. Screening for Feasible, Cellulosic Film-Forming Polymers

As evident from many examples above, most of the previous development work had been made with HPMC as film-forming polymer and hence it would be advantageous to use a HPMC or other cellulosic polymer, despite the fact that HPMC Metolose 60SH-50 and HPMC Pharmacoat 603 were not feasible.

### Methods

Between 7-14 wt % of the polymers were added to ethyl acetate of a volume of about 50 mL, which was then thoroughly mixed, and finally observed visually by the naked eye.

### Results

The hypromellose acetate succinate AQOAT AS-LG dissolved at a level of 17 wt %, to a [placebo] wet mix with feasible viscosity
The hypromellose Affinisol HPMC HME 100LV dissolved but already at the 7 wt % level the viscosity was very high
The hypromellose Affinisol HPMC HME 15LV dissolved at a level of 10 wt %, to a [placebo] wet mix with feasible viscosity
Mixes of Affinisol HPMC HME 15LV: AQOAT AS-LG at ratios of 1:9, 3:7, 5:5 and 7:3: they all dissolved at levels between 10-17 wt %, to [placebo] wet mixes with variable but feasible viscosities, though also a bit granular
The hydroxyethylcellulose Natrosol 250L did not dissolve at the level of 14 wt %
The hydroxyethylcellulose Natrosol 250HX did not dissolve at the level of 14 wt %

Films were made ad hoc from the viscous solutions of Affinisol HPMC HME 15LV, AQOAT AS-LG and those of the Affinisol HPMC HME 15LV: AQOAT AS-LG mixes that had ratios of 1:9, 3:7, 5:5 and 7:3. Acceptable film with good mechanical properties were obtained from all those mixes. However, the films containing AQOAT AS-LG appeared to have a very slow dissolution rate in in water, meaning that they may not be ideal for preparing oral films, although that problem could potentially be overcome by the use of other excipients (e.g., disintegration or solubilization agents) or be less of a problem in buffered solutions or in vivo where the dissolution environment is saliva.

### Conclusions

It was concluded that:
not all cellulosic film-forming polymers are soluble in ethyl acetate,
Affinisol HPMC HME 100LV and Affinisol HPMC HME 15LV are soluble in ethyl acetate and are potentially feasible for subsequent solvent casting of films, and
AQOAT AS-LG is soluble in ethyl acetate and may be feasible, for example, with the help of other excipients.

### Example 19. Preparing Films Using Affinisol HPMC HME 15LV and Ethyl Acetate

It had been concluded that a film with suspended midazolam hydrochloride inside can be prepared if using a process solvent that does not dissolve the midazolam hydrochloride but does dissolve the film-forming polymer used. One such combination of solvent and polymer was now tested: ethyl acetate and Affinisol HPMC HME 15LV. Ethanol as minor co-solvent to ethyl acetate was also tested, as it was known from previous studies with water as solvent that ethanol can have a beneficial effect on the dissolution of HPMC.

### Methods

The same preparation procedure and assessment methods as in Example 1 was used, except that the wet mix batch size was about 150 g. Samples of the solution from step 1 (i.e., before the polymer was added) was set aside for being studied by microscopy, and samples of the wet mix obtained in step 2 was set aside for being studied with visual observation and non-polarized light microscopy.

### Results

Three wet mixes were prepared according to the table below, but only one (A22) progressed to preparation of a film.

| | A20 | | A21 | | A22 | |
|---|---|---|---|---|---|---|
| | wt % in | wt % in | wt % in | wt % in | wt % in | wt % in |
| Formulation: | wet mix | dry film | wet mix | dry film | wet mix | dry film |
| Midazolam | 4.0 | N/A | 4.0 | N/A | 4.0 | 33.0 |
| hydrochloride | | | | | | |
| Affinisol | 8.0 | N/A | 8.0 | N/A | 8.0 | 67.0 |
| HPMC HME | | | | | | |
| 15LV | | | | | | |
| Ethyl acetate | 87.1 | N/A | 83.6 | N/A | 88.0 | 0.0 |
| Ethanol | 0.9 | N/A | 4.4 | N/A | 0.0 | 0.0 |
| Dry content of | 12.0 | N/A | 12.0 | N/A | 12.0 | N/A |
| wet mix | | | | | | |

When studied by microscopy, it was seen that the solutions obtained in step 1 contained particles of the API midazolam hydrochloride, as expected. However, the amount of such particles was less in A21 than in A20, and less in A20 than in A22, i.e., adding ethanol was not feasible because it seemed to dissolve the API which in this context is not desirable.

The appearance in microscopy for the solution obtained in step 1 for A22 was compared with the appearance in microscopy for the API raw material used, with the same magnification. The appearances were the same, i.e., there were no signs that the API had firstly dissolved (partly or wholly) and then precipitated, which in this context would not have been desirable.

For A22, the preparation thus continued to step 2 and the visual appearance of the wet mix was whitish, opaque and homogeneous, i.e., it did appear to be a suspension, as intended.

The wet mix of A22 then continued to step 3-5. The resulting film was whitish and opaque, and had good mechanical properties, e.g., it could be bent back and forth several times without breaking, and when teared, it broke along a rather straight line. When studied in microscopy, particles could be observed which had the same shape and size as those of the API raw material used and those in the solution from step 1, i.e., there were still no signs that the API had firstly dissolved (partly or wholly) and then precipitated.

### Conclusions

Satisfactory films could be made using the solvent casting method and a process solvent that does not dissolve the midazolam hydrochloride but does dissolve the film-forming polymer used.
One such combination is: ethyl acetate as solvent and Affinisol HPMC HME as film-forming polymer.

### Example 20. XRPD Studies of Film Obtained in Example 19

The results from Example 19 indicated that formulation A22 was satisfactory and that the API had not undergone any changes during the manufacturing. To further support that it had not undergone any changes during the manufacturing, XRPD was applied to A22.

### Methods

Formulation A22 was studied. The same XRPD method and equipment as in Example 12 were used.

### Results

1) An XRPD diffractogram for the API raw material (midazolam hydrochloride) had been recorded beforehand.

2) An initial diffractogram of the film sample was recorded, with the sample being taken fresh out of the water- and air-tight pouch.

3) It was noted that these two diffractograms corresponded very well, i.e., it was confirmed that nothing had happened to the API during manufacturing.

4) The sample was stored at Ambient temperature and 65% relative humidity (Amb/65% RH) for 4 weeks. During storage awaiting next XRPD timepoint, the sample was mounted on a holder and was not in a pouch or otherwise protected against that humidity.

5) A new diffractogram was recorded. It was noted that this new diffractogram corresponded very well with the diffractograms from step 1) and 2).

6) A new 4 weeks period at the same storage conditions, followed by yet another diffractogram which again showed that nothing had happened.

7) Storage conditions were now stepped up to 40° C./75% RH, and a new 4 weeks period followed.

8) Again, the diffractogram did not reveal any changes of the API inside the film.

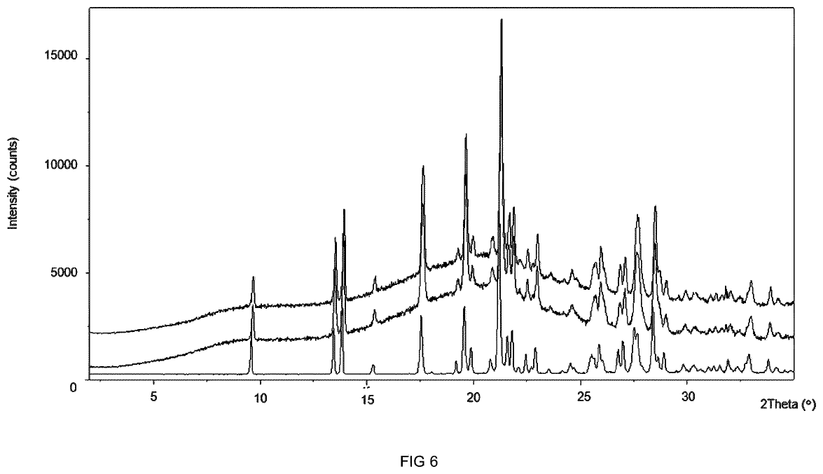

The diffractograms from steps 1), 2) and 8) are shown in FIG. 6.

### Conclusions

It was concluded that:
in formulation A22, prepared as described in Example 19, the suspended API has not undergone any changes during preparation of the film and appears stable also during storage.

### Example 21. In Vitro Dissolution Studies of Film Obtained in Example 19

Example 13 had shown that even if an oral film, that contains suspended API, apparently has satisfactory characteristics, appears stable, and can be readily manufactured, it may yet not have the desired dissolution rate which in this case is a moderately high dissolution rate. Therefore, formulation A22 from example 19 was now subjected to in vitro dissolution studies.

### Methods

Apparatus 2 USP and Eur. Ph. 2.9.3 (paddles apparatus), with sinkers, was used. Agitation speed was 75±3 rpm, the dissolution medium volume was 1000 mL, the temperature was 37.0° C.±0.5° C., and sampling times were 5, 10, 15, 30 and 45 minutes and then 1, 2, 4 and 6 hours. Two different dissolution media were used:

Water, and phosphate buffer pH 6.8+Tween 20 at 0.08%, respectively. To compensate for minor content differences between the film samples, all results were normalized to a terminal release level of 100%.

### Results

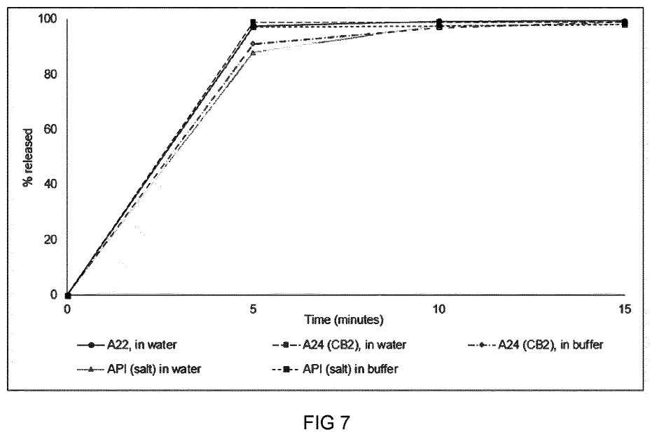

FIG. 7 shows the results for the formulation A22 which was prepared in Example 19, being compared with formulation A24 from Example 23, as well as with the non-formulated API midazolam hydrochloride. The five curves included in the figure almost coincide and no major differences in the dissolution rates can be concluded. Formulation A24 is the clinical batch (CB2) used in the human comparative bioavailability study in Example 23, and has the same composition, size and manufacturing process as the clinical batch (CB1) used in the human comparative bioavailability study in Example 22.

The precise dissolution results for A22 were 97.5% at 5 minutes and 99.2% at 10 minutes, and it thus met the herein defined criterium for moderately high dissolution rate.

### Conclusions

The dissolution of the API midazolam hydrochloride does not seem to depend on whether the dissolution medium is buffer or water,
formulation A22 has the same in vitro dissolution characteristics as formulation A24,
formulation A22 is a satisfactory formulation with a satisfactory preparation method, and
if and when tested on human subjects, formulation A22 is likely to produce similar beneficial clinical results as those shown in Example 22 and 23.

### Example 22. Human Bioavailability Study with Buccal Administration

In Example 8, it was shown that an oral film containing 10 mg midazolam (added as midazolam hydrochloride) and having moderately high dissolution rate could be successfully manufactured in large scale (formulation A10). In Example 19, an alternative way to prepare such film was successfully tested (formulation A22). Example 20 indicated that the physical stability of that film (A22) was satisfactory, and in Example 21 it was confirmed that its dissolution rate was moderately fast.

The aim in this Example 22 was to study the bioavailability of an oral film with 10 mg midazolam (added as midazolam hydrochloride) that has a moderately high dissolution rate. The bioavailability was studied after buccal administration and was compared with an oromucosal solution which was also administered buccally at a dose of 10 mg midazolam.

### Methods

A batch (formulation A23, also called CB1) with the same size, composition, materials and manufacturing process as batch A10 in Example 8 was made, with the exception that A23 was made under GMP conditions and was intended for clinical trials.

After being analysed similarly to Example 8 and subject to other quality and GMP related controls and procedures, the batch was approved and released for being used in human clinical trials. A comparative bioavailability study was then made at well-renowned clinical contract research organization (CRO) located in the Czech Republic. The study was a non-blinded, single dose, randomized, three treatment, three period, cross-over study. Twentyfour (24) healthy, adult, male volunteers were included in the study after being assessed with regard to a number of inclusion and exclusion criteria. These inclusion and exclusion criteria were related the general health status as well as aspects related specifically to the treatments (e.g., hypersensitivity to midazolam). The treatments were given when the study subjects were in fasting state, i.e., had not eaten for several hours before the dosing. The study complied with ICH E6 (R2) Guideline for Good Clinical Practice. The Declaration of Helsinki, as last amended and accepted by the 64th World Medical Association General Assembly, Fortaleza, Brazil, October 2013, as well as other applicable guidelines, directives and regulations. Two of the three treatments were:
T1: The oral midazolam film, with 10 mg midazolam (base), applied on the buccal mucosa of the inside of one cheek (“unilateral administration”).
R: Buccolam oromucosal solution, of the 10 mg midazolam (base) strength which has a volume of 2 mL, half of which was applied on the buccal mucosa of the inside of one cheek and the other half in the other cheek (“bilateral administration”) in accordance with the accompanying user instructions.

For both treatments T1 and R, the subjects were instructed not to intentionally swallow neither the product nor the saliva solution that is being accumulated due to the natural, continuous saliva production, because such swallowing would decrease the overall bioavailability since the oral-gastrointestinal bioavailability of midazolam is lower than the buccal-transmucosal bioavailability. However, the subjects were instructed to empty their mouth of excess saliva at 5 minutes and at 10 minutes after the products had been administered. That instruction was made mainly for ethical reasons and study compliance reasons; excess saliva has to go somewhere, if not swallowed. However, that instruction also mimicked drooling (i.e., saliva running out from the mouth), which often occurs in patients with seizures, although that was not the main intention with the instruction.

The instructions given about not swallowing and about mouth emptying were the same for both products.

After being given the treatment, blood samples were withdrawn at 0.16, 0.33, 0.50, 0.67, 0.83, 1.00, 1.25, 1.50, 1.75, 2.00, 2.50, 3.00, 4.00, 6.00 and 8.00 hours post-dose. Including the pre-dose sample (within 1.00 h before dosing), the total number of blood collections in each study period was thus 16.

A HPLC/MS/MS method was used for the bioanalysis of these samples. The samples were first isolated from plasma by protein precipitation. The analytical methods then used 50 μL of plasma sample for each analysis. The method was validated for concentrations as low as 0.20 ng/ml of midazolam in plasma. A weighted linear regression was evaluated over the concentration range 0.20-200.00 ng/ml of midazolam in plasma. The equipment used for HPLC/MS/MS method (here identified as HPLC/MS/MS TSQ-08) was:
HPLC system: pumps ACCELA 1250 and ACCELA 600 (Flux Instruments), autosampler PAL HTS-xt (CTC Analytics) and ten-port switching valve SelectPro (Alltech)
MS detector: TSQ Vantage (ThermoFisher Scientific)
Guard column: Luna C18 (2) Mercury, 20×4.0 mm, 5 μm, Phenomenex
Column: Kinetex PhenylHexyl, 100×3 mm, 5 μm, Phenomenex
Injection: 10 μL
Acquisition: mode: HESI; scan: MS/MS (SRM)
Mobile phase: ACN, MeOH, 160 mM HCOONH4, water

In addition to the experimental samples corresponding to the abovementioned timepoints, each subject also provided samples for suitability test, plasma blank, zero sample, calibration samples, and quality control samples.

Phoenix WinNonlin software was used for the pharmacokinetic parameters calculation based on the bioanalytical results. A non-compartmental model for evaluation in plasma after single-dose extravascular dosing using Linear Trapezoidal/Linear Interpolation calculation method was used. The best-fit method with uniform weighting and without any exclusion was used for the terminal elimination rate constant calculation in all cases. The drug concentration in plasma at each sampling time point was presented for each product for each subject. The descriptive statistics: arithmetic mean, standard deviation, coefficient of variation, maximal value, minimal value, median value, and geometric mean were also presented. Data were summarized as the concentration versus time profiles for each product in graphs for each Subject as well as for mean values.

### Results

### Analysis of the Batch:

The analytical results were: Assay 97.4%, Total related substances were 0.16%, Dissolution after 10 minutes was 96% (dissolution at 5 minutes was not measured), loss-on-drying was 3%, residual ethanol was <30 000 ppm, Total aerobic microbial count (TAMC) was <1 cfu/g, Total combined yeasts and moulds count (TYMC) was <1 cfu/g, total absence of Staphylococcus aureus and Pseudomonas aeruginosa, and the visual appearance was compliant.

### Results of the Comparative Bioavailability Study:

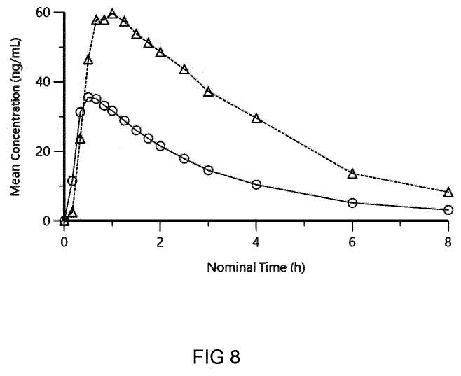

All the included 24 subjects carried through the whole study and were subject to the eventual statistical evaluation. The results are presented in FIG. 8 as the graph of mean values. The geometric least squares mean for Cₘₐₓ (ng/ml) and AUC₀₋ₜ were:

| | T1 | R |
|---|---|---|
| | (film, 10 mg | (oromucosal solution, |
| Treatment: | midazolam) | 10 mg midazolam) |
| Cₘₐₓ (ng/mL) | 64.32 | 35.58 |
| AUC₀₋ₜ (ng*h/mL) | 223.65 | 102.71 |

### Conclusions

It was concluded that:
the oral film had a significantly higher bioavailability than the buccal solution, after both products being buccally administered at a dose of 10 mg and being subject to the same instructions to the study subjects, and that
a possible explanation for this result is that the fraction of the total given dose that is lost due to the prescribed mouth emptying, is lower for the film than for the oromucosal solution, and that
these results supported the hypothesis the oral midazolam film should have a moderately high dissolution rate.

### Example 23. Human Bioavailability Study with Oral Administration

In Example 22 it was shown that the bioavailability of the oral film after buccal administration was higher than that of an oromucosal solution of the same dose after buccal administration. That result was interpreted as a support for the hypothesis that the oral film should have a moderately high dissolution rate, because if it would have had instantaneous in vivo dissolution, the fraction of the total given dose that is lost due to the prescribed mouth emptying would have been the same as for the oromucosal solution.

The aim in this Example 23 was to study the bioavailability after oral administration of an oral film with 10 mg midazolam (added as midazolam hydrochloride) that has a moderately high dissolution rate and compare it with an oral solution which was also administered orally at a dose of 10 mg midazolam.

Contrary to oromucosal solutions, oral solutions are meant to be swallowed and not staying in the mouth.

### Methods

A batch (A24, also called Clinical Batch 2, CB2) with the same size, composition, materials and manufacturing process as batch A23 (CB1) in Example 22 was manufactured, under GMP conditions.

After being analysed similarly to Example 22 and subject to other quality and GMP related controls and procedures, the batch was approved and released for being used in human clinical trials. A comparative bioavailability study was then made at well-renowned clinical contract research organization (CRO) located in the Czech Republic. The study was a non-blinded, single dose, randomized, four treatments, four period, cross-over study. Twelve (12) healthy, adult, male volunteers were included in the study after being assessed with regard to a number of inclusion and exclusion criteria. These inclusion and exclusion criteria were related the general health status as well as aspects related specifically to the treatments (e.g., hypersensitivity to midazolam). The treatments were given when the study subjects were in fasting state, i.e., had not eaten for several hours before the dosing. The study complied with ICH E6 (R2) Guideline for Good Clinical Practice. The Declaration of Helsinki, as last amended and accepted by the 64th World Medical Association General Assembly, Fortaleza, Brazil, October 2013, as well as other applicable guidelines, directives and regulations. Two of the four treatments were:
T2: The oral midazolam film, with 10 mg midazolam (base), applied onto the tongue.
R1: Midazolam Hydrochloride Syrup, with 2 mg/mL midazolam (base), manufactured by Hikma Pharmaceuticals USA Inc and purchased on the US market. It was administered as 5 mL i.e., 10 mg midazolam, applied by a plastic syringe into the mouth according to the product information.

(The other treatments were 5 mg doses of the film and an intramuscular injection, respectively, but these are not further described or discussed here).

For both treatment T2 and R1, the subjects were instructed not to spit out any product or saliva or otherwise empty their mouths from excess saliva during the study, which is the normal practice for clinical trials of oral products.

After being given the treatment, blood samples were withdrawn at 0.16, 0.33, 0.50, 0.67, 0.83, 1.00, 1.25, 1.50, 1.75, 2.00, 2.50, 3.00, 4.00, 6.00 and 8.00 hours post-dose. Including the pre-dose sample (within 1.00 h before dosing), the total number of blood collections in each study period was thus 16.

A HPLC/MS/MS method was used for the bioanalysis of these samples, which was the same as the one used in Example 22. The same software and procedures as in Example 22 were used for the statistical assessment and the pharmacokinetic parameters.

### Results

### Analysis of the Batch:

The analytical results were: Assay 101.2%, Total related substances were 0.07%, Dissolution after 10 minutes was 97% (dissolution at 5 minutes was not measured), loss-on-drying was 3%, residual ethanol was <30 000 ppm, Total aerobic microbial count (TAMC) was <1 cfu/g, Total combined yeasts and moulds count (TYMC) was <1 cfu/g, total absence of Staphylococcus aureus and Pseudomonas aeruginosa, and the visual appearance was compliant.

### Results of the Comparative Bioavailability Study:

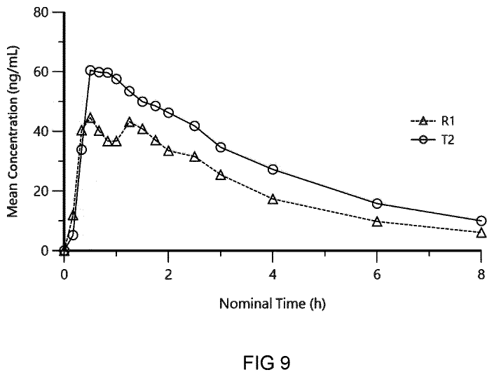

10 subjects carried through the whole study and were subject to the eventual statistical evaluation. The results are presented in FIG. 9 as the graph of mean values. The geometric least squares mean for Cₘₐₓ (ng/ml) and AUC₀₋ₜ were:

| | T2 | R1 |
|---|---|---|
| | (film, 10 mg | (oral solution, |
| Treatment: | midazolam) | 10 mg midazolam) |
| Cₘₐₓ (ng/mL) | 68.72 | 49.36 |
| AUC₀₋ₜ (ng*h/mL) | 218.97 | 144.44 |

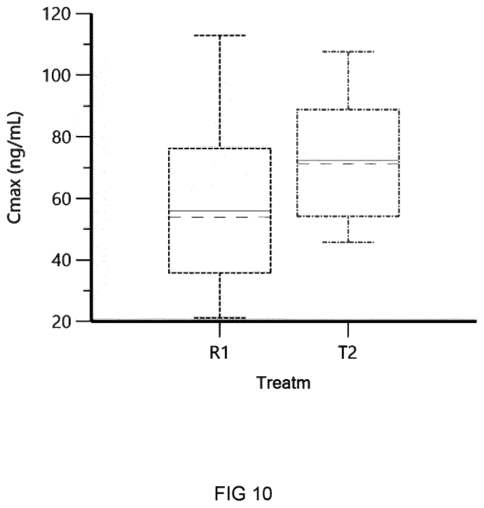

It was also noted that the oral film (T2) produced a lower variability in the data compared with the oral solution (R1). This is evident from FIG. 10, including the fact that the highest individual Cₘₐₓ values was higher for the oral solution than for the film, despite the lower mean and median Cₘₐₓ for the oral solution. High variability and high outliers for Cₘₐₓ are typically not beneficial for safety reasons, neither for midazolam nor for most other products, and therefore these results were clearly in favour for the oral film.

It can also be noted, in FIG. 9, that the oral solution (R1) produced a double-peak phenomenon, which was not the case for the oral film (T2). These two plasma peaks for R1 occurred at 0.5 hours and 1.25 hours, respectively, and have comparable peak heights. This phenomenon may occur after oral administration of midazolam and other benzodiazepines and is believed to be a result of reduced gastric motility caused by muscle relaxant effect of benzodiazepines (Belle et al. 2002). It is typically not a beneficial effect, because when oral midazolam is administered to give moderate sedation, it is preferable not just to have a predictable time to onset of action but also to have a predictable duration of action. A second plasma peak, which may or may not occur and which may have variable magnitude, does not contribute to that predictability. Therefore, oral administration of the oral midazolam offers yet another advantage over oral administration of the oral solution.

### Conclusions

It was concluded that:
the oral film had a significantly higher bioavailability than the oral solution, after both products being orally administered at a dose of 10 mg,
a possible explanation for the film's higher bioavailability is that the fraction of the total given dose that is “lost” due to the lower bioavailability in the gastrointestinal tract as compared with the oral cavity, is lower for the film than for the oral solution,
these results further supported the hypothesis that the oral midazolam film should have a moderately high dissolution rate, and that
the oral film offers a way to avoid the disadvantageous double-peak phenomena observed for other orally administered midazolam products.

### Example 24. Studying the HPC Solubility in Ethyl Acetate

It was hypothesized that, based on its molecular properties, hydroxypropyl cellulose (HPC) could potentially be a feasible film-forming polymer for the present invention. It was thus subject to screening procedure proposed by the present inventors. Firstly, it was screened for its ability to be dissolved in ethyl acetate.

### Methods

The material used was hydroxypropyl cellulose (HPC) of the brand name Klucel EF and Klucel ELF, manufactured and provided by Ashland Industries Europe (Schaffhausen, Switzerland).

Samples of HPC Klucel EF and Klucel ELF were prepared in 100 ml glass bottles with screw caps. Ethyl acetate was added during gentle stirring until a homogeneous opaque viscous gel was obtained with a viscosity that was considered appropriate for making films. For Klucel EF that appropriate concentration for making films was found to be about 15% (w/w) and for Klucel ELF about 20% (w/w). However, these levels were determined ad hoc and was not optimized.

A slight separation of ethyl acetate (i.e. supernatant) was noted for both samples after about 1 week at room temperature.

### Results

It was found that HPC Klucel ELF was soluble in ethyl acetate at least up to 20 wt % and HPC Klucel EF was soluble in ethyl acetate at least up to 15 wt %.

### Conclusions

It was concluded that hydroxypropyl cellulose is sufficiently soluble in ethyl acetate and can thus be a feasible film-forming polymer for the present invention.

### Example 25. Preparing Films Based on HPC

Films containing midazolam hydrochloride and HPC Klucel ELF as the film-forming polymer were then prepared.

### Methods

Film samples were made with the following preparation procedure:

1) 1.00 g of midazolam was weighed with 2.38 g of HPC Klucel ELF in a 15 mL glass jar with a plastic screw cap and carefully mixed with a spatula. The homogeneous dry mixture was then suspended in 8.53 g of ethyl acetate during manual mixing.

2) A homogeneous viscous suspension was obtained in which it appeared as if the HPC was dissolved and the midazolam was solid i.e. in the same state as it was added. This solution was denoted “wet mix”. The wet mix batch size was about 11.9 g. The dry content of the wet mix was about 28.4 wt %.

3) Using a film knife (Adjustable Micrometer Film Applicator, 1117/150 mm, from TQC Sheen, UK) a film of that wet film with a thickness that became of about 600-900 micrometer was spread out onto a glass plate. This was made within 24 hours after preparing the wet mix. The resulting film was denoted “wet film”.

4) That wet film was dried during 40 minutes at 40° C. in a laboratory heating oven (Binder GmbH).

5) The resulting final, dry, film, which had an average coating weight of about 177 g/m², was carefully removed from the glass plate, cut into film pieces of about 1.5 cm×2.5 cm. The resulting film was denoted “dry film”, or just “film”.

6) It was estimated that the concentration of midazolam hydrochloride in the dry film was about 29 wt %.

### Results

A film was obtained with acceptable mechanical properties. It had a whitish appearance which indicated that the midazolam hydrochloride was predominately in the suspended state i.e. not dissolved.

### Conclusions

It was concluded that hydroxypropyl cellulose is a feasible film-forming polymer for the present invention.

### Example 26. XRPD Studies of Film Obtained in Example 26

The results from Example 25 indicated that said formulation was satisfactory. And furthermore, it indicated the desired outcome that the API had not undergone any unwanted changes during the manufacturing. To further support that the API had not undergone any changes during the manufacturing, XRPD was applied to a sample from Example 25.

### Methods

The same XRPD method and equipment as in Example 12 were used.

### Results

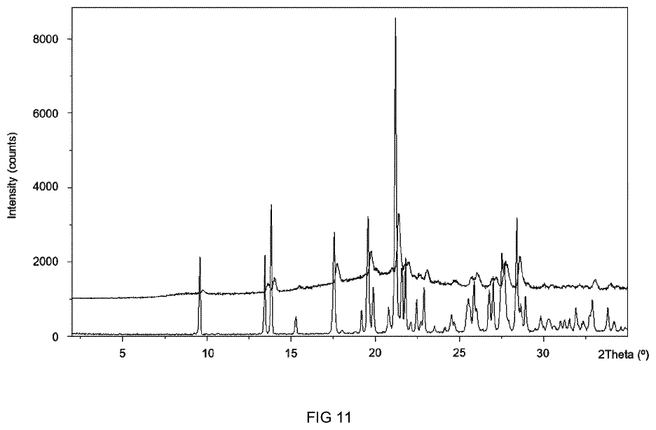

1) An XRPD diffractogram for the API raw material (midazolam hydrochloride) had been recorded beforehand, which is the bottom diffractogram in FIG. 11 and which is the same (except the scale) as in FIG. 6.

2) An initial diffractogram of the current film sample was recorded, with the sample being taken fresh out of the water- and air-tight pouch in which it had been stored at ambient temperatures for about 2 weeks since manufacturing. See the top diffractogram in FIG. 11.

3) It was noted that these two diffractograms corresponded very well, i.e., it was confirmed that nothing had happened to the API during manufacturing (e.g. recrystallization into another crystal form).

4) It was noted that there was a coherent shift in the precise positions, for each corresponding peak, between the bottom and top diffractograms. Likewise, a difference in the peak widths and heights. This is however explained by the fact that the film sample of the top diffractogram varied in thickness and that it was not perfectly mounted on the XRPD sample holder. It was thus not an indication that the two diffractograms did not correspond.

### Conclusions

It was concluded that in the film formulated with HPC as film-forming polymer in Example 25, the suspended midazolam hydrochloride has not undergone any changes during preparation of the film. This further supported the approach to use HPC as film-forming polymer for the present invention.

### REFERENCES

Jithendra et al., Panacea Journal of Pharmacy and Pharmaceutical Sciences (2015), 4:4, 801-816
Soroushnai et al., Current Drug Delivery (2018), 15, 9, 1294-1304
Wasilevska, K. et al., Acta Pharm. (2019), 69, 155-176
WO 2017/009446
Rogawski et al., Epilepsy & Behavior (2019), Volume 101, Part B, 106537
CN 1 830 447 A
U.S. Pat. No. 10,744,086 B2
U.S. Pat. No. 11,173,114 B1
US 2017/0119660 A1
U.S. Pat. No. 11,173,114 B1
Kathpalia H. and Gupte A, Current Drug Delivery (2013), 10, 667-684
Belle, D. J. et al., J Clin Pharmacol (2002), 53, 67-74

1. A unit dosage form in the form of an oral film comprising:
a) at least 20 wt % (defined as the base) midazolam or a pharmaceutically acceptable salt thereof; and
b) 35 to 80 wt % of a film-forming polymer which is soluble in ethyl acetate.

2. The unit dosage form according to claim 1, wherein the film-forming polymer is selected from the group consisting of:
i. HPMC 2528;
ii. HPC;
iii. methacrylic acid-methyl acrylate copolymers; and
iv. polyvinyl caprolactam-polyvinyl acetate-polyethylene glycol graft copolymer.

3. The unit dosage form according to claim 1, wherein the film-forming polymer is HPMC 2528.

4. The unit dosage form according to claim 1, wherein the film-forming polymer is HPC.

5. The unit dosage form according to any one of claims 1 to 4, wherein the pharmaceutically acceptable salt is midazolam hydrochloride or midazolam maleate.

6. The unit dosage form according to any one of the preceding claims, wherein the unit dosage form comprises 2.5 to 20 mg (defined as the base) of midazolam or a pharmaceutically acceptable salt thereof, such as 5 to 15 mg, such about 10 mg of midazolam or a pharmaceutically acceptable salt thereof.

7. The unit dosage form according to any one of the preceding claims, wherein the unit dosage form comprises or consists of 30 to 40 wt % midazolam or a pharmaceutically acceptable salt thereof and 60 to 80 wt % film-forming polymer, such as about 33 wt % midazolam or a pharmaceutically acceptable salt thereof and 60 to 80 wt % film-forming polymer.

8. The unit dosage form according to any one of the preceding claims, wherein at least 80 wt %, such as at least 90 wt %, such as at least 95 wt %, such as at least 98 wt % of the total amount of midazolam or a pharmaceutically acceptable salt thereof is suspended in the film of the unit dosage form.

9. The unit dosage form according to any one of claims 1 to 8, wherein at least 85% of the midazolam, or pharmaceutically acceptable salt thereof, has been dissolved within 10 minutes but no more than 97.5% has been dissolved within 5 minutes in the USP Dissolution Apparatus 2—Paddle, 37° C.±0.5° C., 75 rpm in 1000 mL water.

10. A process for producing a unit dosage form in the form of an oral film according to any one of claims 1 to 9, wherein the process comprises the sequential steps of:
a) mixing midazolam or a pharmaceutically acceptable salt thereof and one or more film-forming polymers in one or more process solvent(s) to provide a wet mix wherein the film-forming polymer(s) are dissolved but the midazolam or pharmaceutically acceptable salt thereof is not dissolved;
b) casting the wet mix obtained in step a) to provide a wet film;
c) drying said wet film to obtain a dry film; and
d) cutting the dry film of step c) into a unit dosage form.

11. The process according claim 10, wherein step a) comprises the sequential steps of:
i. mixing the film-forming polymer in the process solvent to obtain a homogenous solution;
ii. adding the midazolam or pharmaceutically acceptable salt thereof and optionally one or more plasticizers, colorants and/or flavoring agents to the solution in i) and mixing to obtain a wet mix in which the film-forming polymer is dissolved but midazolam or pharmaceutically acceptable salt thereof is not dissolved.

12. The process according claim 10, wherein step a) comprises the steps of:
i. mixing the midazolam or pharmaceutically acceptable salt thereof with the process solvent to obtain a homogenous suspension;
ii. optionally adding one or more plasticizers, colorants and/or flavoring agents to the suspension of i) and mixing to obtain a homogenous mixture; and
iii. adding the film-forming polymer to the mixture of ii) and mixing to obtain a wet mix in which the film-forming polymer is dissolved but midazolam or pharmaceutically acceptable salt thereof is not dissolved.

13. The process according to any one of claims 10 to 12, wherein the process solvent comprises or consists of one or more non-aqueous solvents selected from the group consisting of ethyl acetate, acetone, 1-butanol, 2-butanol, butyl acetate, isobutyl acetate, isopropyl acetate, methyl acetate, methyl ethyl ketone, 2-methyl-1-propanol, 1-pentanol, 1-propanol, 2-propanol, propyl acetate and trimethylamine.

14. The process according to any one of claims 10 to 13, wherein the process solvent is ethyl acetate.

15. The process according to any one of claims 10 to 14, wherein the pharmaceutically acceptable salt of midazolam is midazolam hydrochloride.

16. The process according to any one of claims 10 to 15, wherein the film-forming polymer is selected from the group consisting of:
i. HPMC 2528;
ii. HPC;
iii. methacrylic acid-methyl acrylate copolymers; and
iv. polyvinyl caprolactam-polyvinyl acetate-polyethylene glycol graft copolymer.

17. The process according to any one of claims 10 to 15, wherein the film-forming polymer is HPMC 2528.

18. The process according to any one of claims 10 to 15, wherein the film-forming polymer is HPC.

19. The unit dosage form according to any one of claims 1 to 9 for use in the treatment of seizures and/or for induction of moderate sedation or pre-sedation in a subject.
